(19) United States
(12) Patent Application Publication
(10) Pub. No.: US 2019/0177284 A1
(43) Pub. Date: Jun. 13, 2019

(54) TETRAZOLYL-CONTAINING CYCLOPROPANECARBOXAMIDES

(71) Applicant: Bayer Pharma Aktiengesellschaft, Berlin (DE)

(72) Inventors: Stefan Bäurle, Berlin (DE); Jens Nagel, Daxweiler (DE); Andrea Rotgeri, Berlin (DE); Adam James Davenport, Abingdon (GB); Christopher Charles Stimson, Abingdon (GB)

(21) Appl. No.: 16/211,572

(22) Filed: Dec. 6, 2018

(30) Foreign Application Priority Data
Dec. 13, 2017 (EP) 17207113.6

(51) Int. Cl. C07D 257/04; C07D 405/12; C07D 401/12

(52) U.S. Cl. CPC C07D 257/04; C07D 401/12; C07D 405/12

(57) ABSTRACT

The present invention relates to tetrazolyl-containing cyclopropanecarboxamides compounds of general formula (I) as described and defined herein, to pharmaceutical compositions and combinations comprising said compounds and to the use of said compounds for manufacturing a pharmaceutical composition for the treatment or prophylaxis of a disease, in particular of neurogenic disorder, as a sole agent or in combination with other active ingredients.

The present invention relates to tetrazolyl-containing cyclopropanecarboxamides of general formula (I) as described and defined herein, to pharmacological compositions and combinations comprising said compounds and to the use of said compounds for manufacturing a pharmaceutical composition for the treatment or prophylaxis of a disease or disorder and for the treatment of pains, which are associated with such diseases, in particular of Bradykinin B1 receptor associated disorders which are related to inflammation or at least partially driven by neurogenic events like diseases related to chronic pain or frequent pain conditions like but not restricted to osteoarthritis, rheumatoid arthritis, gout, inflammatory bowel disease, and endometriosis and diseases related to Bradykinin B1 receptor activation and/or up-regulation in affected tissue like but not restricted to asthma, fibrosis in various tissues or diabetes as a sole agent or in combination with other active ingredients.

### BACKGROUND OF THE INVENTION

The present invention relates to chemical compounds that antagonize the effects of human Bradykinin B1 receptor (Gene Name BDKRB1, Gene ID 623).

The Bradykinin B1 receptor is a membrane-bound G-protein coupled receptor, which is linked to a second messenger system that triggers increase of intracellular calcium concentrations. The main signalling pathway is linked to Gq protein and phospholipase C (Leeb-Lundberg, L. M. et al. (2005), Pharmacol Rev 57(1): 27-77). Activation of Bradykinin B1 receptor has been shown to be pro-algesic, pro-fibrotic, and proinflammatory while Bradykinin B1 receptor antagonists had clear anti-inflammatory and analgesic effects in various animal models (Gougat, J. B. et al. (2004), J Pharmacol Exp Ther 309(2): 661-669; Dias, J. P. et al. (2007), Br J Pharmacol 152(2): 280-287; Schuelert, N. et al. (2015), Eur J Pain 19(1): 132-142). As consequence of Bradykinin B1 receptor activity increased gene expression and protein levels of proinflammatory cytokines like e.g. 11-6 and 11-8 that attract and activate inflammatory leucocytes, increase of PGE2 (Prostaglandin 2) levels and therefore activation of the inflammation related prostaglandin pathway, phosphorylation and upregulation of TRPV1 (Transient Receptor Potential Vanilloid 1) receptors which are important mediators of pain transduction and induction of neurogenic inflammation (neuropeptide release in inflamed tissue) were observed (Phagoo, S. B. et al. (1999). Mol Pharmacol 56(2): 325-333; Westermann, D. et al. (2009), Diabetes 58(6): 1373-1381; Walsh, D. A. et al. (2006), Curr Drug Targets 7(8): 1031-1042; Farkas S. et al. (2011), Drugs of the Future 36(4): 301-319). Bradykinin B1 receptor agonists are endogenously produced by the activated kallikrein-kinin system. This system consists of circulating kininogens, the ubiquitous expressed proteolytic enzymes kallikreins which are activated by tissue damage, and kinins which are formed by activated kallikreins out of kininogens (Review Fincham, C. I. et al. (2009), Expert Opin Ther Pat 19(7): 919-941). These kinins (e.g. bradykinin, kalidin, des-Arg9-bradykinin, des-Arg10-kalidin) are proinflammatory peptides that mediate vascular and pain responses to tissue injury, with functions in cardiovascular homeostasis, contraction or relaxation of smooth muscle, inflammation and nociception. They exert most of their effects by interacting with two classes of G-protein-coupled receptors called Bradykinin receptor 1 and 2. The classification of the kinin receptors was originally achieved by means of pharmacological studies carried out at the end of the 1970s. During the 1990s, the existence of Bradykinin B1 receptor and B2 receptors was further confirmed through cloning and genetic deletion studies (Menke, J. G. et al. (1994), J Biol Chem 269(34): 21583-21586). The past 30 years of research on the kinin system has indicated that both Bradykinin B1 receptor and B2 receptor are involved in pain and inflammation (Leeb-Lundberg, L. M. et al. (2005), Pharmacol Rev 57(1): 27-77; Marceau, F. (2005), Trends Pharmacol Sci 26(3): 116-118; Marceau, F. (2004), Nat Rev Drug Discov 3(10): 845-852; Chen, J. J. et al. (2007), Expert Opin Ther Targets 11(1): 21-35).

It has been demonstrated that the B2 receptor is widely expressed in a constitutive manner throughout most mammalian tissues. In contrast, the Bradykinin B1 receptor is not constitutively expressed to a great extent under normal conditions, but is up-regulated under various inflammatory conditions such as asthma, arthritis and osteoarthritis, sepsis and type-1 diabetes, as well as by some neuropathological diseases such as epilepsy, stroke and multiple sclerosis. Bradykinin B1 receptor up-regulation can be induced for example by IL-1 β (Phagoo, S. B. et al. (1999), Mol Pharmacol 56(2): 325-333) and Bradykinin B2 receptor activation (NF-kB activation leading to IL-1β expression in fibroblasts) (Leeb-Lundberg, L. M. et al. (2005), Pharmacol Rev 57(1): 27-77).

Once upregulated, the Bradykinin B1 receptor is expressed on neurons, macrophages, neutrophils, fibroblasts, smooth muscle cells and the vascular endothelium (Fincham, C. I. et al. (2009), Expert Opin Ther Pat 19(7): 919-941). Recent findings suggest that the Bradykinin B1 receptor expressed in the peripheral and in the central nervous system is involved in processing of inflammatory pain (Schuelert, N. et al. (2015). Eur J Pain 19(1): 132-142).

In contrast to Bradykinin B2 receptor and many other GPCRs (G protein-coupled receptors), the Bradykinin B1 receptor does not show agonist induced internalization or relevant desensitization (Prado, G. N. et al. (2002), J Cell Physiol 193(3): 275-286; Eisenbarth, H. et al. (2004), Pain 110(1-2): 197-204). Activation of Bradykinin B1 receptor triggers auto-induction of the receptor. This might lead to an augmentation of the inflammatory or pain-inducing processes.

Therefore, Bradykinin B1 receptor has been suggested to have a pivotal role including but not limited to several chronic diseases involving diabetes, fibrosis, inflammation, neuroinflammation, neurodegeneration, inflammatory pain, and neuropathic pain (Campos, M. M. et al. (2006), Trends Pharmacol Sci 27(12): 646-651; Wang, P. H. et al. (2009), Int Immunopharmacol 9(6): 653-657; Passos, G. F. et al. (2013), Am J Pathol 182(5): 1740-1749; Gobeil, F. et al. (2014), Peptides 52: 82-89; Huart, A. (2015), Front Pharmacol 6: 8). The contribution of Bradykinin B1 receptor activation in inflammation and pain processes is supported by the demonstration that Bradykinin B1 receptor knockout mice have a largely decreased response to nociceptive and proinflammatory stimuli (Ferreira, J. et al. (2001), Neuropharmacology 41(8): 1006-1012; Ferreira, J. et al. (2005), J Neurosci 25(9): 2405-2412). The therapeutic impact of Bradykinin B1 receptor blockage for inflammation related diseases is supported further by the pharmacological properties of Bradykinin B1 receptor antagonists shown in many inflammatory and neuropathic pain models (Gougat, J. B. et al. (2004), J Pharmacol Exp Ther 309(2): 661-669; Fox, A. et al. (2005), Br J Pharmacol 144(7): 889-899).

The fact that Bradykinin B1 receptor expression is induced under disease conditions clearly raises the possibility that therapeutic use of Bradykinin B1 receptor antagonists should be devoid of undesired adverse effects. This property supports the suitability of Bradykinin B1 receptor antagonists for treatment of benign diseases like endometriosis due to the expected positive risk benefit ratio. The patient populations for nociceptive pain and neuropathic pain are large, and are driven by separate disease trends that necessitate pain relief. Chronic pain of moderate to severe intensity occurs in 19% of adult Europeans, seriously affecting the quality of their social and working lives (Breivik et al., Eur J Pain. 2006 May; 10(4):287-333). Unfortunately, current treatments for pain are only partially effective, and many cause life-style altering, debilitating, and/or dangerous side effects. For example, non-steroidal anti-inflammatory drugs (NSAIDS) such as aspirin, ibuprofen, and indomethacin are moderately effective against inflammatory pain but they are also renally toxic, and high doses tend to cause gastrointestinal irritation, ulceration, bleeding, confusion and increased cardiovascular risk. Notably, Vioxx was withdrawn from the market in 2004 due to a risk of myocardial infarction and stroke. Patients treated with opioids frequently experience confusion and constipation, and long-term opioid use is associated with tolerance and addiction. Local anaesthetics such as lidocaine and mexiletine simultaneously inhibit pain and cause loss of normal sensation. In addition, when used systemically, local anaesthetics are associated with adverse cardiovascular effects. Thus, there is currently an unmet need in the treatment of chronic pain in general.

Especially in gynaecological therapy field, endometriosis is the disease associated with chronic pelvic pain severely affecting quality of life of the patients. Globally, approximately 11% of women aged 15-49 years are affected by endometriosis and additional 6% of women suffer from symptoms suggestive for endometriosis. Main symptoms of endometriosis are chronic or frequent pelvic pain, dyspareunia, dyschezia, dysuria and sub- or infertility. These symptoms severely impair quality of life of patients. Diagnosis of the disease involves a complete medical history, a physical examination and a laparoscopy. As an ultimate confirmation of endometriosis can only be made invasively and symptoms are often unspecific, the mean time from initial symptoms to diagnosis of endometriosis is about 7-10 years. Therefore, endometriosis is under-diagnosed and the number of affected women might be much higher than anticipated. Recently published EndoCost study demonstrated that cost of productivity loss of €6,298 per woman were double the healthcare cost of €3,113 per women, driven mainly by surgery and monitoring visit (Gao, X. et al. (2006), Fertil Steril 86(6): 1561-1572; Simoens S, et al. Hum Reprod (2012), 27(5):1292-9; De Graaff A, et al. (2013), Hum Reprod; 28(10): 2677-85).

Endometriosis is characterized by growth of endometrial tissue outside of the uterine cavity forming benign tumours (lesions) in the affected part of the body. Depending on lesion location and innervation severity of pain symptoms is observed. Up-regulation of various inflammation markers observed in the affected tissue and in the peritoneal tissue underline the inflammatory character of the disease (Stratton, P. et al. (2011), Hum Reprod Update 17(3): 327-346; Gao, X. et al. (2006), Fertil Steril 86(6): 1561-1572; Laux-Biehlmann et al. (2015), Trends Pharmacol Sci 36(5): 270-276).

The Bradykinin B1 receptor was identified in endometriosis lesion by immune-histological-chemical (IHC) staining (Yoshino et al. Journal of Reproductive Immunology 112 (2015) 121-140; www.proteinatlas.org) and analysis of mRNA expression of Bradykinin B1 receptor in affected tissue shows a positive correlation to pain severity reported by endometriosis patients. Data describing a role of Bradykinin B1 receptors in affecting the outcome of an endometriosis mouse model (Jingwei, C. et al. (2015), J Tradit Chin Med 35(2): 184-191) further support the concept to treat endometriosis with Bradykinin B1 receptor antagonists.

Suspected endometriosis is initially treated with non-steroidal anti-inflammatory drugs (NSAID) or combined oral contraceptives (COC) which are used off label. This procedure delays endometriosis diagnosis. Laparoscopy is the gold standard for endometriosis diagnosis which is performed when the initial treatment options fail. During laparoscopy, endometriotic lesions are ablated. However, this procedure is accompanied by a high recurrence rate. Approximately, 70% of treated patients have persistent symptoms that are not managed. Currently, there is no long-term medication available in COC/P (Combined Oral Contraceptives/Progestin) non-responder endometriosis patients in which COCs and progestins failed. Treatment with Gonadotropin Releasing Hormone (GnRH) agonists, which are used as second line therapy (without proof of being superior versus first line) are only approved for short-term treatment (6 months). After GnRH agonist application, systemic estradiol levels are suppressed up to 90% leading to chemical castration with menopausal side effects like bone mass loss and hot flushes. Therefore, new and long-term treatment options with reduced side-effects and high efficacy for endometriosis patients, in particular for patients with COC/P non-responder endometriosis, are urgently needed.

On this background the Bradykinin B1 receptor antagonists are of value for treatment of disorders which are related to inflammation or at least partially driven by neurogenic events like diseases related to chronic pain or frequent pain conditions like but not restricted to osteoarthritis (Kaufman, G. N. et al. (2011), Arthritis Res Ther 13(3): R76), rheumatoid arthritis (Cassim, B. et al. (2009), Rheumatology 48(5): 490-496), gout (Silva, C. R. et al. (2016), Ann Rheum Dis 75(1): 260-268), burn injuries and sunburn (Eisenbarth, H. et al. (2004), Pain 110(1-2): 197-204), inflammatory bowel disease, endometriosis (Yoshino et al. Journal of Reproductive Immunology 112 (2015) 121-140; Laux-Biehlmann et al. (2015), Trends Pharmacol Sci 36(5): 270-276; Jingwei, C. et al. (2015), J Tradit Chin Med 35(2): 184-191), pre-eclampsia (Moyes, A. J. et al. (2014), Hypertens Pregnancy 33(2): 177-190), diabetic neuropathy (Dias, J. P. et al. (2007), Br J Pharmacol 152(2): 280-287) including neuropathy related to diabetes type 1 and diabetes type 2, cardiac inflammation (Westermann, D. et al. (2009), Diabetes 58(6): 1373-1381), renal inflammation (Bascands, J. et al. (2009), Biochem Biophys Res Commun 386(2): 407-412), pancreatitis and diseases related to Bradykinin B1 receptor activation and/or up-regulation in affected tissue like but not restricted to asthma and cough (Bertram, C. M. et al. (2009), J Leukoc Biol 85(3): 544-552), atherosclerosis, diabetes (Dias, J. P. et al. (2012), J Cardiovasc Pharmacol 60(1): 61-69), adipositas including metabolic syndrome (Dias, J. P. et al. (2012), Diabetes Obes Metab 14(3): 244-253), diseases related to muscle atrophy including cachexia (Parreiras, E. S. L. T. et al. (2014), Clin Sci 127(3): 185-194) not limited to cancer cachexia, neuropathic pain (Luiz, A. P. et al. (2015), Neuroscience 300: 189-200), pruritus or itch (Hosogi, M. et al. (2006), Pain 126(1-3): 16-23), cancer (da Costa, P. L. et al. (2014), Cancer Lett 345(1): 27-38), neurodegenerative diseases such as amyotrophic lateral sclerosis (ALS) or Alzheimer's disease (Lacoste, et al. (2013) J Neuroinflammation 10: 57), fibrosis in cardiacs (Westermann, D. et al. (2009), Diabetes 58(6): 1373-1381), fibrosis in renal (Huart, A. et al. (2015), Front Pharmacol 6: 8) and fibrosis in lung tissues, overactive urinary bladder syndrome and cystitis (Forner, S. et al. (2012), Br J Pharmacol 167(8): 1737-1752 and Belichard, P. et al (1999), Br J Pharmacol 128(1):213-219), impaired or painful wound healing (Schremmer-Danninger, E. et al. (2004), Biol Chem 385(11): 1069-1076) and sepsis (Murugesan, P et al. (2016), J Infect Dis 213(4): 532-540).

Several Bradykinin B1 receptor antagonists are known from prior art (Expert Opinion on Therapeutic Patents (2012), 22:12, 1443-1452). Various approaches for finding new Bradykinin B1 receptor antagonists are described, in particular peptidic structures and small molecules. Especially, arylsulfonamides and so-called cyclopropyl-carboxamides as the two main types of small molecules were investigated during the last decade.

WO2003/065789 (Merck) discloses bradykinin B1 receptor antagonists or inverse agonists of the following general formula

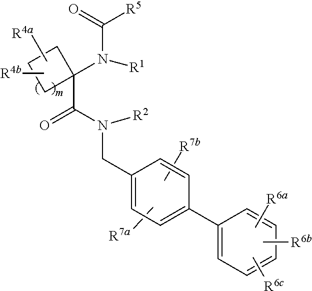

which are disclosed to be useful in the treatment or prevention of symptoms such as pain and inflammation associated with the bradykinin B1 pathway.

Merck was developing the bradykinin B1 receptor antagonist MK-0686 (structure shown below)

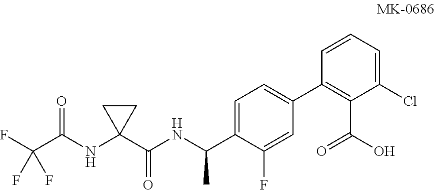 [SMILES: C[C@@H](NC(=O)C1(NC(=O)C(F)(F)F)CC1)c1ccc(-c2cccc(Cl)c2C(=O)O)cc1F]

for the potential treatment of pain and inflammation. Several phase II trials in subjects with osteoarthritis and with post-herpetic neuralgia were initiated. Merck accounted that the compound has a suboptimal pharmacokinetic profile due to metabolic liability.

Jerini AG, now Shire Group, investigated active Bradykinin B1 receptor antagonists, for example (see WO2009/036996)

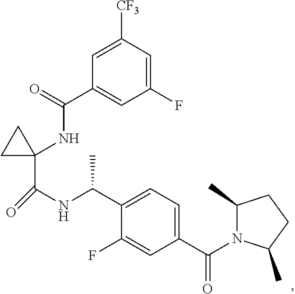 [SMILES: C[C@@H]1CC[C@H](C)N1C(=O)c1ccc([C@@H](C)NC(=O)C2(NC(=O)c3cc(F)cc(C(F)(F)F)c3)CC2)c(F)c1]

which was reported to have in addition to its activity and acceptable penetration profile reasonable aqueous solubility and pharmacokinetic profile in rat, whereas its human metabolic stability was still poor (Schaudt M, Locardi E, Zischinsky G, et al., Bioorg Med Chem Lett 2010; 20:1225-8). Jerini exchanged the cyclopropyl-carboxamide moiety to a semicarbazide or to a five-membered diamino-heterocyclic ring or even to hydroxyureas without any explanation.

Starting with arylsulfonamide compounds as Bradykinin B1 receptor antagonists, Boehringer Ingelheim reported about several cyclopropyl-carboxamides out of their further development compounds like of the following structure

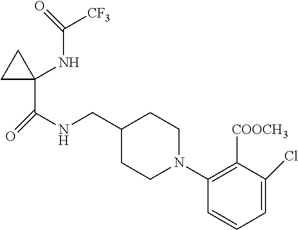 [SMILES: COC(=O)c1c(Cl)cccc1N1CCC(CNC(=O)C2(NC(=O)C(F)(F)F)CC2)CC1]

or related to that emerged with the highest binding affinity measured on human B1R-expressing CHO cells (Expert Opinion on Therapeutic Patents (2012), 22:12, 1443-1452).

In WO2012/059776 Gedeon Richter reported about cyclopropyl-carboxamides of the following formula

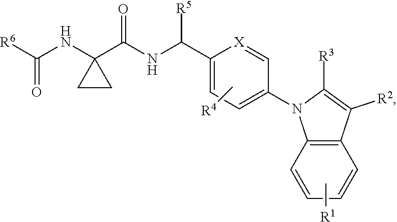

wherein R³ is selected from (1) —COOR; (2) —CN; (3) —CONR^{a}R^{b};

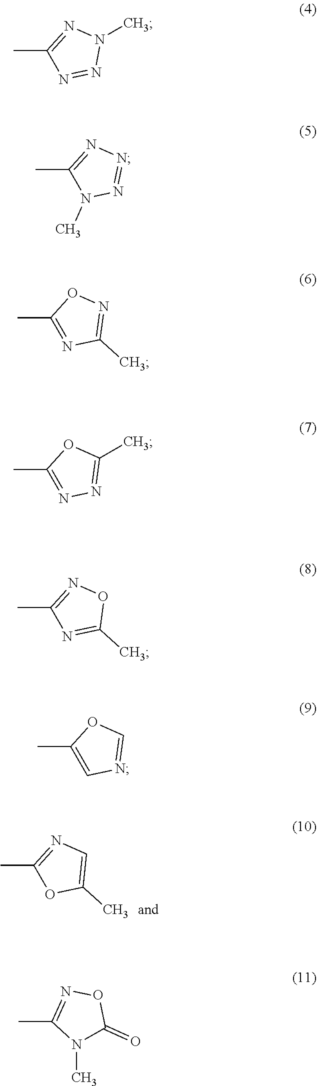

A majority of the compounds have a Kᵢ value below 20 nM on human recombinant Bradykinin receptors (expressed in CHO cells). Several indolyl compounds are disclosed and represented by the following compound:

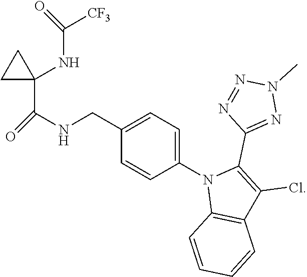 [SMILES: Cn1nnc(-c2c(Cl)c3ccccc3n2-c2ccc(CNC(=O)C3(NC(=O)C(F)(F)F)CC3)cc2)n1]

WO2005085227 (Smith Kline Beecham) discloses inhibitors of protein kinase B (PKB/Akt, PKB or Akt) of the formula

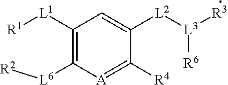

wherein
A is selected from: nitrogen, —C-halogen and —CH;
R¹ is selected from the group consisting of aryl, substituted aryl, cycloalkyl, substituted cycloalkyl, heterocycle and substituted heterocycle;
R² is selected from alkyl, substituted alkyl, cycloalkyl, substituted cycloalkyl, heterocycle, substituted heterocycle, and a cyclic or polycyclic aromatic ring,
L² is selected from the group consisting of a bond, —O—, heterocycle, —N(R⁵)—, —N(R⁵)C(O)—, —S—, —S(O)—, —S(O₂)—, and —C(O)N(R⁵)—; and
L¹ as well as L⁶ can be a bond, —O—, —N(R⁵)—, —S—, —S(O), —S(O₂)—, alkyl, and —N(R⁵)C(O)—. Neither L¹ nor L⁶ can be a heteroaryl or heterocyclic group. R⁴ is defined as hydrogen or halogen. The compounds are disclosed to be suitable for the treatment of cancer and arthritis. Tetrazole-substituted phenyl or pyridinyl compounds are not specifically disclosed.

In WO2012/112567 (Georgetown University) small molecule inhibitors of ATP/GTP binding protein like 2 (AGBL2) of the formula

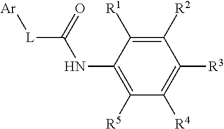

are disclosed wherein Ar is substituted or unsubstituted aryl or substituted or unsubstituted heteroaryl; L is absent or —(CHR⁶)—, wherein R⁶ is substituted or unsubstituted alkyl, substituted or unsubstituted alkenyl, substituted or unsubstituted alkynyl, substituted or unsubstituted cycloalkyl, substituted or unsubstituted heterocycloalkyl, substituted or unsubstituted aryl, or substituted or unsubstituted heteroaryl; and R¹, R², R³, R⁴, and R⁵ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, substituted or unsubstituted carbonyl, or substituted or unsubstituted carboxyl. The compounds are disclosed to be used in methods for treating or preventing cancer and neurologic disorders.

WO2012/103583 (Bionomics) discloses 1,2-cyclopropyl-carboxamide compounds of general formula (I)

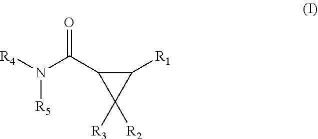

wherein R⁴ is selected from optionally substituted heteroaryl, optionally substituted heterocyclyl, or optionally substituted aryl, and R⁵ is selected from hydrogen or optionally substituted alkyl. Such compounds are disclosed to be useful in the positive modulation of the alpha 7 nicotinic acetylcholine receptor (alpha7nAChR). The disclosure of WO2012103583 also relates to the use of these compounds in the treatment or prevention of a broad range of diseases in which the positive modulation of alpha7nAChR is advantageous, including neurodegenerative and neuropsychiatric diseases and inflammatory diseases. 1,1-cyclopropyl-carboxamide compounds are not disclosed.

WO2007/087066 (Vertex) discloses compounds and pharmaceutically acceptable compositions thereof, which are disclosed to be useful as modulators of ATP-Binding Cassette (“ABC”) transporters or fragments thereof, including Cystic Fibrosis Transmembrane Conductance Regulator (“CFTR”), having a benzamide core structure (I)

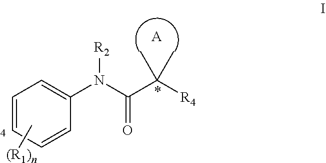

wherein ring A is an optionally substituted cycloaliphatic or an optionally substituted heterocycloaliphatic where the atoms of ring A adjacent to C* are carbon atoms. R₄ is an optionally substituted aryl or an optionally substituted heteroaryl. R¹ is independently an optionally substituted C₁-C₆ aliphatic, an optionally substituted aryl, an optionally substituted heteroaryl, an optionally substituted C₃-C₁₀ membered cycloaliphatic or an optionally substituted 4 to 10 membered heterocycloaliphatic, carboxy, amido, amino, halo, or hydroxy, provided that at least one R¹ is an optionally substituted aryl or an optionally substituted heteroaryl and said R¹ is attached to the 3- or 4-position of the phenyl ring. The compounds of the present invention are not disclosed.

So, the state of the art described above does not describe the compounds of general formula (I) of the present invention as defined herein or an isomer, enantiomer, diastereomer, racemate, hydrate, solvate, or a salt thereof, or a mixture of same, as described and defined herein, and as hereinafter referred to as “compounds of the present invention”, or their pharmacological activity.

### SUMMARY OF THE INVENTION

The present invention covers compounds of general formula (I)

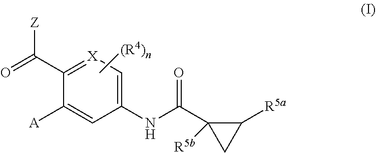

in which
A represents tetrazolyl which is attached to the rest of the molecule via the carbon atom;
X represents CH, CR⁶ or N;
Z represents OR¹ or NR²R³;
R¹ represents C₁-C₇-alkyl, C₃-C₇-cycloalkyl, 4- to 7-membered heterocycloalkyl, (4- to 7-membered heterocycloalkyl)-C₁-C₄-alkyl, phenyl or 5- or 6-membered heteroaryl, wherein said C₁-C₇-alkyl is optionally substituted, one or more times, independently from each other, with C₁-C₄-alkoxy and said C₃-C₇-cycloalkyl, 4- to 7-membered heterocycloalkyl, (4- to 7-membered heterocycloalkyl)-C₁-C₄-alkyl, phenyl or 5- or 6-membered heteroaryl groups are optionally substituted, one or more times, independently from each other, with C₁-C₄-alkyl or C₁-C₄-alkoxy;
R², R³ represent, independently from each other, hydrogen, C₁-C₇-alkyl, C₃-C₇-cycloalkyl, 4- to 7-membered heterocycloalkyl, C₃-C₇-cycloalkyl-C₁-C₄-alkyl, (4- to 7-membered heterocycloalkyl)-C₁-C₄-alkyl, phenyl or 5- or 6-membered heteroaryl, wherein said C₁-C₇-alkyl is optionally substituted, one or more times, independently from each other, with C₁-C₄-alkoxy and said C₃-C₇-cycloalkyl, 4- to 7-membered heterocycloalkyl, C₃-C₇-cycloalkyl-C₁-C₄-alkyl, (4- to 7-membered heterocycloalkyl)-C₁-C₄-alkyl, phenyl or 5- or 6-membered heteroaryl groups are optionally substituted, one or more times, independently from each other, with C₁-C₄-alkyl or C₁-C₄-alkoxy, or
R² and R³ together with the nitrogen atom to which they are attached form a 5- to 6-membered heterocyclic ring, optionally containing one additional heteroatom selected from O and N and optionally substituted with C₁-C₄-alkyl or C₁-C₄-alkoxy;
R⁴ represents C₁-C₄-alkyl or halogen;
R^{5a} represents hydrogen, phenyl or heteroaryl;
   wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy;
R^{5b} represents hydrogen, phenyl or heteroaryl;
   wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy and wherein one of R^{5a} and R^{5b} represents hydrogen and the other represents phenyl or heteroaryl;
R⁶ represents C₁-C₄-alkyl or halogen; and
n represents 0, 1, or 2;
   and stereoisomers, tautomers, N-oxides, hydrates, solvates, and salts thereof, and mixtures of same.

The present invention further relates to pharmaceutical compositions and combinations comprising said compounds, to use of said compounds for manufacturing a medicament for the treatment or prophylaxis of diseases or disorders and for the treatment of pains, which are associated with such diseases.

### DETAILED DESCRIPTION OF THE INVENTION

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or sulfur atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “one or a plurality up to the maximum possible amount”, e.g. if the term refers to the carbon atoms of a C₇-cycloalkyl, it relates to “1, 2, 3, 4, 5, 6 or 7”. In particular, “one or more” means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups, which occur repeatedly, are independent from one another. It is possible that groups in the compounds according to the invention are substituted with one, two or three identical or different substituents, particularly with one substituent.

The term “comprising” when used in the specification includes but is not restricted to “consisting of”.

The terms as mentioned in the present text have preferably the following meanings:

The term “halogen atom”, “halogen”, “halo-” or “Hal-” is to be understood as meaning a fluorine, chlorine, bromine or iodine atom, preferably a fluorine or a chlorine atom.

The term “C₁-C₅-alkyl” means a linear or branched, saturated, monovalent hydrocarbon group having 1, 2, 3, 4 or 5 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl or 1,1-dimethylpropyl group, or an isomer thereof.

The term “C₁-C₃-alkyl” means a linear or branched, saturated, monovalent hydrocarbon group having 1, 2 or 3 carbon atoms (“C₁-C₃-alkyl”), e.g. a methyl, ethyl, n-propyl or isopropyl group.

The term “—OC₁-C₅-alkyl” means a linear or branched, saturated, monovalent group which is attached through an oxygen atom, and in which the term “C₁-C₅-alkyl” is as defined supra, e.g. a methoxy, ethoxy, n-propoxy, isopropoxy, n-butoxy, sec-butoxy, isobutoxy, tert-butoxy, pentyloxy or isopentyloxy, or an isomer thereof. The hyphen at the beginning of the group indicates the point of attachment of said OC₁-C₅-alkyl group to the rest of the molecule.

The term “C₁-C₄-alkoxy” means a linear or branched, saturated, monovalent group of formula (C₁-C₄-alkyl)-O—, in which the term “C₁-C₄-alkyl” is as defined supra, e.g. a methoxy, ethoxy, n-propoxy, isopropoxy, n-butoxy, sec-butoxy, isobutoxy, or tert-butoxy group, or an isomer thereof.

The term “C₁-C₄-haloalkyl” means a linear or branched, saturated, monovalent hydrocarbon group in which the term “C₁-C₄-alkyl” is as defined supra, and in which one or more of the hydrogen atoms are replaced, identically or differently, with a halogen atom. Particularly, said halogen atom is a fluorine atom. Said C₁-C₄-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₄-haloalkoxy” means a linear or branched, saturated, monovalent C₁-C₄-alkoxy group, as defined supra, in which one or more of the hydrogen atoms is replaced, identically or differently, with a halogen atom. Particularly, said halogen atom is a fluorine atom. Said C₁-C₄-haloalkoxy group is, for example, fluoromethoxy, difluoromethoxy, trifluoromethoxy, 2,2,2-trifluoroethoxy or pentafluoroethoxy.

“C₃-C₇-cycloalkyl” is to be understood as meaning a saturated, monovalent, monocyclic or bicyclic hydrocarbon ring, which contains 3, 4, 5, 6 or 7 carbon atoms. Said C₃-C₇-cycloalkyl group is for example a monocyclic hydrocarbon ring, e.g. a cyclopropyl, cyclobutyl, cyclopentyl, cyclohexyl or cycloheptyl group, or a bicyclic hydrocarbon ring, e.g. a bicyclo[2.2.1]heptanyl or bicyclo[3.2.0]heptanyl group. Particularly, said ring contains 3, 4 or 5 carbon atoms (“C₃-C₅-cycloalkyl”) or 5, 6 or 7 carbon atoms (“C₅-C₇-cycloalkyl”).

The term “bicyclic cycloalkyl” includes by definition spirocycloalkyl, bridged, and fused bicycloalkyl groups.

The term “spirocycloalkyl” means a saturated, monovalent bicyclic hydrocarbon group in which the two rings share one common ring carbon atom, and wherein said bicyclic hydrocarbon group contains 5, 6, or 7 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl or spiro[2.4]heptyl.

The term “fused bicycloalkyl” means a bicyclic, saturated hydrocarbon ring with 6 or 7 ring atoms in total, in which the two rings share two adjacent ring atoms.

Said fused cycloalkyl group is, for example, a bicyclo[3.1.0]hexanyl or bicyclo[3.2.0]heptanyl group.

The term “bridged bicycloalkyl” means a bicyclic, saturated hydrocarbon ring with 6 or 7 ring atoms in total, in which the two rings share two common ring atoms which are not adjacent. Said bridged cycloalkyl group is, for example, bicyclo[2.1.1]hexanyl or bicyclo[2.2.1]heptanyl group.

The term “—(C₁-C₃-alkyl)-(C₃-C₇-cycloalkyl)” is to be understood as a C₃-C₇-cycloalkyl group as defined above which is attached through any carbon atom of said C₃-C₇-cycloalkyl group to any atom of the C₁-C₃-alkyl group as defined above. The hyphen at the beginning of the group indicates the point of attachment of said (C₁-C₃-alkyl)-(C₃-C₇-cycloalkyl) group to the rest of the molecule. Said (C₁-C₃-alkyl)-(C₃-C₇-cycloalkyl) groups are, for example, cyclopropylmethyl, cyclobutylmethyl, cyclopentylmethyl, cyclohexylmethyl, 2-cyclopropylethyl, 1-cyclopropylethyl, 2-cyclobutylethyl, 1-cyclobutylethyl, 2-cyclopentylethyl, 1-cyclopentylethyl, 2-cyclobutylpropyl, or 1-cyclobutylpropyl.

The term “—OC₃-C₇-cycloalkyl” means a saturated, monovalent, monocyclic group, which contains 3, 4, 5, 6 or 7 carbon atoms, in which the term “C₃-C₇-cycloalkyl” is defined supra, e.g. a cyclopropyloxy, cyclobutyloxy, cyclopentyloxy, cyclohexyloxy, or cycloheptyloxy group.

The term “heterocycloalkyl” is to be understood as meaning a saturated, monovalent, monocyclic or bicyclic hydrocarbon ring with the number of ring atoms as specified in which one or two ring atoms of the hydrocarbon ring is/are replaced by one or two heteroatoms or heteroatom-containing groups independently selected from NH, —NR⁷, N, O, S, SO and SO₂, wherein R⁷ represents C₁-C₅-alkyl optionally substituted with 1 to 5 fluorine atoms.

4- to 7-membered heterocycloalkyl in the context of the invention means a monocyclic or bicyclic, saturated heterocycle with 4, 5, 6 or 7 ring atoms in total, which contains one or two identical or different ring heteroatoms or heteroatom-containing groups from the series NH, —NR⁷, N, O, S, SO and SO₂, wherein R⁷ represents C₁-C₅-alkyl optionally substituted with 1 to 5 fluorine atoms. Said 4- to 7-membered heterocycloalkyl can be bound via a ring carbon or nitrogen atom to the rest of the molecule.

Said heterocycloalkyl can be connected to the rest of the molecule through a carbon or a nitrogen atom, if said nitrogen atom is present.

Examples for monocyclic heterocycloalkyl groups are azetidinyl, oxetanyl, thietanyl, pyrrolidinyl, pyrazolidinyl, imidazolidinyl, tetrahydrofuranyl, thiolanyl, 1,1-dioxidothiolanyl, 1,2-oxazolidinyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, 1,2-oxazinanyl, morpholinyl, thiomorpholinyl, 1,1-dioxidothiomorpholinyl, azepanyl, 1,4-diazepanyl, and 1,4-oxazepanyl.

Particularly, without being limited thereto, said heterocycloalkyl can be a 4-membered ring, such as an azetidinyl, oxetanyl or thietanyl, or a 5-membered ring, such as tetrahydrofuranyl, dioxolinyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, or a 6-membered ring such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, or a 7-membered ring, such as a azepanyl, 1,4-diazepanyl, or 1,4-oxazepanyl, for example.

The term “bicyclic heterocycloalkyl” includes by definition heterospirocycloalkyl, fused and bridged heterobicycloalkyl groups.

The term “heterospirocycloalkyl” means a bicyclic, saturated heterocycle with 6 or 7 ring atoms in total, in which the two rings share one common ring carbon atom, wherein the “heterospirocycloalkyl” contains one or two identical or different ring heteroatoms or heteroatom-containing groups from the series: NH, —NR⁷, N, O, S, SO and SO₂, wherein R⁷ represents C₁-C₅-alkyl optionally substituted with 1 to 5 fluorine atoms; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[2.4]heptanyl, azaspiro[3.3]heptyl, oxazaspiro[3.3]heptyl, thiazaspiro[3.3]heptyl, oxaspiro[3.3]heptyl, diazaspiro[3.3]heptyl or thiazaspiro[3.3]heptyl, or one of the further homologous scaffolds such as spiro[2.3]-, spiro[2.4]-, spiro[3.3]-.

The term “fused heterocycloalkyl” means a bicyclic, saturated heterocycle with 6 or 7 ring atoms in total, in which the two rings share two adjacent ring atoms, which “fused heterocycloalkyl” contains one or two identical or different ring heteroatoms or heteroatom-containing groups from the series: NH, —NR⁷, N, O, S, SO and SO₂, wherein R⁷ represents C₁-C₅-alkyl optionally substituted with 1 to 5 fluorine atoms; it being possible for said fused heterocycloalkyl group to be attached to the rest of the molecule via any one of the carbon atoms or, if present, a nitrogen atom.

Said fused heterocycloalkyl group is, for example, 3-azabicyclo[3.1.0]hexanyl or 3-azabicyclo[3.2.0]heptanyl.

The term “bridged heterocycloalkyl” means a bicyclic, saturated heterocycle with 6 or 7 ring atoms in total, in which the two rings share two common ring atoms which are not adjacent, which “bridged heterocycloalkyl” contains one or two identical or different ring heteroatoms or heteroatom-containing groups from the series: NH, —NR⁷, N, O, S, SO and SO₂, wherein R⁷ represents C₁-C₅-alkyl optionally substituted with 1 to 5 fluorine atoms; it being possible for said bridged heterocycloalkyl group to be attached to the rest of the molecule via any one of the carbon atoms, except the bridgehead carbon atoms, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, or diazabicyclo[2.2.1]heptyl.

The term “5- to 7-membered lactam” means cyclic amides of amino carboxylic acids, having a 1-azacycloalkan-2-one structure, or analogues having unsaturation or heteroatoms replacing one or more carbon atoms of the ring having a ring size of 5, 6 or 7 ring system atoms. In particular said “5- to 7-membered lactam” means a γ-lactam (gamma-lactam), a δ-lactam (delta-lactam), and an ε-lactam (epsilon-lactam).

The term “heteroaryl” is understood as meaning a monovalent, monocyclic or bicyclic hydrocarbon ring system with at least one aromatic ring, and wherein at least one ring atom of the monovalent, monocyclic or bicyclic hydrocarbon ring system can be replaced by at least one heteroatom or heteroatom-containing group, like NH, N, O, S, SO, and SO₂. The number of ring system atoms is as specified, e.g. a 5- or 6-membered heteroaryl.

“5- or 6-membered heteroaryl” is understood as meaning a monovalent, monocyclic heteroaryl having 5 or 6 ring atoms and wherein one, two or three ring atoms of a monovalent 5-membered hydrocarbon ring system is/are replaced by one, two or three heteroatoms or heteroatom-containing groups independently selected from S, N, NH and O; and wherein one or two ring atoms of a monovalent 6-membered hydrocarbon ring system is/are replaced by one or two nitrogen atoms.

The said 5-membered heteroaryl can be connected through a carbon or a nitrogen atom, if said nitrogen atom is present.

Said 5- or 6-membered heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl or thiadiazolyl; or a 6-membered heteroaryl group, such as, for example, pyridinyl, pyridazinyl, pyrimidinyl or pyrazinyl.

In general, and unless otherwise mentioned, the term “heteroaryl” includes all possible isomeric forms thereof, e.g. tautomers and positional isomers with respect to the point of linkage to the rest of the molecule. Thus, to give some illustrative non-restricting examples, the term pyridinyl includes pyridin-2-yl, pyridin-3-yl, and pyridin-4-yl; or the term pyrimidinyl includes pyrimidin-2-yl, pyrimidin-4-yl and pyrimidin-5-yl; or the term pyrazolyl includes 1H-pyrazolyl; or the term imidazolyl includes 1H-imidazolyl and 4H-imidazolyl; the term thiophenyl includes 2-thiophenyl and 3-thiophenyl; or the term thiazolyl includes 1,3-thiazol-5-yl, 1,3-thiazol-4-yl and 1,3-thiazol-2-yl.

“Bicyclic 8- to 10-membered heteroaryl” is understood as meaning a bicyclic, monovalent, fused heteroaryl having 8, 9 or 10 ring atoms with at least one aromatic ring and wherein one, two or three ring atoms of a monovalent, 8- to 10-membered bicyclic hydrocarbon ring system is/are replaced by one, two or three heteroatoms or heteroatom-containing groups independently selected from NH, N, O, S, SO and SO₂.

The said bicyclic 8- to 10-membered heteroaryl can be connected through a carbon or a nitrogen atom, if said nitrogen atom is present.

The term “bicyclic 8- to 10-membered heteroaryl” includes by definition fused and bridged heterobicycloalkyl groups.

Particularly, bicyclic heteroaryl is selected from for example, benzofuranyl, benzothienyl, benzothiazolyl, thienopyridinyl, thienopyrimidinyl, benzoxazolyl, benzisoxazolyl, benzimidazolyl, benzotriazolyl, benzothiadiazolyl, indazolyl, indolyl, isoindolyl, etc. or for example, quinolinyl, quinazolinyl, isoquinolinyl, etc.; indolizinyl, or cinnolinyl, phthalazinyl, quinazolinyl, quinoxalinyl, etc.

The term “C₁-C₃” as used throughout this text is to be understood as meaning a group having a finite number of carbon atoms of 1 to 3, i.e. 1, 2, or 3 carbon atoms, e.g. in the context of the definition of “C₁-C₃-alkyl”, it is to be understood as meaning an alkyl group having a finite number of carbon atoms of 1 to 3, i.e. 1, 2, or 3 carbon atoms. It is to be understood further that said term “C₁-C₃” is to be interpreted as any sub-range comprised therein, e.g. C₁-C₂, or C₂-C₃.

The term “C₁-C₅” as used throughout this text is to be understood as meaning a group having a finite number of carbon atoms of 1 to 5, i.e. 1, 2, 3, 4, or 5 carbon atoms, e.g. in the context of the definition of “C₁-C₅-alkyl”, it is to be understood as meaning an alkyl group having a finite number of carbon atoms of 1 to 5, i.e. 1, 2, 3, 4, or 5 carbon atoms. It is to be understood further that said term “C₁-C₅” is to be interpreted as any sub-range comprised therein, e.g. C₁-C₅, C₂-C₅, C₃-C₄, C₂-C₃, C₂-C₄, or C₁-C₄.

The term “C₁-C₃” as used in the context of the definition “—OC₁-C₃-alkyl” is to be understood as meaning an alkyl group, having a finite number of carbon atoms of 1 to 3, i.e. 1, 2 or 3 carbon atoms.

Similarly, the mentioned above applies to “C₁-C₄-alkyl”, “C₁-C₃-alkyl”, “C₁-C₃-alkoxy”, “C₁-C₂-alkyl” or “C₁-C₂-alkoxy”.

Further, as used herein, the term “C₃-C₇”, as used throughout this text, is to be understood as meaning a group having a finite number of carbon atoms of 3 to 7, i.e. 3, 4, 5, 6 or 7 carbon atoms, e.g. in the context of the definition of “C₃-C₇-cycloalkyl”, it is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, or C₅-C₇; particularly C₃-C₆.

Furthermore, as used herein, the term “C₃-C₅”, as used in the present text, e.g. in the context of the definition of “C₃-C₅-cycloalkyl”, means a cycloalkyl group having a finite number of carbon atoms of 3 to 5, i.e. 3, 4 or 5 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-05 and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methyl sulfonyl)oxy, [(trifluoromethyl)sulfonyl]oxy, [(nonafluorobutyl)sulfonyl]oxy, (phenyl sulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)-sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope, which is higher than its natural abundance. The natural abundances of isotopes to be applied in this context are described in “Isotopic Compositions of the Elements 1997”, Pure Appl. Chem., 70(1), 217-235, 1998, which is incorporated herein by reference.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271] and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases, deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102; both incorporated herein by reference). In other cases, the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208; incorporated herein by reference) and Odanacatib (K. Kassahun et al., WO2012/112363; incorporated herein by reference) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993; incorporated herein by reference). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976), thereby incorporated herein.

Further, the compounds of the present invention may exist as tautomers.

The present invention includes all possible tautomers of the compounds of the present invention as single tautomers, or as any mixture of said tautomers, in any ratio.

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example. The 1,2-cyclopropylamides of the invention have to be understood, unless stated otherwise, as relating to both cis and trans isomers, as either single entantiomers or a mixture of enantiomers. Preferred are mixtures of trans enantiomers, if not stated otherwise.

The present invention also relates to useful forms of the compounds as disclosed herein, such as hydrates, solvates, and salts, in particular pharmaceutically acceptable salts.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19, incorporated herein by reference. A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecyl sulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemi sulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Unless otherwise indicated, the compounds of the present invention are also referred to isomers, enantiomers, diastereomers, racemates, hydrates, solvates, a salt thereof, or a mixture of same.

As used herein, the term “in vivo hydrolysable ester” is understood as meaning an in vivo hydrolysable ester of a compound of the present invention containing a carboxy or hydroxy group, for example, a pharmaceutically acceptable ester that is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention. An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorph, in any ratio.

In accordance with a preferred embodiment, the present invention covers compounds of general formula (I), supra, in which:
A represents tetrazolyl which is attached to the rest of the molecule via the carbon atom;
X represents CH, CR⁶ or N;
Z represents OR¹ or NR²R³;
R¹ represents C₁-C₆-alkyl, C₃-C₆-cycloalkyl, 4- to 6-membered heterocycloalkyl, (4- to 6-membered heterocycloalkyl)-C₁-C₂-alkyl, phenyl or 5- or 6-membered heteroaryl,
   wherein said C₁-C₆-alkyl is optionally substituted, one or more times, independently from each other, with C₁-C₃-alkoxy
   and said C₃-C₆-cycloalkyl, 4- to 6-membered heterocycloalkyl, (4- to 6-membered heterocycloalkyl)-C₁-C₂-alkyl, phenyl or 5- or 6-membered heteroaryl groups are optionally substituted, one or more times, independently from each other, with C₁-C₃-alkyl or C₁-C₃-alkoxy;
R², R³ represent, independently from each other, hydrogen, C₁-C₆-alkyl, C₃-C₆-cycloalkyl, 4- to 6-membered heterocycloalkyl, C₃-C₆-cycloalkyl-C₁-C₂-alkyl, (4- to 6-membered heterocycloalkyl)-C₁-C₂-alkyl, phenyl or 5- or 6-membered heteroaryl,
   wherein said C₁-C₆-alkyl is optionally substituted, one or more times, independently from each other, with C₁-C₃-alkoxy
   and said C₃-C₆-cycloalkyl, 4- to 6-membered heterocycloalkyl, C₃-C₆-cycloalkyl-C₁-C₂-alkyl, (4- to 6-membered heterocycloalkyl)-C₁-C₂-alkyl, phenyl or 5- or 6-membered heteroaryl groups are optionally substituted, one or more times, independently from each other, with C₁-C₃-alkyl or C₁-C₃-alkoxy, or
R² and R³ together with the nitrogen atom to which they are attached form a 5- to 6-membered heterocycloalkyl, optionally containing one additional heteroatom selected from O and N and optionally substituted with C₁-C₃-alkyl or C₁-C₃-alkoxy;
R⁴ represents C₁-C₃-alkyl or halogen;
R^{5a} represents hydrogen or phenyl;
   wherein said phenyl group is optionally substituted, one or more times, independently from each other, with halogen, C₁-C₄-alkyl, C₁-C₄-alkoxy, C₁-C₄-haloalkyl or C₁-C₄-haloalkoxy;
R^{5b} represents hydrogen or phenyl;
   wherein said phenyl group is optionally substituted, one or more times, independently from each other, with halogen, C₁-C₄-alkyl, C₁-C₄-alkoxy, C₁-C₄-haloalkyl or C₁-C₄-haloalkoxy and wherein one of R^{5a} and R^{5b} represents hydrogen and the other represents phenyl;
R⁶ represents C₁-C₃-alkyl or halogen; and
n represents 0, or 1;
   and stereoisomers, tautomers, N-oxides, hydrates, solvates, and salts thereof, and mixtures of same.

In accordance with a preferred embodiment, the present invention covers compounds of general formula (I) in which:
A represents tetrazolyl which is attached to the rest of the molecule via the carbon atom;
X represents CH or CR⁶;
Z represents OR¹ or NR²R³;
R¹ represents C₂-C₅-alkyl, C₄-C₆-cycloalkyl, 5- to 6-membered heterocycloalkyl, or tetrahydro-2H-pyran-4-yl-methyl,
   wherein said C₂-C₅-alkyl is optionally substituted with methoxy and said C₄-C₆-cycloalkyl and 5- to 6-membered heterocycloalkyl groups are optionally substituted with methyl;
R², R³ represent, independently from each other, hydrogen, C₁-C₄-alkyl, C₅-C₆-cycloalkyl, 5- to 6-membered heterocycloalkyl, C₅-C₆-cycloalkyl-methyl, tetrahydro-2H-pyran-4-yl-methyl or pyridyl,
   wherein said C₁-C₄-alkyl is optionally substituted with methoxy and said pyridyl groups are optionally substituted with methyl, or
R² and R³ together with the nitrogen atom to which they are attached form a 6-membered heterocycloalkyl, optionally substituted with methoxy;
R⁴ represents methyl or fluoro;
R^{5a} represents hydrogen or phenyl;
   wherein said phenyl group is optionally substituted with methyl;
R^{5b} represents hydrogen or phenyl;
   wherein said phenyl group is optionally substituted, one or two times, independently from each other, with fluoro, chloro, methyl, trifluoromethyl or trifluoromethoxy and wherein one of R^{5a} and R^{5b} represents hydrogen and the other represents phenyl;
R⁶ represents C₁-C₃-alkyl or halogen; and
n represents 0 or 1.

In any embodiment of the invention, the present invention covers compounds of general formula (I) in which A represents tetrazolyl which is attached to the rest of the molecule via the carbon atom.

In a preferred embodiment, the present invention covers compounds of general formula (I) in which X represents CH or N.

In a preferred embodiment, the present invention covers compounds of general formula (I) in which X represents CH.

In a preferred embodiment, the present invention covers compounds of general formula (I) in which X represents N.

In a preferred embodiment, the present invention covers compounds of general formula (I) in which Z represents OR¹ or NR²R³.

In a preferred embodiment, the present invention covers compounds of general formula (I) in which Z represents OR¹.

In a preferred embodiment, the present invention covers compounds of general formula (I) in which Z represents NR²R³.

In a preferred embodiment, the present invention covers compounds of general formula (I) in which R¹ represents C₁-C₇-alkyl, C₃-C₇-cycloalkyl, 4- to 7-membered heterocycloalkyl, (4- to 7-membered heterocycloalkyl)-C₁-C₄-alkyl, phenyl or 5- or 6-membered heteroaryl, wherein said C₁-C₇-alkyl is optionally substituted, one or more times, independently from each other, with C₁-C₄-alkoxy and said C₃-C₇-cycloalkyl, 4- to 7-membered heterocycloalkyl, (4- to 7-membered heterocycloalkyl)-C₁-C₄-alkyl, phenyl or 5- or 6-membered heteroaryl groups are optionally substituted, one or more times, independently from each other, with C₁-C₄-alkyl or C₁-C₄-alkoxy.

In a preferred embodiment, the present invention covers compounds of general formula (I) in which:
R¹ represents C₁-C₆-alkyl, C₃-C₆-cycloalkyl, 4- to 6-membered heterocycloalkyl, (4- to 6-membered heterocycloalkyl)-C₁-C₂-alkyl, phenyl or 5- or 6-membered heteroaryl,
   wherein said C₁-C₆-alkyl is optionally substituted, one or more times, independently from each other, with C₁-C₃-alkoxy and said C₃-C₆-cycloalkyl, 4- to 6-membered heterocycloalkyl, (4- to 6-membered heterocycloalkyl)-C₁-C₂-alkyl, phenyl or 5- or 6-membered heteroaryl groups are optionally substituted, one or more times, independently from each other, with C₁-C₃-alkyl or C₁-C₃-alkoxy.

In a preferred embodiment, the present invention covers compounds of general formula (I) in which:
R¹ represents C₂-C₅-alkyl, C₄-C₆-cycloalkyl, 5- to 6-membered heterocycloalkyl, or tetrahydro-2H-pyran-4-yl-methyl,
   wherein said C₂-C₅-alkyl is optionally substituted with methoxy and said C₄-C₆-cycloalkyl and 5- to 6-membered heterocycloalkyl groups are optionally substituted with methyl.

In a preferred embodiment of, the present invention covers compounds of general formula (I) in which:
R², R³ represent, independently from each other, hydrogen, C₁-C₇-alkyl, C₃-C₇-cycloalkyl, 4- to 7-membered heterocycloalkyl, C₃-C₇-cycloalkyl-C₁-C₄-alkyl, (4- to 7-membered heterocycloalkyl)-C₁-C₄-alkyl, phenyl or 5- or 6-membered heteroaryl,
   wherein said C₁-C₇-alkyl is optionally substituted, one or more times, independently from each other, with C₁-C₄-alkoxy and said C₃-C₇-cycloalkyl, 4- to 7-membered heterocycloalkyl, C₃-C₇-cycloalkyl-C₁-C₄-alkyl, (4- to 7-membered heterocycloalkyl)-C₁-C₄-alkyl, phenyl or 5- or 6-membered heteroaryl groups are optionally substituted, one or more times, independently from each other, with C₁-C₄-alkyl or C₁-C₄-alkoxy.

In a preferred embodiment, the present invention covers compounds of general formula (I) in which:
R² and R³ together with the nitrogen atom to which they are attached form a 5- to 6-membered heterocycloalkyl, optionally containing one additional heteroatom selected from O and N and optionally substituted with C₁-C₄-alkyl or C₁-C₄-alkoxy.

In a preferred embodiment, the present invention covers compounds of general formula (I) in which:
R², R³ represent, independently from each other, hydrogen, C₁-C₆-alkyl, C₃-C₆-cycloalkyl, 4- to 6-membered heterocycloalkyl, C₃-C₆-cycloalkyl-C₁-C₂-alkyl, (4- to 6-membered heterocycloalkyl)-C₁-C₂-alkyl, phenyl or 5- or 6-membered heteroaryl,
   wherein said C₁-C₆-alkyl is optionally substituted, one or more times, independently from each other, with C₁-C₃-alkoxy and said C₃-C₆-cycloalkyl, 4- to 6-membered heterocycloalkyl, C₃-C₆-cycloalkyl-C₁-C₂-alkyl, (4- to 6-membered heterocycloalkyl)-C₁-C₂-alkyl, phenyl or 5- or 6-membered heteroaryl groups are optionally substituted, one or more times, independently from each other, with C₁-C₃-alkyl or C₁-C₃-alkoxy.

In a preferred embodiment, the present invention covers compounds of general formula (I) in which:
R² and R³ together with the nitrogen atom to which they are attached form a 5- to 6-membered heterocycloalkyl, optionally containing one additional heteroatom selected from O and N and optionally substituted with C₁-C₃-alkyl or C₁-C₃-alkoxy.

In a preferred embodiment, the present invention covers compounds of general formula (I) in which:
R², R³ represent, independently from each other, hydrogen, C₁-C₄-alkyl, C₅-C₆-cycloalkyl, 5- to 6-membered heterocycloalkyl, C₅-C₆-cycloalkyl-methyl, (tetrahydro-2H-pyran-4-yl-methyl or pyridyl,
   wherein said C₁-C₄-alkyl is optionally substituted with methoxy and said pyridyl groups are optionally substituted with methyl.

In a preferred embodiment, the present invention covers compounds of general formula (I) in which R² and R³ together with the nitrogen atom to which they are attached form a 6-membered heterocycloalkyl, optionally substituted with methoxy.

In a preferred embodiment, the present invention covers compounds of general formula (I) in which R⁴ represents C₁-C₄-alkyl or halogen.

In a preferred embodiment, the present invention covers compounds of general formula (I) in which R⁴ represents C₁-C₃-alkyl or halogen.

In a preferred embodiment, the present invention covers compounds of general formula (I) in which R⁴ represents methyl or fluoro.

In a preferred embodiment, the present invention covers compounds of general formula (I) in which R⁴ represents methyl.

In a preferred embodiment, the present invention covers compounds of general formula (I) in which R⁴ represents fluoro.

In a preferred embodiment, the present invention covers compounds of general formula (I) in which R⁶ represents C₁-C₄-alkyl or halogen.

In a preferred embodiment, the present invention covers compounds of general formula (I) in which R⁶ represents C₁-C₃-alkyl or halogen.

In a preferred embodiment, the present invention covers compounds of general formula (I) in which R⁶ represents methyl or fluoro.

In a preferred embodiment, the present invention covers compounds of general formula (I) in which R⁶ represents methyl.

In a preferred embodiment, the present invention covers compounds of general formula (I) in which R⁶ represents fluoro.

In a preferred embodiment, the present invention covers compounds of general formula (I) in which R^{5a} represents hydrogen, phenyl or heteroaryl;

wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy, and wherein one of R^{5a} and R^{5b} represents hydrogen and the other represents phenyl or heteroaryl.

In a preferred embodiment, the present invention covers compounds of general formula (I) in which R^{5a} represents hydrogen or phenyl;

wherein said phenyl group is optionally substituted, one or more times, independently from each other, with halogen, C₁-C₄-alkyl, C₁-C₄-alkoxy, C₁-C₄-haloalkyl or C₁-C₄-haloalkoxy, and wherein one of R^{5a} and R^{5b} represents hydrogen and the other represents phenyl.

In a preferred embodiment, the present invention covers compounds of general formula (I) in which R^{5a} represents hydrogen or phenyl;

wherein said phenyl group is optionally substituted with methyl and wherein one of R^{5a} and R^{5b} represents hydrogen and the other represents phenyl.

In a preferred embodiment, the present invention covers compounds of general formula (I) in which R^{5b} represents hydrogen, phenyl or heteroaryl;

wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy and wherein one of R^{5a} and R^{5b} represents hydrogen and the other represents phenyl or heteroaryl.

In a further embodiment, the present invention covers compounds of general formula (I) in which R^{5b} represents hydrogen or phenyl;

wherein said phenyl group is optionally substituted, one or more times, independently from each other, with halogen, C₁-C₄-alkyl, C₁-C₄-alkoxy, C₁-C₄-haloalkyl or C₁-C₄-haloalkoxy and wherein one of R^{5a} and R^{5b} represents hydrogen and the other represents phenyl.

In a further embodiment, the present invention covers compounds of general formula (I) in which R^{5b} represents hydrogen or phenyl;

wherein said phenyl group is optionally substituted, one or two times, independently from each other, with fluoro, chloro, methyl, trifluoromethyl or trifluoromethoxy, and wherein one of R^{5a} and R^{5b} represents hydrogen and the other represents phenyl.

In a further embodiment, the present invention covers compounds of general formula (I) in which R^{5a} represents phenyl or heteroaryl;

wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy;
R^{5b} represents hydrogen.

In a further embodiment, the present invention covers compounds of general formula (I) in which R^{5a} represents phenyl;

wherein said phenyl group is optionally substituted, one or more times, independently from each other, with halogen, C₁-C₄-alkyl, C₁-C₄-alkoxy, C₁-C₄-haloalkyl or C₁-C₄-haloalkoxy;
R^{5b} represents hydrogen.

In a further embodiment, the present invention covers compounds of general formula (I) in which R^{5a} represents phenyl;

wherein said phenyl group is optionally substituted with methyl;
R^{5b} represents hydrogen.

In a further embodiment, the present invention covers compounds of general formula (I) in which R^{5a} represents hydrogen;
R^{5b} represents phenyl or heteroaryl;
   wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy.

In a further embodiment, the present invention covers compounds of general formula (I) in which R^{5a} represents hydrogen;
R^{5b} represents phenyl,
   wherein said phenyl group is optionally substituted, one or two times, independently from each other, with fluoro, chloro, methyl, trifluoromethyl or trifluoromethoxy.

In a further embodiment, the present invention covers compounds of general formula (I) in which n represents 0, 1 or 2.

In a further embodiment, the present invention covers compounds of general formula (I) in which n represents 0 or 1.

In a further embodiment, the present invention covers compounds of general formula (I) in which n represents 0.

It is to be understood that the present invention relates also to any combination of the preferred embodiments described above.

Preferred compounds are, namely
ethyl 4-({[1-(3-chlorophenyl)cyclopropyl]carbonyl}amino)-2-(1H-tetrazol-5-yl)benzoate;
cyclopentyl 4-({[1-(3-chlorophenyl)cyclopropyl]carbonyl}amino)-2-(1H-tetrazol-5-yl)benzoate;
sec-butyl 4-({[1-(3-chlorophenyl)cyclopropyl]carbonyl}amino)-2-(1H-tetrazol-5-yl)benzoate, as a mixture of enantiomers;
cyclopentyl 4-({[1-(3,4-difluorophenyl)cyclopropyl]carbonyl}amino)-2-(1H-tetrazol-5-yl)benzoate;
cyclopentyl 4-({[1-(2-fluoro-4-methylphenyl)cyclopropyl]carbonyl}amino)-2-(1H-tetrazol-5-yl)benzoate;
cyclopentyl 4-[({1-[2-fluoro-4-(trifluoromethyl)phenyl]cyclopropyl}carbonyl)amino]-2-(1H-tetrazol-5-yl)benzoate;
cyclopentyl 4-[({1-[2-fluoro-4-(trifluoromethoxy)phenyl]cyclopropyl}carbonyl)amino]-2-(1H-tetrazol-5-yl)benzoate;
sec-butyl 4-({[1-(3,4-difluorophenyl)cyclopropyl]carbonyl}amino)-2-(1H-tetrazol-5-yl)benzoate, as a mixture of enantiomers;
sec-butyl 4-({[1-(2-fluoro-4-methylphenyl)cyclopropyl]carbonyl}amino)-2-(1H-tetrazol-5-yl)benzoate, as a mixture of enantiomers;
sec-butyl 4-[({1-[2-fluoro-4-(trifluoromethyl)phenyl]cyclopropyl}carbonyl)amino]-2-(1H-tetrazol-5-yl)benzoate, as a mixture of enantiomers;
sec-butyl 4-[({1-[2-fluoro-4-(trifluoromethoxy)phenyl]cyclopropyl}carbonyl)amino]-2-(1H-tetrazol-5-yl)benzoate, as a mixture of enantiomers;
N-(cyclohexylmethyl)-4-[({1-[2-fluoro-4-(trifluoromethyl)phenyl]cyclopropyl}carbonyl)amino]-N-methyl-2-(1H-tetrazol-5-yl)benzamide;
cyclopentyl-4-({[trans-2-(3-methylphenyl)cyclopropyl]carbonyl}amino)-2-(1H-tetrazol-5-yl)benzoate, as a mixture of enantiomers;
1-methylcyclopentyl 4-[({1-[2-fluoro-4-(trifluoromethyl)phenyl]cyclopropyl}carbonyl)amino]-2-(1H-tetrazol-5-yl)benzoate;
2-methylbutan-2-yl 4-[({1-[2-fluoro-4-(trifluoromethyl)phenyl]cyclopropyl}carbonyl)amino]-2-(1H-tetrazol-5-yl)benzoate;
cyclohexyl 4-[({1-[2-fluoro-4-(trifluoromethyl)phenyl]cyclopropyl}carbonyl)amino]-2-(1H-tetrazol-5-yl)benzoate;
2-methoxyethyl 4-[({1-[2-fluoro-4-(trifluoromethyl)phenyl]cyclopropyl}carbonyl)amino]-2-(1H-tetrazol-5-yl)benzoate;
tetrahydrofuran-3-yl 4-[({1-[2-fluoro-4-(trifluoromethyl)phenyl]cyclopropyl}carbonyl)amino]-2-(1H-tetrazol-5-yl)benzoate, as a mixture of enantiomers;
(3S)-tetrahydrofuran-3-yl 4-[({1-[2-fluoro-4-(trifluoromethyl)phenyl]cyclopropyl}carbonyl)amino]-2-(1H-tetrazol-5-yl)benzoate;
(3R)-tetrahydrofuran-3-yl 4-[({1-[2-fluoro-4-(trifluoromethyl)phenyl]cyclopropyl}carbonyl)amino]-2-(1H-tetrazol-5-yl)benzoate;
tetrahydro-2H-pyran-4-yl 4-[({1-[2-fluoro-4-(trifluoromethyl)phenyl]cyclopropyl}carbonyl)amino]-2-(1H-tetrazol-5-yl)benzoate;
tetrahydro-2H-pyran-4-ylmethyl 4-[({1-[2-fluoro-4-(trifluoromethyl)phenyl]cyclopropyl}carbonyl)amino]-2-(1H-tetrazol-5-yl)benzoate;
1-methylpiperidin-4-yl 4-[({1-[2-fluoro-4-(trifluoromethyl)phenyl]cyclopropyl}carbonyl)amino]-2-(1H-tetrazol-5-yl)benzoate;
(2R)-butan-2-yl 4-[({1-[2-fluoro-4-(trifluoromethyl)phenyl]cyclopropyl}carbonyl)amino]-2-(1H-tetrazol-5-yl)benzoate;
(2S)-butan-2-yl 4-[({1-[2-fluoro-4-(trifluoromethyl)phenyl]cyclopropyl}carbonyl)amino]-2-(1H-tetrazol-5-yl)benzoate;
(2R)-butan-2-yl 4-[({1-[2-fluoro-4-(trifluoromethoxy)phenyl]cyclopropyl}carbonyl)amino]-2-(1H-tetrazol-5-yl)benzoate;
(2S)-butan-2-yl 4-[({1-[2-fluoro-4-(trifluoromethoxy)phenyl]cyclopropyl}carbonyl)amino]-2-(1H-tetrazol-5-yl)benzoate;
isobutyl 4-[({1-[2-fluoro-4-(trifluoromethyl)phenyl]cyclopropyl}carbonyl)amino]-2-(1H-tetrazol-5-yl)benzoate;
2,2-dimethylpropyl 4-[({1-[2-fluoro-4-(trifluoromethyl)phenyl]cyclopropyl}carbonyl)amino]-2-(1H-tetrazol-5-yl)benzoate;
cyclobutyl 4-[({1-[2-fluoro-4-(trifluoromethyl)phenyl]cyclopropyl}carbonyl)amino]-2-(1H-tetrazol-5-yl)benzoate;
2-methoxypropyl 4-[({1-[2-fluoro-4-(trifluoromethyl)phenyl]cyclopropyl}carbonyl)amino]-2-(1H-tetrazol-5-yl)benzoate, as a mixture of enantiomers;
1-methoxypropan-2-yl 4-[({1-[2-fluoro-4-(trifluoromethyl)phenyl]cyclopropyl}carbonyl)amino]-2-(1H-tetrazol-5-yl)benzoate, as a mixture of enantiomers;
1-(3-chlorophenyl)-N-[4-(piperidin-1-ylcarbonyl)-3-(1H-tetrazol-5-yl)phenyl]cyclopropanecarboxamide;
1-[2-fluoro-4-(trifluoromethyl)phenyl]-N-[4-(piperidin-1-ylcarbonyl)-3-(1H-tetrazol-5-yl)phenyl]cyclopropanecarboxamide;
1-[2-fluoro-4-(trifluoromethyl)phenyl]-N-{4-[(3-methoxypiperidin-1-yl)carbonyl]-3-(1H-tetrazol-5-yl)phenyl}cyclopropanecarboxamide, as a mixture of enantiomers;
1-[2-fluoro-4-(trifluoromethyl)phenyl]-N-{4-[(4-methoxypiperidin-1-yl)carbonyl]-3-(1H-tetrazol-5-yl)phenyl}cyclopropanecarboxamide;
N-[(2R)-butan-2-yl]-4-[({1-[2-fluoro-4-(trifluoromethyl)phenyl]cyclopropyl}carbonyl)amino]-2-(1H-tetrazol-5-yl)benzamide;
N-[(2S)-butan-2-yl]-4-[({1-[2-fluoro-4-(trifluoromethyl)phenyl]cyclopropyl}carbonyl)amino]-2-(1H-tetrazol-5-yl)benzamide;
N-cyclopentyl-4-[({1-[2-fluoro-4-(trifluoromethyl)phenyl]cyclopropyl}carbonyl)amino]-2-(1H-tetrazol-5-yl)benzamide;
4-({[1-(3-chlorophenyl)cyclopropyl]carbonyl}amino)-N-cyclopentyl-N-methyl-2-(1H-tetrazol-5-yl)benzamide;
N-cyclopentyl-4-[({1-[2-fluoro-4-(trifluoromethyl)phenyl]cyclopropyl}carbonyl)amino]-N-methyl-2-(1H-tetrazol-5-yl)benzamide;
N-cyclohexyl-4-[({1-[2-fluoro-4-(trifluoromethyl)phenyl]cyclopropyl}carbonyl)amino]-2-(1H-tetrazol-5-yl)benzamide;
N-cyclohexyl-4-[({1-[2-fluoro-4-(trifluoromethyl)phenyl]cyclopropyl}carbonyl)amino]-N-methyl-2-(1H-tetrazol-5-yl)benzamide;
N-(cyclohexylmethyl)-4-[({1-[2-fluoro-4-(trifluoromethyl)phenyl]cyclopropyl}carbonyl)amino]-2-(1H-tetrazol-5-yl)benzamide;
4-[({1-[2-fluoro-4-(trifluoromethyl)phenyl]cyclopropyl}carbonyl)amino]-N-(tetrahydro-2H-pyran-4-yl)-2-(1H-tetrazol-5-yl)benzamide;
4-({[1-(3-chlorophenyl)cyclopropyl]carbonyl}amino)-N-methyl-N-(tetrahydro-2H-pyran-4-yl)-2-(1H-tetrazol-5-yl)benzamide;
4-[({1-[2-fluoro-4-(trifluoromethyl)phenyl]cyclopropyl}carbonyl)amino]-N-methyl-N-(tetrahydro-2H-pyran-4-yl)-2-(1H-tetrazol-5-yl)benzamide;
4-[({1-[2-fluoro-4-(trifluoromethyl)phenyl]cyclopropyl}carbonyl)amino]-N-(tetrahydro-2H-pyran-4-ylmethyl)-2-(1H-tetrazol-5-yl)benzamide;
4-[({1-[2-fluoro-4-(trifluoromethyl)phenyl]cyclopropyl}carbonyl)amino]-N-methyl-N-(tetrahydro-2H-pyran-4-ylmethyl)-2-(1H-tetrazol-5-yl)benzamide;
4-[({1-[2-fluoro-4-(trifluoromethyl)phenyl]cyclopropyl}carbonyl)amino]-N-[(2R)-1-methoxypropan-2-yl]-2-(1H-tetrazol-5-yl)benzamide;
4-[({1-[2-fluoro-4-(trifluoromethyl)phenyl]cyclopropyl}carbonyl)amino]-N-[(2S)-1-methoxypropan-2-yl]-2-(1H-tetrazol-5-yl)benzamide;
4-[({1-[2-fluoro-4-(trifluoromethyl)phenyl]cyclopropyl}carbonyl)amino]-N-(2-methoxy-2-methylpropyl)-2-(1H-tetrazol-5-yl)benzamide;
4-[({1-[2-fluoro-4-(trifluoromethyl)phenyl]cyclopropyl}carbonyl)amino]-N-[(3R)-tetrahydrofuran-3-yl]-2-(1H-tetrazol-5-yl)benzamide;
4-[({1-[2-fluoro-4-(trifluoromethyl)phenyl]cyclopropyl}carbonyl)amino]-N-[(3S)-tetrahydrofuran-3-yl]-2-(1H-tetrazol-5-yl)benzamide;
4-[({1-[2-fluoro-4-(trifluoromethyl)phenyl]cyclopropyl}carbonyl)amino]-N-isobutyl-N-methyl-2-(1H-tetrazol-5-yl)benzamide;
4-[({1-[2-fluoro-4-(trifluoromethyl)phenyl]cyclopropyl}carbonyl)amino]-N-(5-methylpyridin-2-yl)-2-(1H-tetrazol-5-yl)benzamide;
4-[({1-[2-fluoro-4-(trifluoromethyl)phenyl]cyclopropyl}carbonyl)amino]-N-(6-methylpyridin-3-yl)-2-(1H-tetrazol-5-yl)benzamide;
N-sec-butyl-4-[({-[2-fluoro-4-(trifluoromethyl)phenyl]cyclopropyl}carbonyl)amino]-N-methyl-2-(1H-tetrazol-5-yl)benzamide, as single enantiomer 1;
ethyl 2-fluoro-4-[({1-[2-fluoro-4-(trifluoromethyl)phenyl]cyclopropyl}carbonyl)amino]-6-(1H-tetrazol-5-yl)benzoate;
ethyl 4-[({1-[2-fluoro-4-(trifluoromethyl)phenyl]cyclopropyl}carbonyl)amino]-2-methyl-6-(1H-tetrazol-5-yl)benzoate;
cyclopentyl 2-fluoro-4-[({-[2-fluoro-4-(trifluoromethyl)phenyl]cyclopropyl}carbonyl)amino]-6-(1H-tetrazol-5-yl)benzoate;
(3S)-tetrahydrofuran-3-yl 2-fluoro-4-[({1-[2-fluoro-4-(trifluoromethyl)phenyl]cyclopropyl}carbonyl)amino]-6-(1H-tetrazol-5-yl)benzoate;
2-fluoro-4-[({-[2-fluoro-4-(trifluoromethyl)phenyl]cyclopropyl}carbonyl)amino]-N-(5-methylpyridin-2-yl)-6-(1H-tetrazol-5-yl)benzamide;
cyclopentyl 4-[({1-[2-fluoro-4-(trifluoromethyl)phenyl]cyclopropyl}carbonyl)amino]-2-methyl-6-(1H-tetrazol-5-yl)benzoate;
(3S)-tetrahydrofuran-3-yl 4-[({1-[2-fluoro-4-(trifluoromethyl)phenyl]cyclopropyl}carbonyl)amino]-2-methyl-6-(1H-tetrazol-5-yl)benzoate; and
4-[({1-[2-fluoro-4-(trifluoromethyl)phenyl]cyclopropyl}carbonyl)amino]-2-methyl-N-(5-methylpyridin-2-yl)-6-(1H-tetrazol-5-yl)benzamide;
   and stereoisomers, tautomers, N-oxides, hydrates, solvates, and salts thereof, and mixtures of same.

As mentioned above, compounds of the present invention effectively inhibit Bradykinin B1 receptor and may therefore be used for the treatment or prophylaxis of diseases that are related to pain and to inflammation.

### Pharmaceutical Compositions of the Compounds of the Invention

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprises at least one compound according to the invention, conventionally together with one or more pharmaceutically suitable excipient(s), and to their use according to the present invention.

### Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations.

For example, the compounds of this invention can be combined with known hormonal therapeutical agents.

In particular, the compounds of the present invention can be administered in combination or as comedication with hormonal contraceptives. Hormonal contraceptives are for example Combined Oral Contraceptives (COCs) or Progestin-Only-Pills (POPs) or hormone-containing devices.

COCs include but are not limited to birth control pills or a birth control method that includes a combination of an estrogen (estradiol) and a progestogen (progestin). The estrogenic part is in most of the COCs ethinyl estradiol. Some COCs contain estradiol or estradiol valerate.

Said COCs contain the progestins norethynodrel, norethindrone, norethindrone acetate, ethynodiol acetate, norgestrel, levonorgestrel, norgestimate, desogestrel, gestodene, drospirenone, dienogest, or nomegestrol acetate.

Birth control pills include for example but are not limited to Yasmin, Yaz, both containing ethinyl estradiol and drospirenone; Microgynon or Miranova containing levonorgestrel and ethinyl estradiol; Marvelon containing ethinyl estradiol and desogestrel; Valette containing ethinyl estradiol and dienogest; Belara and Enriqa containing ethinyl estradiol and chlormadinonacetate; Qlaira containing estradiol valerate and dienogest as active ingredients; and Zoely containing estradiol and normegestrol.

POPs are contraceptive pills that contain only synthetic progestogens (progestins) and do not contain estrogen. They are colloquially known as mini pills.
POPs include but are not limited to Cerazette containing desogestrel; and Micronor containing norethindrone.

Other Progeston-Only forms are intrauterine devices (IUDs), for example Mirena containing levonorgestrel or injectables, for example Depo-Provera containing medroxyprogesterone acetate, or implants, for example Implanon containing etonogestrel.

Other hormone-containing devices with contraceptive effect which are suitable for a combination with the compounds of the present invention are vaginal rings like Nuvaring containing ethinyl estradiol and etonogestrel, or transdermal systems like contraceptive patches, for example Ortho-Evra containing ethinyl estradiol and norelgestromin or Apleek (Lisvy) containing ethinyl estradiol and gestodene.

A preferred embodiment of the present invention is the administration of a compound of general formula (I) in combination with a COC or a POP or other Progestin-Only forms, as well as in combination with vaginal rings or contraceptive patches as mentioned above.

Furthermore, the compounds of the present invention can be combined with therapeutic agents or active ingredients, that are already approved or that are still under development for the treatment and/or prophylaxis of diseases which are related to or mediated by the Bradykinin B1 receptor.

For the treatment and/or prophylaxis of urinary tract diseases, the compounds of the present invention can be administered in combination or as co-medication with any substance that can be applied as therapeutic agent in the following indications:

Urinary tract disease states associated with the bladder outlet obstruction; urinary incontinence conditions such as reduced bladder capacity, increased frequency of micturition, urge incontinence, stress incontinence, or bladder hyperreactivity; benign prostatic hypertrophy; prostatic hyperplasia; prostatitis; detrusor hyperreflexia; overactive bladder and symptoms related to overactive bladder wherein said symptoms are in particular increased urinary frequency, nocturia, urinary urgency or urge incontinence; pelvic hypersensitivity; urethritis; prostatitis; prostatodynia; cystitis, in particular interstitial cystitis; idiopathic bladder hypersensitivity.

For the treatment and/or prophylaxis of overactive bladder and symptoms related to overactive bladder, the compounds of the present invention can be administered in combination or as co-medication in addition to behavioural therapy like diet, lifestyle or bladder training with anticholinergics like oxybutynin, tolterodine, propiverine, solifenacin, darifenacin, trospium, fesoterdine; β-3 agonists like mirabegron; neurotoxins like onabutolinumtoxin A; or antidepressants like imipramine, duloxetine.

For the treatment and/or prophylaxis of interstitial cystitis, the compounds of the present invention can be administered in combination or as co-medication in addition to behavioural therapy like diet, lifestyle or bladder training with pentosans like elmiron; antidepressants like amitriptyline, imipramine; or antihistamines like loratadine.

For the treatment and/or prophylaxis of gynaecological diseases, the compounds of the present invention can be administered in combination or as co-medication with any substance that can be applied as therapeutic agent in the following indications:

dysmenorrhea, including primary and secondary; dyspareunia; endometriosis; endometriosis-associated pain; endometriosis-associated symptoms, such as and in particular dysmenorrhea, dyspareunia, dysuria, or dyschezia.

For the treatment and/or prophylaxis of dysmenorrhea, including primary and secondary; dyspareunia; endometriosis and endometriosis-associated pain, the compounds of the present invention can be administered in in combination with ovulation inhibiting treatment, in particular COCs as mentioned above or contraceptive patches like Ortho-Evra or Apleek (Lisvy); or with progestogenes like dienogest (Visanne); or with GnRH analogous, in particular GnRH agonists and antagonists, for example leuprorelin, nafarelin, goserelin, cetrorelix, abarelix, ganirelix, degarelix; or with androgens: danazol.

For the treatment and/or prophylaxis of diseases, which are associated with pain, or pain syndromes, the compounds of the present invention can be administered in combination or as co-medication with any substance that can be applied as therapeutic agent in the following indications:

pain-associated diseases or disorders like hyperalgesia, allodynia, functional bowel disorders (such as irritable bowel syndrome) and arthritis (such as osteoarthritis, rheumatoid arthritis and ankylosing spondylitis), burning mouth syndrome, burns, migraine or cluster headache, nerve injury, traumatic nerve injury, post-traumatic injuries (including fractures and sport injuries), neuritis, neuralgia, poisoning, ischemic injury, interstitial cystitis, viral, trigeminal neuralgia, small fiber neuropathy, diabetic neuropathy, chronic arthritis and related neuralgias, HIV and HIV treatment-induced neuropathy.

The compounds of the present invention can be combined with other pharmacological agents and compounds that are intended to treat inflammatory diseases, inflammatory pain or general pain conditions.

In addition to well-known medicaments which are already approved and on the market, the compounds of the present invention can be administered in combination with inhibitors of the P2X purinoceptor family (P2X3, P2X4), with inhibitors of IRAK4 and with antagonists of the prostanoid EP4 receptor.

In particular, the compounds of the present invention can be administered in combination with pharmacological endometriosis agents, intended to treat inflammatory diseases, inflammatory pain or general pain conditions and/or interfering with endometriotic proliferation and endometriosis associated symptoms, namely with inhibitors of Aldo-keto-reductase1C₃ (AKR1C₃) and with functional blocking antibodies of the prolactin receptor.

The compounds of the present invention can be combined with other pharmacological agents and compounds that are intended for the treatment, prevention or management of cancer.

In particular, the compounds of the present invention can be administered in combination with 131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, Iasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, or zorubicin.

Furthermore, the compounds of the present invention can be combined with active ingredients, which are well known for the treatment of cancer-related pain and chronic pain. Such combinations include, but are not limited to step II opiods like codeine phosphate, dextropropoxyphene, dihydro-codeine, Tramadol), step III opiods like morphine, fentanyl, buprenorphine, oxymorphone, oxycodone and hydromorphone; and other medications used for the treatment of cancer pain like steroids as Dexamethasone and methylprednisolone; bisphosphonates like Etidronate, Clodronate, Alendronate, Risedronate, and Zoledronate; tricyclic antidepressants like Amitriptyline, Clomipramine, Desipramine, Imipramine and Doxepin; class I antiarrhythmics like mexiletine and lidocaine; anticonvulsants like carbamazepine, Gabapentin, oxcarbazepine, phenytoin, pregabalin, topiramate, alprazolam, diazepam, flurazepam, pentobarbital and phenobarbital.

In addition to those mentioned above, the inventive Bradykinin B1 inhibitors can also be combined with any of the following active ingredients:

active ingredients for Alzheimer's therapy, for example acetylcholinesterase inhibitors (e.g. donepezil, rivastigmine, galantamine, tacrine), NMDA (N-methyl-D-aspartate) receptor antagonists (e.g. memantine); L-DOPA/carbidopa (L-3,4-dihydroxyphenylalanine), COMT (catechol-O-methyltransferase) inhibitors (e.g. entacapone), dopamine agonists (e.g. ropinrole, pramipexole, bromocriptine), MAO-B (monoaminooxidase-B) inhibitors (e.g. selegiline), anticholinergics (e.g. trihexyphenidyl) and NMDA antagonists (e.g. amantadine) for treatment of Parkinson's; beta-interferon (IFN-beta) (e.g. IFN beta-1b, IFN beta-1a Avonex® and Betaferon®), glatiramer acetate, immunoglobulins, natalizumab, fingolimod and immunosuppressants such as mitoxantrone, azathioprine and cyclophosphamide for treatment of multiple sclerosis; substances for treatment of pulmonary disorders, for example beta-2-sympathomimetics (e.g. salbutamol), anticholinergics (e.g. glycopyrronium), methylxanthines (e.g. theophylline), leukotriene receptor antagonists (e.g. montelukast), PDE-4 (phosphodiesterase type 4) inhibitors (e.g. roflumilast), methotrexate, IgE antibodies, azathioprine and cyclophosphamide, cortisol-containing preparations; substances for treatment of osteoarthritis such as non-steroidal anti-inflammatory substances (NSAIDs). In addition to the two therapies mentioned, methotrexate and biologics for B-cell and T-cell therapy (e.g. rituximab, abatacept) should be mentioned for rheumatoid disorders such as rheumatoid arthritis and juvenile idiopathic arthritis. Neurotrophic substances such as acetylcholinesterase inhibitors (e.g. donepezil), MAO (monoaminooxidase) inhibitors (e.g. selegiline), interferons and anticonvulsives (e.g. gabapentin); active ingredients for treatment of cardiovascular disorders such as beta-blockers (e.g. metoprolol), ACE inhibitors (e.g. benazepril), diuretics (e.g. hydrochlorothiazide), calcium channel blockers (e.g. nifedipine), statins (e.g. simvastatin); anti-diabetic drugs, for example metformin and glibenclamide, sulphonylureas (e.g. tolbutamide) and insulin therapy for treatment of diabetes and metabolic syndrome. Active ingredients such as mesalazine, sulfasalazine, azathioprine, 6-mercaptopurine or methotrexate, probiotic bacteria (Mutaflor, VSL#3®, Lactobacillus GG, Lactobacillus plantarum, L. acidophilus, L. casei, Bifidobacterium infantis 35624, Enterococcus fecium SF68, Bifidobacterium longum, Escherichia coli Nissle 1917), antibiotics, for example ciprofloxacin and metronidazole, anti-diarrhoea drugs, for example loperamide, or laxatives (bisacodyl) for treatment of chronic-inflammatory bowel disorders. Immunosuppressants such as glucocorticoids and non-steroidale anti-inflammatory substances (NSAIDs), cortisone, chloroquine, cyclosporine, azathioprine, belimumab, rituximab, cyclophosphamide for treatment of lupus erythematosus. By way of example but not exclusively, calcineurin inhibitors (e.g. tacrolimus and ciclosporin), cell division inhibitors (e.g. azathioprine, mycophenolate mofetil, mycophenolic acid, everolimus or sirolimus), rapamycin, basiliximab, daclizumab, anti-CD3 antibodies, anti-T-lymphocyte globulin/anti-lymphocyte globulin for organ transplants, Vitamin D3 analogues, for example calcipotriol, tacalcitol or calcitriol, salicylic acid, urea, ciclosporine, methotrexate, or efalizumab for dermatological disorders.

### Methods of Treating

The present invention relates to a method for using the compounds of the present invention and compositions thereof, to inhibit the Bradykinin B1 receptor.

The present invention relates to a method for using the compounds of the present invention and compositions thereof, to treat mammalian disorders and diseases which include but are not limited to:

Diseases related to pain and inflammation, in particular selected from the group consisting of
visceral pain e.g. related to pancreatitis, interstitial cystitis, renal colic, or prostatitis, chronic pelvic pain, or pain related to infiltrating endometriosis;
neuropathic pain such as post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes potentially caused by virtually any lesion at any level of the nervous system including but not limited to pain related to stroke, multiple sclerosis, and spinal cord injury;
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative, and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis);

and diseases like or related to a disease selected from related to the group consisting of:
gynaecological disorders and/or diseases, or effects and/or symptoms which negatively influence women health including endometriosis, uterine fibroids, pre-eclampsia, hormonal deficiency, spasms of the uterus, or heavy menstrual bleeding;
the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis;
diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, or fat or muscle metabolism;
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including primary peritonitis, secondary peritonitis, septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.

A preferred embodiment of the present invention relates to a method for using the compounds of the present invention and compositions thereof, to treat a gynaecological disease, preferably dysmenorrhea, dyspareunia or endometriosis, endometriosis-associated pain, or other endometriosis-associated symptoms, wherein said symptoms include dysmenorrhea, dyspareunia, dysuria, or dyschezia.

Additionally the present invention relates to a method for using the compounds of the present invention and compositions thereof, to treat osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a gynaecological disease.

### Dose and Administration

Based upon standard laboratory techniques known to evaluate compounds useful for the treatment of disorders and/or diseases which are mediated by Bradykinin B1 receptor, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, preferably from about 0.01 mg/kg to about 20 mg/kg body weight per day. A preferred administration of the compound of the present invention includes but is not limited to 0.1 mg/kg to about 10 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. A preferred oral unit dosage for administration of the compounds of the present invention includes but is not limited to 0.1 mg/kg to about 10 mg/kg body weight one to three times a day to once a week. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg of total body weight. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases treated with said method are gynaecological disorders, more preferably dysmenorrhea, dyspareunia or endometriosis, endometriosis-associated pain, or other endometriosis-associated symptoms, wherein said symptoms include dysmenorrhea, dyspareunia, dysuria, or dyschezia. Further diseases which can be treated with said method are osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

Preferably, the method of treating the diseases mentioned above is not limited to the treatment of said disease but also includes the treatment of pain related to or associated with said diseases.

The compounds of the present invention can be used in particular in therapy and prevention, i.e. prophylaxis, of genitourinary, gastrointestinal, respiratory or pain-related disease, condition or disorder.

Methods of testing for a particular pharmacological or pharmaceutical property are well known to persons skilled in the art.

The example testing experiments described herein serve to illustrate the present invention and the invention is not limited to the examples given.

### Synthesis of Compounds of General Formula (I) of the Present Invention

Compounds of general formula (I) with the meaning of R¹, R², R³, R⁴, R^{5a}, R^{5b}, A, X and Z as defined in general formula (I), can be synthesised according to various general procedures.

Scheme 1 depicts the synthesis starting from synthons of the general formula (II), which can be ring opened in excess alcohol (for example ethanol) at temperatures between 20° C. and 80° C. to yield compounds of the general formula (III). The carboxylic acid moiety of general formula (III) can be converted to acid chlorides of general formula (IV) by treatment with thionyl chloride in the presence of catalytic N,N-dimethylformamide at temperatures between 20° C. and 70° C. Treatment of a compound of general formula (IV) with ammonia in a suitable solvent (for example dichloromethane or 1,4-dioxane, or mixtures thereof) provides carbamoyl compounds of general formula (V). These in turn can be converted to nitriles of general formula (VI) by treatment with excess thionyl chloride at elevated temperatures, between 60° C. and 100° C. The nitrile moiety of a compound of general formula (VI) is converted to tetrazoles of the general formula (VII) by reaction with 1-4 equivalents of trimethylsilyl azide in the presence of 1-2 equivalents of dibutyltinoxide in a suitable solvent (for example toluene or xylene) at temperatures between 50° C. and 160° C. The 1H-tetrazoles of the present invention has to be understood as both 1H- and 2H-tautomers. The nitro group of a compound of general formula (VII) is then reduced to the corresponding aromatic amine of general formula (VIII) by reaction under a hydrogen atmosphere in the presence of a palladium catalyst (for example 5-10% palladium on carbon) in an appropriate solvent (for example ethanol or ethyl acetate) at temperatures between 0° C. and 100° C. The order of the preceding two steps can also be reversed, with nitro reduction being carried out prior to the tetrazole formation. Aromatic amines of general formula (VIII) may react with a carboxylic acid of general formula (IX) by methods known to those skilled in the art to give compounds of the general formula (XI). The reaction is mediated by activating a carboxylic acid of general formula (IX) with reagents such as dicyclohexylcarbodiimide (DCC), 1-(3-dimethylaminopropyl)-3-ethylcarbodiimide (EDCI), N-hydroxybenzotriazole (HOBT), N-[(dimethylamino)-(3H-[1,2,3]triazolo[4,5-b]pyridin-3-yl oxy)methyliden]-N-methylmethanaminium hexafluorophosphate (HATU) or propylphosphonic anhydride (T3P). For example, the reaction with HATU takes place in an inert solvent, such as N,N-dimethylformamide, dichloromethane or dimethyl sulfoxide in the presence of the appropriate aromatic amine of general formula (VIII) and a tertiary amine (such as triethylamine or diisopropylethylamine) at temperatures between −30° C. and +90° C.

It is also possible to convert a carboxylic acid of the general formula (IX) into the corresponding carboxylic acid chloride (X) with an inorganic acid chloride (such as phosphorus pentachloride, phosphorus trichloride or thionyl chloride) and then into the target compounds of the general formula (XI), in pyridine or an inert solvent (such as N,N-dimethylformamide), in the presence of the appropriate aromatic amine formula (VIII) and a tertiary amine (for example triethylamine) at temperatures between −30° C. and +90° C.

For compounds of general formula (I) with Z being OR¹ and R¹ being ethyl, the steps following the compound of general formula (XI) in scheme 1 may be omitted.

Alkyl esters of the general formula (XI) can be hydrolysed with an aqueous base (for example sodium hydroxide or lithium hydroxide) in an appropriate solvent (for example tetrahydrofuran, methanol, water or mixtures thereof) to give carboxylic acids of general formula (XII). Treatment of carboxylic acids of the general formula (XII) with an excess of the appropriate alcohol in the presence of catalytic sulfuric acid at temperatures between 20° C. and 80° C. gives target compounds of the general formula (I).

Furthermore, it is possible to convert a carboxylic acid of general formula (XII) into the corresponding secondary or tertiary amide of general formula (I). The carboxylic acid of general formula (XII) is first activated with a reagent such as HATU (or alternatively DCC, EDCI, HOBT or T3P) in an inert solvent (such as N,N-dimethylformamide or dichloromethane) in the presence of the appropriate primary or secondary amine (Z—H) and a tertiary amine (such as diisopropylethylamine) at temperatures between −30° C. and +90° C. to give target compounds of general formula (I).

The carboxylic acids of general formula (IX) are either commercially available or can be synthesised via methods known to those skilled in the art from appropriate precursors. For example, arylcyclopropanecarboxylic acids may be prepared from the corresponding arylacetonitrile by cyclopropanation with 1-bromo-2-chloroethane (1.5 eq) in aqueous sodium hydroxide solution in the presence of 0.02 eq. benzyltriethylammonium chloride and subsequent acidic or basic hydrolysis of the nitrile with e.g. lithium hydroxide in water or concentrated hydrochloric acid at temperatures between 20° C. and 100° C.

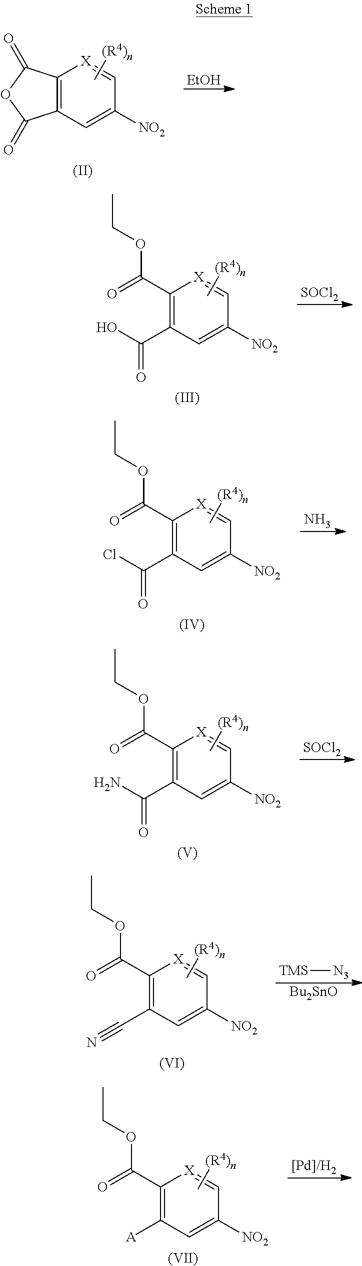

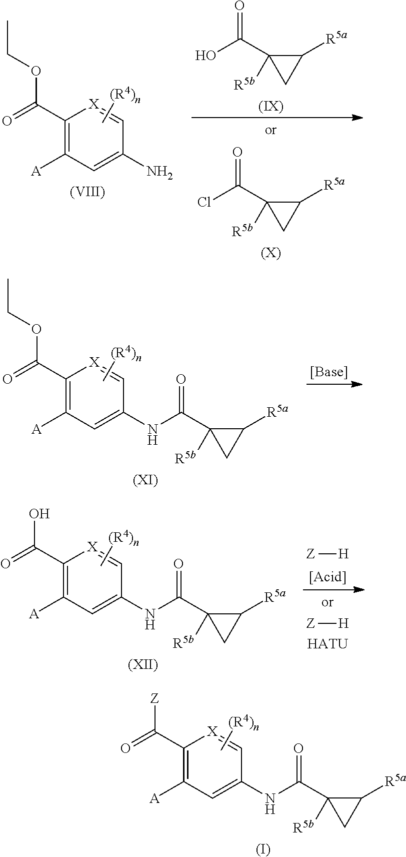

In Scheme 1, Z—H represents R¹O—H in combination with [Acid]. Alternatively, Z—H represents H—NR²R³ in combination with a carboxylic acid activating reagent such as HATU.

An alternative approach to access intermediates of the general formula (VI) is shown in Scheme 2. Synthons of the general formula (XIII) (wherein Hal stands for Cl, Br or I; Cl being preferred) are esterified using excess of ethanol in the presence of catalytic sulfuric acid at temperatures between 80° C. and 120° C. The resulting halides of general formula (XIV) can then be reacted with copper cyanide to yield a compound of general formula (VI). A suitable solvent (for example N, N-dimethylformamide or N-methyl-2-pyrrolidone) is used and a catalyst-ligand mixture, for example of palladium(II) acetate/triphenylphosphine, tetrakis(triphenylphosphine)palladium(0), tris(dibenzylideneacetone)dipalladium, bis(triphenylphosphine)palladium(II) dichloride, bis(diphenylphosphino)ferrocenedichloropalladium (II) is utilised at temperatures between 90° C. and 150° C.

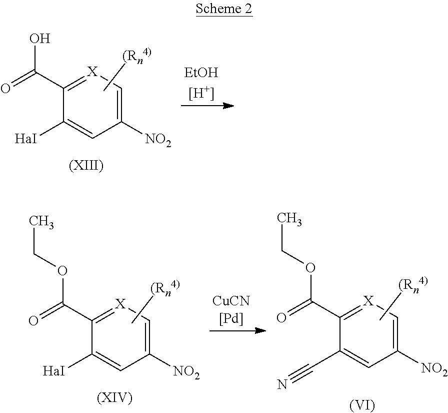

Intermediates of the general formula (XI) can also be accessed via the route shown in Scheme 3. Synthons of the general formula (XV) (wherein Hal stands for Cl, Br or I; Br being preferred) undergo amide coupling in analogy to the procedures shown in Scheme 1 giving compounds of the general formula (XVI). The aryl halide can then be reacted with ethanol in the presence of carbon monoxide by Pd-mediatated reaction to give ethyl ester intermediates of the general formula (XVII). A catalyst-ligand mixture, for example of palladium (II) acetate/triphenylphosphine, tetrakis(triphenylphosphine)palladium(0), tris(dibenzylideneacetone)dipalladium, bis(triphenylphosphine)palladium(II) dichloride or bis(diphenylphosphino)ferrocenedichloropalladium (II) and a base (such as triethylamine, potassium carbonate or potassium acetate) is utilised at temperatures between 100° C. and 180° C., using carbon monoxide at pressures of between 1 to 40 bar. Subsequent tetrazole formation yields the intermediate of general formula (XI). In analogy to the procedures described for Scheme 1, ester group hydrolysis gives compounds of the general formula (XII), followed by either esterification in catalytic acid or amidation with a primary or secondary amine provides access to the target compounds of the general formula (I).

For compounds of general formula (I) with Z being OR¹ and R¹ being ethyl, the steps following the compound of general formula (XI) in scheme 1 may be omitted.

The Synthons of the general formula (XV) can be synthesised via methods known to those skilled in the art from appropriate precursors. For example, 2-halo-5-aminobenzonitriles can be obtained by reduction of the corresponding nitro compounds with iron powder in ethanol and acetic acid at temperatures between rt and 100° C. The respective nitro compounds can be obtained e.g. by nitration of the corresponding 2-halo-benzonitriles with sulfuric acid/nitric acid (69%) in a v/v 4:1 ratio between 0° C. and RT.

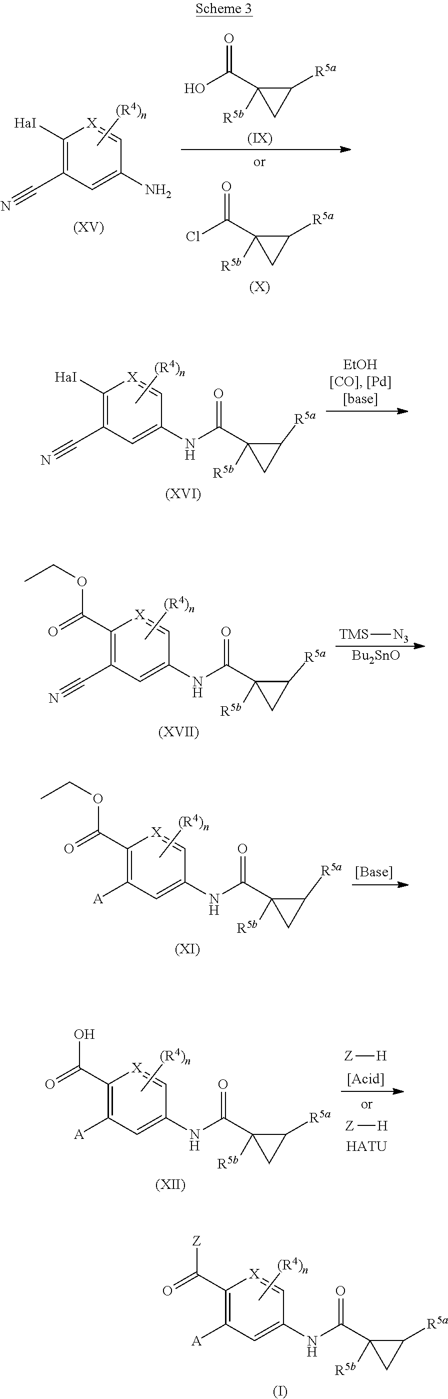

Scheme 4 shows an alternative approach to synthesise compounds of the general formula (I). Synthons of the general formula (XVIII) can be Boc-protected by methods known to those skilled in the art using 1-1.5 equivalents di-tert-butyl dicarbonate and a suitable base (for example trimethylamine or sodium carbonate) in an appropriate solvent (for example acetonitrile, ethyl acetate or water) to provide compounds of the general formula (XIX).

The resulting compounds can be ring opened to afford the desired esters of general formula (XX) by reaction in the appropriate alcohol at elevated temperature between 70° C. and 120° C. Alternatively, compounds of the general formula (XIX) can be ring opened with the appropriate primary or secondary amine at a temperature between room temperature and 120° C. to afford the desired secondary or tertiary amide of general formula (XX). Subsequent Boc group deprotection to compounds of general formula (XXI) can be carried out using hydrochloric acid or trifluoroacetic acid in a suitable solvent (for example dichloromethane or 1,4-dioxane). In analogy to the procedures described for Scheme 1, treatment with thionyl chloride gives compounds of the general formula (XXII), followed by tetrazole formation which yields nitro compounds of the general formula (XXIII). Nitro group reduction followed by amide formation with compounds of general formula (IX) or general formula (X) provides access to the target compounds of general formula (I).

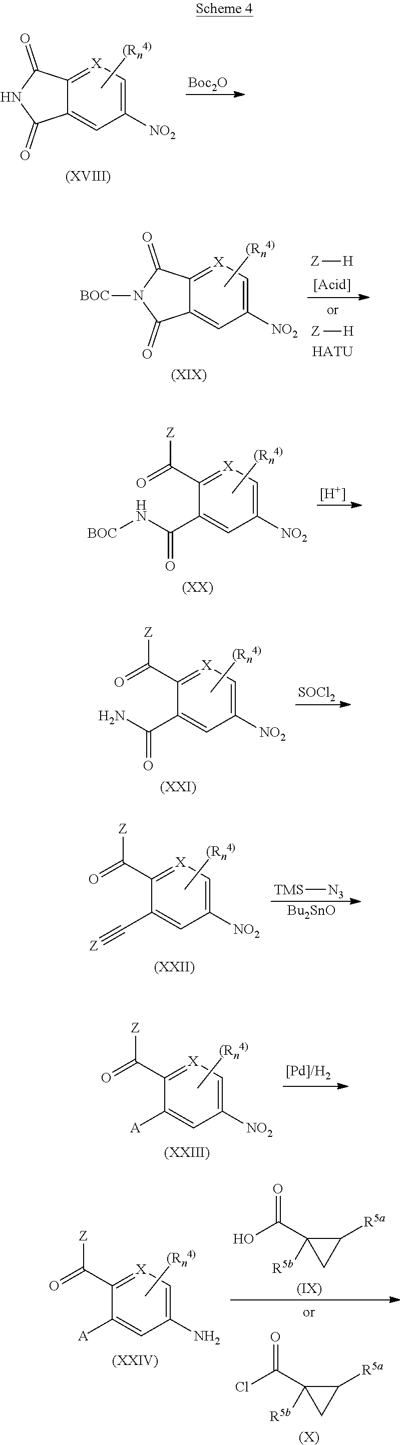

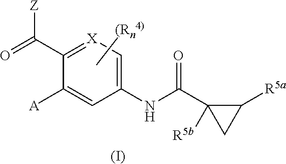

Wherein Z—H represents R¹—OH in combination with [Acid]. Alternatively, Z—H represents H—NR²R³ in combination with a carboxylic acid activating reagent such as HATU.

Scheme 5 shows an alternative approach to synthesise compounds of general formula (I). In analogy to the procedure conditions described for Scheme 1, ester group hydrolysis of a compound of general formula (XXV) with an aqueous base (for example lithium hydroxide) in an appropriate solvent (for example tetrahydrofuran) provides carboxylic acid compounds of general formula (XXVI). The resulting compounds can either be esterified with an excess of the appropriate alcohol (Z—H) in catalytic sulfuric acid or alternatively amidated with the appropriate primary or secondary amine (Z—H) using a carboxylic acid activating reagent (such as HATU or T3P) and a tertiary base (such as diisopropylethylamine) to give compounds of general formula (XXVII). Nitro group reduction followed by amide formation with compounds of general formula (IX) or general formula (X) provides access to the target compounds of general formula (I).

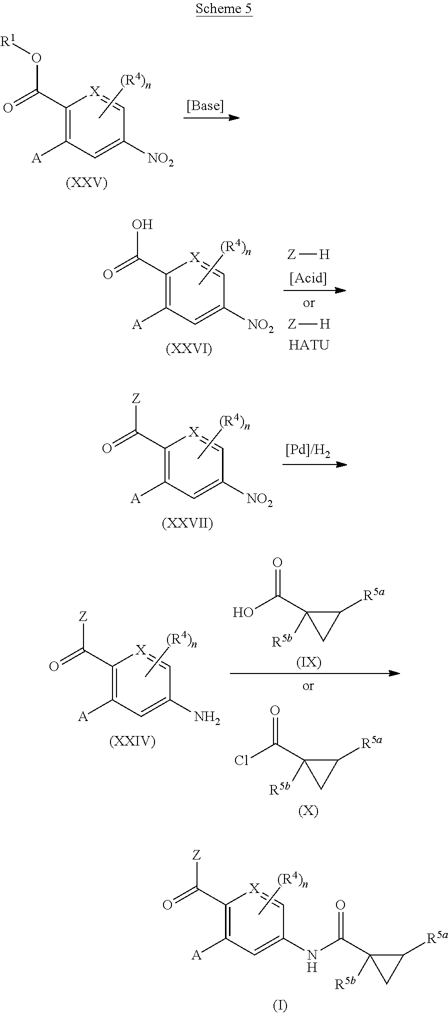

### EXPERIMENTAL SECTION

The example testing experiments described herein serve to illustrate the present invention and the invention is not limited to the examples given.

The following table lists the abbreviations used in this paragraph, and in the examples section.

| Abbreviation | Meaning |
|---|---|
| Cs₂CO₃ | Cesium carbonate |
| Cu(I)Cl | Copper(I) chloride |
| ca. | circa |
| DCE | 1,2-Dichloroethane |
| DCM | Dichloromethane |
| DIAD | Diisiopropyl azodicarboxylate |
| DIPEA | N-Ethyl-N-isopropylpropan-2-amine |
| DIAD | Diisopropyl azodicarboxylate |
| DMA | Dimethylacetamide |
| DMAP | N,N-Dimethylpyridin-4-amine |
| DMF | N,N-Dimethylformamide |
| DMSO | Dimethyl sulfoxide |
| DP | Desired product |
| EE | Ethyl acetate |
| EEDQ | N-Ethoxycarbonyl-2-ethoxy-1,2-dihydroquinoline |
| h | Hour |
| HATU | N-[(Dimethylamino)(3H-[1,2,3]triazolo[4,5- |
| | b]pyridin-3-yloxy)methylene]-N- |
| | methylmethanaminium hexafluorophosphate |
| HBr | Hydrogen bromide |
| HCl | Hydrochloric acid |
| HPLC | high performance liquid chromatography |
| IPC | In process check |
| K₂CO₃ | Potassium carbonate |
| LC-MS | liquid chromatography - mass spectrometry |
| LiOH | Lithium hydroxide |
| M | Molar |
| μW | Microwave |
| MeCN | Acetonitile |
| MeOH | Methanol |
| MgSO₄ | Magnesium sulfate |
| min | Minute(s) |
| N | Normal |
| Na₂CO₃ | Sodium carbonate |
| NaH | Sodium hydride |
| NaHCO₃ | Sodium bicarbonate |
| NaI | Sodium iodide |
| NaOH | Sodium hydroxide |
| Na₂SO₄ | Sodium sulfate |
| NH₄Cl | Ammonium chloride |
| NMP | N-Methyl-2-pyrrolidinone |
| NMR | nuclear magnetic resonance spectroscopy |
| PdCl₂(PPh₃)₂ | Bis(triphenylphosphine)palladium(II) dichloride |
| Pd(dppf)Cl₂ | [1,1′-Bis(diphenylphosphino)ferrocene] |
| | dichloropalladium(II) |
| Pd(dppf)Cl₂•CH₂Cl₂ | 1,1′-Bis(diphenylphosphino)ferrocene- |
| | palladium(II)dichloride dichloromethane complex |
| PPh₃ | Triphenylphosphine |
| ppm | parts per million |
| RT | Room temperature |
| rt | Retention time |
| Rt | Retention time |
| sat. | Saturated |
| SEM | 2-(trimethylsilyl)ethoxymethyl |
| SM | Starting material |
| STAB | Sodium triacetoxyborohydride |
| T3P | Propylphosphonic anhydride |
| TBAI | Tetra-N-butylammonium iodide |
| TBME | tert-Butyl methyl ether |
| TEA | Triethylamine |
| TFA | Trifluoroacetic acid |
| THF | Tetrahydrofuran |

### Analysis Methods

### Analytical LCMS Methods

Method 1: Instrument: Waters Acquity Platform ZQ4000; column: Waters BEHC 18, 50 mm×2.1 mm, 1.7μ; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.
Method 2: Instrument: Waters Acquity LCT; column: Phenomenex Kinetex C18, 50 mm×2.1 mm, 2.6μ; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.
Method 3: Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.
Method 4: Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.
Method 5: Instrument MS: Waters ZQ; instrument HPLC: Waters UPLC Acquity; column: Acquity BEH C₁₈ (Waters), 50 mm×2.1 mm, 1.7 μm; eluent A: water+0.1% formic acid, eluent B: acetonitrile (Lichrosolv Merck); gradient: 0.0 min 99% A-1.6 min 1% A-1.8 min 1% A-1.81 min 99% A-2.0 min 99% A; oven: 60° C.; flow: 0.800 ml/min; UV-detection PDA 210-400 nm.
Method 6: Instrument: Waters Acquity UPLCMS Single Quad; column: Kinetex 2.6, 50×2.1 mm; Eluent A: water+0.05% formic acid (99%); Eluent B: acetonitrile+0.05% formic acid (99%); gradient: 0-1.9 1-99% B, 1.9-2.1 99% B; flow 1.3 ml/min; temperature: 60° C.; DAD scan: 200-400 nm.

### Analytical LCMS, Method 1, Low pH:

Instrument: Waters Acquity UPLCMS SingleQuad; Column: Acquity UPLC BEH C18 1.7 □50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

### Analytical LCMS, Method 2, High pH:

Instrument: Waters Acquity UPLCMS SingleQuad; Column: Acquity UPLC BEH C18 1.7 □50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

### LC-MS, Analytical Method A: Routine High Throughput Analysis

Column: Kinetex Core-Shell C18, 2.1×50 mm, 5 μm; Eluent A: Water+0.1% Formic acid, Eluent B: Acetonitrile+0.1% Formic acid; Gradient 0.00 mins 95% A→1.20 mins 100% B→1.30 mins 100% B→1.31 mins 95% A; column temperature: 40° C.; flow rate 1.2 ml/min; injection volume: 3 μl; UV-detection range: 210-420 nm.

### LC-MS, Analytical Method C: Routine High Throughput Analysis at High pH

Column: Phenomenex Gemini-NX C18, 2.0×50 mm, 3 μm; Eluent A: 2 mM ammonium bicarbonate, buffered to pH10, Eluent B: Acetonitrile; Gradient 0.00 mins 99% A→1.80 mins 100% B→2.10 mins 100% B→2.30 mins 99% A→3.50 mins 99% A; column temperature: 40° C.; flow rate 1.0 ml/min; injection volume: 3 μl; UV-detection range: 210-420 nm.

### LC-MS, Analytical Method D:

Column: Waters Atlantis dC18, 2.1×100 mm, 3 μm; Eluent A: Water+0.1% Formic acid, Eluent B: Acetonitrile+0.1% Formic acid; Gradient 0.00 mins 95% A→5.00 mins 100% B→5.40 mins 100% B→5.42 mins 95% A→7.00 mins 95% A; column temperature: 40° C.; flow rate 0.6 ml/min; injection volume: 3 μl; UV-detection range: 210-420 nm.

### LC-MS, Analytical Method E: High pH

Column: Phenomenex Gemini-NX C18, 2.0×100 mm, 3 μm; Eluent A: 2 mM ammonium bicarbonate, buffered to pH10, Eluent B: Acetonitrile; Gradient 0.00 mins 95% A→5.50 mins 100% B→5.90 mins 100% B→5.92 mins 95% A→7.00 mins 95% A; column temperature: 40° C.; flow rate 0.5 ml/min; injection volume: 3 μl; UV-detection range: 210-420 nm.

### LC-MS, Analytical Method F:

Column: Phenomenex Kinetix-XB C18, 2.1×100 mm, 1.7 μm; Eluent A: Water+0.1% Formic acid, Eluent B: Acetonitrile+0.1% Formic acid; Gradient 0.00 mins 95% A→5.30 mins 100% B→5.80 mins 100% B→5.82 mins 95% A→7.00 mins 95% A; column temperature: 40° C.; flow rate 0.6 ml/min; injection volume: 1 μl; UV-detection range: 200-400 nm.

### Purification Methods:

Biotage Isolera™ chromatography system using pre-packed silica and pre-packed modified silica cartridges.

### Preparative HPLC, Method A: High pH

Column: Waters Xbridge C18, 30×100 mm, 10 μm; Solvent A: Water+0.2% Ammonium hydroxide, Solvent B: Acetonitrile+0.2% Ammonium hydroxide; Gradient 0.00 mins 90% A→0.55 mins 90% A→14.44 mins 95% B→16.55 mins 95% B→16.75 90% A; column temperature: room temperature; flow rate 40 ml/min; injection volume: 1500 μA; Detection: UV 215 nm.

### Preparative HPLC, Method B: Low pH

Column: Waters Sunfire C18, 30×100 mm, 10 μm; Solvent A: Water+0.1% Formic acid, Solvent B: Acetonitrile+0.1% Formic acid; Gradient 0.00 mins 90% A→0.55 mins 90% A→14.44 mins 95% B→16.55 mins 95% B→16.75 90% A; column temperature: room temperature; flow rate 40 ml/min; injection volume: 1500 μA; Detection: UV 215 nm.

### Preparative HPLC Methods

### Preparative HPLC, Method 1:

System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. formic acid (99%), B=acetonitrile; Gradient: 0-8 min 10-100% B, 8-10 min 100% B; Flow: 50 mL/min; temperature: room temp.; Solution: Max. 250 mg/max. 2.5 mL DMSO o. DMF; Injection: 1×2.5 mL; Detection: DAD scan range 210-400 nm; MS ESI+, ESI−, scan range 160-1000 m/z.

### Preparative HPLC, Method 2:

System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. ammonia (99%), B=acetonitrile; Gradient: 0-8 min 10-100% B, 8-10 min 100% B; Flow: 50 mL/min; temperature: room temp.; Solution: Max. 250 mg/max. 2.5 mL DMSO o. DMF; Injection: 1×2.5 mL; Detection: DAD scan range 210-400 nm; MS ESI+, ESI−, scan range 160-1000 m/z.

### Chiral Purification Methods:

| | | | Detector | |
|---|---|---|---|---|
| | | Flow rate | wavelength | Isocratic |
| Method | Column Type | (mL/min) | (nm) | Conditions |
| 1 | Lux A1 (21.2 mm × | 15 | 220 | 85:15 Heptane/ |
| | 250 mm, 5□m) | | | EtOH (0.1% |
| | | | | v/v TFA) |

### Chiral Analysis Methods:

| | | | Detector | |
|---|---|---|---|---|
| | | Flow rate | wavelength | Isocratic |
| Method | Column Type | (mL/min) | (nm) | Conditions |
| 1 | Lux C3 (4.6 mm × | 1 | 254 | 85:15 Heptane/ |
| | 150 mm, 5□m) | | | EtOH (0.1% |
| | | | | v/v TFA) |

### EXAMPLES

Chemical naming of the Examples and Intermediates was performed using ACD software by ACD/LABS or Marvin software by ChemAxon.

Reaction times are either specified explicitly in the protocols of the experimental section, or reactions were run until completion. Chemical reactions were monitored and their completion was judged using methods well known to the person skilled in the art, such as thin layer chromatography, e.g. on plates coated with silica gel, or by LCMS methods.

### Intermediate 1A: 2-(Ethoxycarbonyl)-5-nitrobenzoic Acid

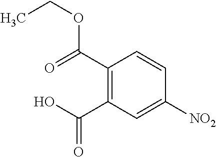 [SMILES: CCOC(=O)c1ccc([N+](=O)[O-])cc1C(=O)O]

5-Nitro-1,3-dihydro-2-benzofuran-1,3-dione (19.9 g, 103 mmol) was heated in EtOH (120 mL) at 70° C. for 1 hour giving a pale yellow solution. The mixture was concentrated at reduced pressure and TBME (100 mL) was added and the mixture was re-concentrated. This was repeated twice more and the resulting solid was dried. The solid residue was then dissolved in TBME (˜50 mL) at 70° C. and heptane (˜100 mL) was added slowly with heating until a cloudy solution formed. The mixture was then cooled to room temperature and the solids filtered and dried giving the product as a 7:3 mixture of isomers (11 g). This was repeated twice more to afford 4.7 g (18% yield) of the title compound as an off white solid as a 96:4 mixture of isomers.

¹H NMR (250 MHz, Chloroform-d) δ [ppm] 10.63 (s, 1H), 8.78 (s, 1H), 8.46 (d, J=8.4 Hz, 1H), 7.86 (d, J=8.4 Hz, 1H), 4.45 (q, J=7.1 Hz, 2H), 1.40 (t, J=7.1 Hz, 3H).

### Intermediate 2A: Ethyl 2-carbamoyl-4-nitrobenzoate

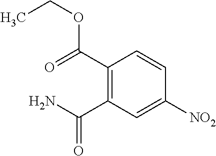 [SMILES: CCOC(=O)c1ccc([N+](=O)[O-])cc1C(N)=O]

2-(Ethoxycarbonyl)-5-nitrobenzoic acid (Int. 1A, 2.5 g, 10.3 mmol) was dissolved in thionyl chloride (15 mL) and N,N-Dimethylformamide (3 drops) was added. The resulting mixture was heated to 40° C. for 2 hours until no further gas evolution was observed. The reaction mixture was then concentrated at reduced pressure to remove excess thionyl chloride. The residue was then dissolved in dichloromethane (20 mL) and added dropwise to a 0.5 M solution of ammonia in 1,4-dioxane (44 mL, 22 mmol) at 0° C. The mixture was allowed to warm to room temperature and then concentrated at reduced pressure. The residue was diluted with ethyl acetate (100 mL) and washed with brine (3×40 mL), dried (Na₂SO₄), filtered and concentrated at reduced pressure. The residue was recrystallised with heptane/ethyl acetate to afford 1.12 g (47% yield) of the title compound as an off white solid.

¹H NMR (500 MHz, DMSO-d6) δ[ppm] 8.41-8.34 (m, 2H), 8.26 (s, 1H), 7.93-7.86 (m, 1H), 7.74 (s, 1H), 4.27 (q, J=7.1 Hz, 2H), 1.28 (t, J=7.1 Hz, 3H).

LCMS (Analytical Method A): Rt=0.83 mins; MS (ESIpos) m/z=239 (M+H)⁺.

### Intermediate 3A: Ethyl 2-cyano-4-nitrobenzoate

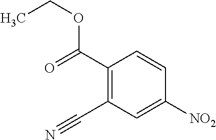 [SMILES: CCOC(=O)c1ccc([N+](=O)[O-])cc1C#N]

Ethyl 2-carbamoyl-4-nitrobenzoate (Int. 2A, 1.16 g, 4.9 mmol) was dissolved in thionyl chloride (11 mL, 146 mmol) and heated at 90° C. for 2 hours. The mixture was concentrated at reduced pressure and purified directly by Biotage Isolera™ chromatography (silica gel, eluting with heptane-EtOAc, 19:1 to 1:1) to afford 568 mg (53% yield) of the title compound as a white solid.

¹H NMR (500 MHz, DMSO-d6) δ[ppm] 8.82 (d, J=2.3 Hz, 1H), 8.60 (dd, J=8.7, 2.4 Hz, 1H), 8.32 (d, J=8.7 Hz, 1H), 4.43 (q, J=7.1 Hz, 2H), 1.37 (t, J=7.1 Hz, 3H).

LCMS (Analytical Method A): Rt=1.08 mins; MS (ESIneg) m/z=220 (M−H)⁻.

Alternatively, ethyl 2-chloro-4-nitrobenzoate (Int. 20A, 85.7 g, 373 mmol) was dissolved in NMP (180 mL), copper-(I)-cyanide (70.9 g, 791 mmol, 2.1 eq.) and tetrakis(triphenylphosphine)palladium (0) (86 mg, 75 μmol) were added, the mixture degassed and stirred for 16 h at 140° C. under an Argon atmosphere. After cooling the mixture was poured into water (3 L) with iron-(III)-chloride (60 g). Ethyl acetate (500 mL) was added and the mixture stirred for 15 min, then it was filtered through a pad of Celite®. The phases were separated, the aqueous layers were extracted with ethyl acetate and the combined organic layers washed with brine and dried with sodium sulphate. The solvents were evaporated and the residue purified by Biotage Isolera™ chromatography (silica gel, eluting with hex/EE 0-10-20%) to yield the title compound as a pale yellow solid (29.1 g, 35% yield). 40 g of the starting material (Ethyl 2-chloro-4-nitrobenzoate Int. 20A) was recovered from the reaction.

¹H NMR (400 MHz, DMSO-d6) δ[ppm] 1.37 (t, 3H), 4.43 (q, 2H), 8.32 (d, 1H), 8.61 (dd, 1H), 8.83 (d, 1H).

LCMS (method 6): Rt=0.92 min; MS (ESIpos) m/z=221 (M+H)⁺.

### Intermediate 4A: Ethyl 4-nitro-2-(1H-tetrazol-5-yl)benzoate

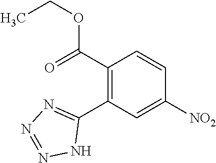 [SMILES: CCOC(=O)c1ccc([N+](=O)[O-])cc1-c1nnn[nH]1]

Ethyl 2-cyano-4-nitrobenzoate (Int. 3A, 0.582 g, 2.6 mmol) was dissolved in p-xylene (10 mL) and azidotrimethylsilane (0.42 mL, 3.1 mmol) and di-n-butyltin oxide (658 mg, 3.1 mmol) were added. The resulting mixture was heated in a sealed tube at 130° C. for 1 h and a yellow solution formed. MeOH (50 mL) was added and the mixture was stirred for 2 h until the material has fully dissolved. The mixture was then concentrated at reduced pressure and purified twice by Biotage Isolera™ chromatography (silica gel, eluting with DCM-MeOH, 49:1 to 7:3) to afford 220 mg (31% yield) of the title compound as a yellow solid.

¹H NMR (500 MHz, DMSO-d6) δ[ppm] 8.62 (d, J=2.3 Hz, 1H), 8.52 (dd, J=8.5, 2.3 Hz, 1H), 8.14 (d, J=8.5 Hz, 1H), 4.22 (q, J=7.1 Hz, 2H), 1.12 (t, J=7.1 Hz, 3H).

LCMS (Analytical Method A): Rt=0.97 mins; MS (ESIpos) m/z=264 (M+H)⁺.

### Intermediate 5A: Ethyl 4-amino-2-(1H-tetrazol-5-yl)benzoate

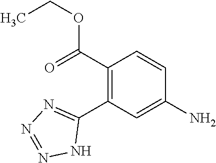 [SMILES: CCOC(=O)c1ccc(N)cc1-c1nnn[nH]1]

Ethyl 4-nitro-2-(1H-tetrazol-5-yl)benzoate (Int. 4A, 220 mg, 0.7 mmol) was dissolved in ethanol (20 mL) and 10% Palladium on carbon (15 mg) was added. The resulting mixture was stirred overnight under a hydrogen atmosphere. The mixture was filtered through Celite® then concentrated at reduced pressure. The product was purified by preparative HPLC (Method B) to afford 150 mg (77% yield) of the title compound as a white solid.

¹H NMR (500 MHz, DMSO-d6) δ[ppm] 7.77 (d, J=8.6 Hz, 1H), 6.76 (dd, J=8.6, 2.4 Hz, 1H), 6.64 (d, J=2.3 Hz, 1H), 6.25 (s, 2H), 4.00 (q, J=7.1 Hz, 2H), 1.02 (t, J=7.1 Hz, 3H).

LCMS (Analytical Method F): Rt=1.50 mins; MS (ESIpos) m/z=234 (M+H)⁺.

Alternatively, Ethyl 4-amino-2-cyanobenzoate (Int. 21A, 2.90 g, 15.2 mmol) was dissolved in toluene (120 mL) and azidotrimethylsilane (8.10 mL, 61.0 mmol) and di-n-butyltin oxide (5.69 g, 22.9 mmol) were added. The resulting mixture was stirred at 130° C. for 16 h then cooled to RT. MeOH was added to dissolve brown precipitate formed on cooling. The mixture was then concentrated at reduced pressure and purified twice by Biotage Isolera™ chromatography (silica gel, eluting with DCM-MeOH 0-10-30%) to give 1.90 g (49% yield) of the title compound as a brownish solid.

¹H NMR (400 MHz, DMSO-d6) δ[ppm] 1.02 (t, 3H), 4.00 (q, 2H), 6.28 (s, 2H), 6.64 (d, 1H), 6.76 (dd, 1H), 7.78 (d, 1H).

LCMS (method 1): Rt=0.51 min; MS (ESIpos) m/z=234 (M+H)⁺.

### Intermediate 6A: tert-butyl 5-nitro-1,3-dioxo-1,3-dihydro-2H-isoindole-2-carboxylate

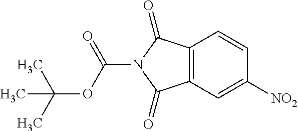 [SMILES: CC(C)(C)OC(=O)N1C(=O)c2ccc([N+](=O)[O-])cc2C1=O]

5-Nitro-1H-isoindole-1,3(2H)-dione (20 g, 105 mmol) and di-tert-butyl dicarbonate (23.8 g, 109.3 mmol) were stirred in acetonitrile (200 mL) and 4-dimethylaminopyridine (127 mg, 1.0 mmol) was added. Gas evolution was observed and the phthalimide slowly dissolved overnight. The following day the reaction was concentrated at reduced pressure to afford 29.8 g (quantitative yield) of the title compound as an orange solid.

¹H NMR (250 MHz, DMSO-d6) δ[ppm] 8.66 (dd, J=8.2, 2.0 Hz, 1H), 8.54 (d, J=1.7 Hz, 1H), 8.18 (d, J=8.2 Hz, 1H), 1.56 (s, 9H).

LCMS (Analytical Method A): Rt=1.16 mins, the title compound did not ionise.

### Intermediate 7A: Cyclopentyl 2-[(tert-butoxycarbonyl)carbamoyl]-4-nitrobenzoate

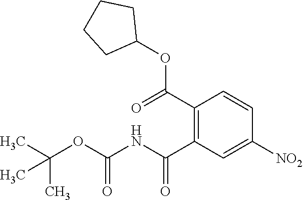 [SMILES: CC(C)(C)OC(=O)NC(=O)c1cc([N+](=O)[O-])ccc1C(=O)OC1CCCC1]

Tert-butyl 5-nitro-1,3-dioxo-1,3-dihydro-2H-isoindole-2-carboxylate (Int. 6A, 7.0 g, 23.9 mmol) was stirred in cyclopentanol (15 mL) and heated to 80° C. for 2 hours giving an orange solution. Crude ¹H NMR showed consumption of the starting material and formation of a 50:42:8 mixture of the two possible regioisomers and carboxylic acid. The mixture was concentrated at reduced pressure giving an orange solid. To this was added TBME (30 mL) and the mixture was slurried and sonicated for 5 mins. Heptane (80 mL) was then added to the mixture and the solids were collected via filtration and dried. The solids obtained were again slurried in TBME (15 mL) and heptane (60 mL) was added and the solids obtained were collected via filtration and dried. The filtrates were combined and concentrated at reduced pressure. The residue was purified by Biotage Isolera™ chromatography (silica gel, eluting with heptanes-TBME, 19:1 to 2:3) giving further product, which was slurried in TBME (10 mL) and heptane (50 mL). The solids were collected via filtration and dried and the resulting solids were again collected via filtration and dried. The batches were combined to afford 4.2 g (44% yield) of the title compound as a pale yellow solid.

¹H NMR (500 MHz, DMSO-d6) δ[ppm] 11.07 (s, 1H), 8.35 (dd, J=8.6, 2.3 Hz, 1H), 8.25 (d, J=2.2 Hz, 1H), 8.10 (d, J=8.6 Hz, 1H), 5.31 (m, 1H), 1.96-1.81 (m, 2H), 1.81-1.65 (m, 4H), 1.65-1.52 (m, 2H), 1.34 (s, 9H).

LCMS (Analytical Method A): Rt=1.23 mins; MS (ESIpos) m/z=401 (M+Na)⁺.

The following compound was synthesised in an analogous manner to Intermediate 7A, starting from Intermediate 6A and the appropriate alcohol:

| Int. | Structure | Name | Analytics |
|---|---|---|---|
| 8A | 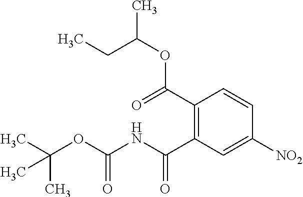 [SMILES: CCC(C)OC(=O)c1ccc([N+](=O)[O-])cc1C(=O)NC(=O)OC(C)(C)C] | sec-Butyl 2-[(tert- butoxycarbonyl) carbamoyl]-4- nitrobenzoate | ¹H NMR (500 MHz, DMSO-d6) δ [ppm] 11.03 (s, 1H), 8.36 (dd, J = 8.6, 2.3 Hz, 1H), 8.25 (d, J = 2.3 Hz, 1H), 8.11 (d, J = 8.6 Hz, 1H), 5.03- 4.92 (m, 1H), 1.69-1.58 (m, 2H), 1.33 (s, 9H), 1.25 (d, J = 6.3 Hz, 3H), 0.88 (t, J = 7.4 Hz, 3H). LCMS (Analytical Method A): Rt = 1.22 mins; MS (ESIpos) m/z = 389 (M + Na)⁺. |

### Intermediate 9A: Cyclopentyl 2-carbamoyl-4-nitrobenzoate

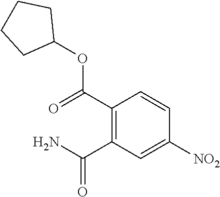 [SMILES: NC(=O)c1cc([N+](=O)[O-])ccc1C(=O)OC1CCCC1]

Cyclopentyl 2-[(tert-butoxycarbonyl)carbamoyl]-4-nitrobenzoate (Int 7A, 4.1 g, 10.1 mmol) was stirred in dichloromethane (20 mL) and trifluoroacetic acid (3.3 mL) was added at room temperature giving a pale yellow solution. The mixture was stirred for 5 hours until no further gas evolution was observed. The mixture was then concentrated at reduced pressure and dried overnight in the vacuum oven to afford 2.91 g (86% yield) of the title compound as an off-white solid.

¹H NMR (500 MHz, DMSO-d6) δ[ppm] 8.38-8.29 (m, 2H), 8.21 (s, 1H), 7.89 (d, J=8.4 Hz, 1H), 7.72 (d, J=6.2 Hz, 1H), 5.36-5.25 (m, 1H), 1.91-1.76 (m, 4H), 1.76-1.63 (m, 2H), 1.63-1.51 (m, 2H).

LCMS (Analytical Method A): Rt=1.00 mins; MS (ESIpos) m/z=279 (M+H)⁺.

The following compound was synthesised in an analogous manner to Intermediate 9A, from the appropriate Boc-protected starting material.

| Int. | Structure | Name | Analytics |
|---|---|---|---|
| 10A | 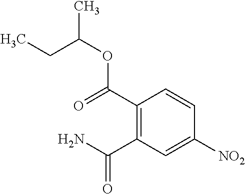 [SMILES: CCC(C)OC(=O)c1ccc([N+](=O)[O-])cc1C(N)=O] | sec-Butyl 2- carbamoyl-4- nitrobenzoate | ¹H NMR (500 MHz, DMSO-d6) δ [ppm] 8.40-8.32 (m, 2H), 8.22 (s, 1H), 7.88 (d, J = 8.2 Hz, 1H), 7.70 (s, 1H), 5.01-4.90 (m, 1H), 1.71- 1.55 (m, 2H), 1.27 (d, J = 6.3 Hz, 3H), 0.91 (t, J = 7.5 Hz, 3H). LCMS (Analytical Method A): Rt = 0.99 mins; MS (ESIpos) m/z = 267 (M + H)⁺. |

### Intermediate 11A: Cyclopentyl 2-cyano-4-nitrobenzoate

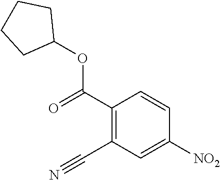 [SMILES: N#Cc1cc([N+](=O)[O-])ccc1C(=O)OC1CCCC1]

Cyclopentyl 2-carbamoyl-4-nitrobenzoate (Int. 9A, 2.91 g, 10.5 mmol) was dissolved in thionyl chloride (15 mL, 209 mmol) and heated at 65° C. for 2 hours and then 90° C. for 3 hours, during which time the starting material slowly dissolved giving a dark orange solution. The mixture was concentrated at reduced pressure and purified by Biotage Isolera™ chromatography (silica gel, eluting with heptanes-EtOAc, 49:1 to 1:4) to afford 1.38 g (51% yield) of the title compound as a yellow solid.

¹H NMR (250 MHz, DMSO-d6) δ[ppm] 8.82-8.77 (m, 1H), 8.62-8.55 (m, 1H), 8.36-8.27 (m, 1H), 5.52-5.34 (m, 1H), 2.13-1.54 (m, 8H).

LCMS (Analytical Method A): Rt=1.23 mins; MS (ESIneg): m/z=260 (M−H)⁻.

The following compound was synthesised in an analogous manner to Intermediate 11A, from the appropriate carbamoyl starting material.

| Int. | Structure | Name | Analytics |
|---|---|---|---|
| 12A | 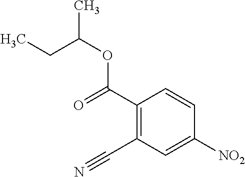 [SMILES: CCC(C)OC(=O)c1ccc([N+](=O)[O-])cc1C#N] | sec-Butyl 2- cyano-4- nitrobenzoate | ¹H NMR (500 MHz, DMSO-d6) δ [ppm] 8.80 (d, J = 2.3 Hz, 1H), 8.59 (dd, J = 8.7, 2.4 Hz, 1H), 8.31 (d, J = 8.6 Hz, 1H), 5.17-5.04 (m, 1H), 1.82-1.64 (m, 2H), 1.35 (d, J = 6.3 Hz, 3H), 0.95 (t, J = 7.5 Hz, 3H). LCMS (Analytical Method A): Rt = 1.22 mins, the title compound did not ionise. |

### Intermediate 13A: Cyclopentyl 4-nitro-2-(1H-tetrazol-5-yl)benzoate

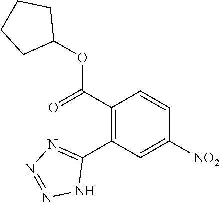 [SMILES: O=C(OC1CCCC1)c1ccc([N+](=O)[O-])cc1-c1nnn[nH]1]

Cyclopentyl 2-cyano-4-nitrobenzoate (Int. 11A, 1.38 g, 5.3 mmol) was dissolved in p-xylene (10 mL) and azidotrimethylsilane (0.84 mL, 6.3 mmol) and di-n-butyltin oxide (1.32 g, 5.30 mmol) were added. The resulting mixture heated at 130° C. for 1.5 hours giving a yellow solution. Further azidotrimethylsilane (0.42 mL, 3.2 mmol) was added and the mixture was heated at 130° C. for a further 1.5 hours and an off white precipitate formed. MeOH (50 mL) was added and the mixture was stirred for 15 minutes and then concentrated at reduced pressure. The residue was slurried in 3:1 DCM/MeOH (30 mL) and the solids were filtered and removed. The filtrate was concentrated at reduced pressure and purified by Biotage Isolera™ chromatography (silica gel, eluting with DCM-MeOH, 49:1 to 7:3) to afford 920 mg (40% yield) of the title compound as a pale yellow solid.

¹H NMR (250 MHz, DMSO-d6) δ[ppm] 8.58 (d, J=2.2 Hz, 1H), 8.50 (dd, J=8.5, 2.3 Hz, 1H), 8.12 (d, J=8.5 Hz, 1H), 5.30-5.17 (m, 1H), 1.89-1.66 (m, 2H), 1.66-1.35 (m, 6H).

LCMS (Analytical Method A): Rt=1.07 mins; MS (ESIpos) m/z=304 (M+H)⁺.

The following compound was synthesised in an analogous manner to Intermediate 13A, from the appropriate nitrile starting material.

| Int. | Structure | Name | Analytics |
|---|---|---|---|
| 14A | 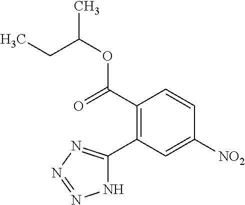 [SMILES: CCC(C)OC(=O)c1ccc([N+](=O)[O-])cc1-c1nnn[nH]1] | sec-Butyl 4-nitro- 2-(1H-tetrazol-5- yl)benzoate | ¹H NMR (500 MHz, DMSO-d6) δ [ppm] 8.60 (d, J = 2.3 Hz, 1H), 8.46 (dd, J = 8.5, 2.4 Hz, 1H), 8.05 (d, J = 8.5 Hz, 1H), 4.98-4.86 (m, 1H), 1.52-1.42 (m, 2H), 1.14 (d, J = 6.3 Hz, 3H), 0.77 (t, J = 7.5 Hz, 3H). LCMS (Analytical Method A): Rt = 1.07 mins; MS (ESIpos) m/z = 292 (M + H)⁺. |

### Intermediate 15A: 4-Nitro-2-(1H-tetrazol-5-yl)benzoic acid

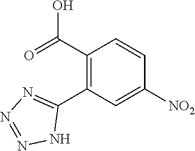 [SMILES: O=C(O)c1ccc([N+](=O)[O-])cc1-c1nnn[nH]1]

sec-Butyl 4-nitro-2-(1H-tetrazol-5-yl)benzoate (Int. 14A, 3.08 g, 10.6 mmol) was dissolved in tetrahydrofuran (30 mL) and to this was added lithium hydroxide monohydrate (2.66 g, 63.4 mmol) in water (30 mL). This was rapidly stirred overnight and the following day complete saponification was observed. The mixture was acidified with 1M aq. HCl (5 mL) and then diluted with water (20 mL) and EtOAc (50 mL). The organic layer was then extracted and washed with brine (2×20 mL), dried (Na₂SO₄), filtered and dried giving the desired product (2.10 g, 85% yield) as a yellow solid. 1H NMR (500 MHz, DMSO-d6) δ 8.55 (d, J=2.3 Hz, 1H), 8.50 (dd, J=8.5, 2.4 Hz, 1H), 8.16 (d, J=8.5 Hz, 1H).

LCMS (Analytical Method A): Rt=0.81 mins; MS (ESIPos): m/z=236 (M+H)+

### Intermediate 16A: N-(Cyclohexylmethyl)-N-methyl-4-nitro-2-(1H-tetrazol-5-yl)benzamide

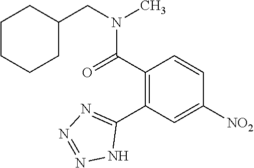 [SMILES: CN(CC1CCCCC1)C(=O)c1ccc([N+](=O)[O-])cc1-c1nnn[nH]1]

4-Nitro-2-(1H-tetrazol-5-yl)benzoic acid (Int. 15A, 200 mg, 0.85 mmol) and 1-cyclohexyl-N-methylmethanamine (0.162 mg, 1.28 mmol) were dissolved in DMF (4 mL) and N,N-diisopropylethylamine (0.5 mL, 2.551 mmol) and HATU (0.388 g, 1.021 mmol) were added giving an orange solution. The mixture was stirred overnight at room temperature. The following day the mixture was diluted with dichloromethane (2 mL) and washed with 1 M aq. HCl (2×2 mL) and brine (2 mL), dried (MgSO₄), filtered and concentrated at reduced pressure. The residue was purified via preparative HPLC (Method B) giving the product (340 mg, 99% yield) as a white solid.

LCMS (Analytical Method A): Rt=1.11 mins; MS (ESIPos): m/z=345 (M+H)⁺.

### Intermediate 17A: Cyclopentyl 4-amino-2-(1H-tetrazol-5-yl)benzoate

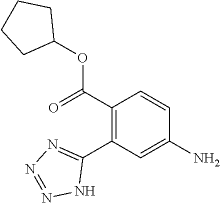 [SMILES: Nc1ccc(C(=O)OC2CCCC2)c(-c2nnn[nH]2)c1]

Cyclopentyl 4-nitro-2-(1H-tetrazol-5-yl)benzoate (Int. 13A, 920 mg, 2.12 mmol, 70% purity) was dissolved in ethyl acetate (30 mL) and 10% Palladium on carbon (22 mg) was added and the resulting mixture was stirred under a hydrogen atmosphere overnight. The mixture was filtered through Celite® (washing with methanol) and then concentrated at reduced pressure. The residue was then purified by Biotage Isolera™ chromatography (silica gel, eluting with DCM-MeOH, 49:1 to 7:3). The material was then purified via preparative HPLC (Method B) to afford 327 mg (56% yield) of the title compound as a white solid.

¹H NMR (500 MHz, DMSO-d6) δ[ppm] 7.76 (d, J=8.6 Hz, 1H), 6.76 (dd, J=8.7, 2.3 Hz, 1H), 6.59 (d, J=2.3 Hz, 1H), 6.23 (s, 2H), 5.07-4.95 (m, 1H), 1.74-1.62 (m, 2H), 1.50-1.31 (m, 6H).

LCMS (Analytical Method F): Rt=2.16 mins; MS (ESIpos) m/z=274 (M+H)⁺.

The following compounds were synthesised in an analogous manner to Intermediate 17A, from the appropriate nitro starting material.

| Int. | Structure | Name | Analytics |
|---|---|---|---|
| 18A | 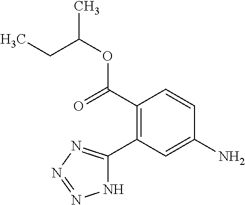 [SMILES: CCC(C)OC(=O)c1ccc(N)cc1-c1nnn[nH]1] | sec-Butyl 4- amino-2-(1H- tetrazol-5- yl)benzoate | ¹H NMR (250 MHz, DMSO-d6) δ [ppm] 7.78 (d, J = 8.6 Hz, 1H), 6.77 (dd, J = 8.6, 2.3 Hz, 1H), 6.60 (d, J = 2.3 Hz, 1H), 6.24 (s, 2H), 4.79- 4.58 (m, 1H), 1.45-1.23 (m, 2H), 1.01 (d, J = 6.2 Hz, 3H), 0.72 (t, J = 7.4 Hz, 3H). LCMS (Analytical Method A): Rt = 0.93 mins; MS (ESIpos) m/z = 262 (M + H)⁺. |
| 19A | 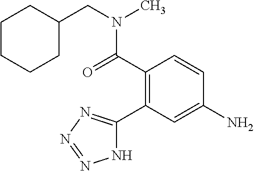 [SMILES: CN(CC1CCCCC1)C(=O)c1ccc(N)cc1-c1nnn[nH]1] | 4-amino-N- (cyclohexylmethyl)- N-methyl-2-(1H- tetrazol-5- yl)benzamide | ¹H NMR (500 MHz, DMSO-d6) δ 6.99 (m, 2H), 6.65 (s, 1H), 3.18- 2.62 (m, 5H), 1.77-1.35 (m, 6H), 1.35-0.40 (m, 5H). LCMS (Analytical Method D): Rt = 3.47 mins, MS (ESIpos) m/z = 315 (M + H)⁺. |

### Intermediate 20A: Ethyl 2-chloro-4-nitrobenzoate

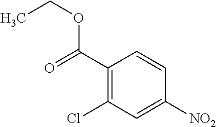

2-Chloro-4-nitrobenzoic acid (80.0 g, 397 mmol) was dissolved in ethanol (1200 mL) and sulfuric acid (1 eq., 21.2 mL) was added drop wise. The mixture was stirred at 120° C. for 24 h. After cooling the mixture was concentrated in vacuo to ⅓ of the original volume, diluted with water and extracted with ethyl acetate. The combined organic layers were washed with saturated aqueous NaHCO₃ solution and brine, dried (Na₂SO₄), filtered and the solvents evaporated. The title compound was obtained as a yellow oil (85.7 g, 94% yield) which was used without further purification.

¹H NMR (400 MHz, DMSO-d6) δ[ppm] 1.34 (t, 3H), 4.39 (q, 2H), 8.03 (d, 1H), 8.28 (dd, 1H), 8.39 (d, 1H).

LCMS (method 6): Rt=1.12 min; MS (ESIpos) m/z=230 (M+H)⁺.

### Intermediate 21A: Ethyl 4-amino-2-cyanobenzoate

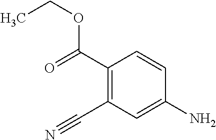

Ethyl 2-cyano-4-nitrobenzoate (Int. 3A, 27.6 g, 125 mmol) was dissolved in ethyl acetate (600 mL) and palladium (10% on activated carbon; 13 g, 12.5 mmol, 0.1 eq.) was added. The mixture was shaken at RT under hydrogen atmosphere until complete conversion (ca. 2 h). The catalyst was filtered off, rinsed with ethyl acetate and the filtrate concentrated to dryness in vacuo to yield 2.9 g (12% yield) of the pure title compound as an off-white solid. The used Pd-catalyst was suspended in DMF, extracted and filtered off again three times. The DMF solutions were combined and the solvent evaporated in vacuo. A further 19 g (68% yield) of the title compound was obtained as a grey solid (purity ca. 85%), which was used without further purification.

¹H NMR (400 MHz, DMSO-d6) δ[ppm] 1.29 (t, 3H), 4.26 (q, 2H), 6.46 (s, 2H), 6.82 (dd, 1H), 6.95 (d, 1H), 7.78 (d, 1H).

LCMS (method 1): Rt=0.74 min; MS (ESIpos) m/z=191 (M+H)⁺.

### Intermediate 22A: 1-(3-Chlorophenyl)cyclopropanecarbonyl chloride

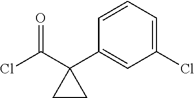 [SMILES: O=C(Cl)C1(c2cccc(Cl)c2)CC1]

1-(3-Chlorophenyl)cyclopropanecarboxylic acid (2.0 g, 10.1 mmol) was stirred in thionyl chloride (12 mL) and DMF (2 drops) was added. The mixture was stirred at room temperature for 5 hours. NMR (sample prepared from MeOH) shows complete conversion to the methyl ester. The mixture was then concentrated at reduced pressure. The residue was diluted with heptane (3 mL) and re-concentrated twice to remove excess thionyl chloride to afford 1.94 g (87% yield) of the title compound as a yellow oil.

Analysis of methyl ester: ¹H NMR (250 MHz, DMSO-d6) δ[ppm] 7.45-7.22 (m, 4H), 3.55 (s, 3H), 1.56-1.40 (m, 2H), 1.30-1.15 (m, 2H).

The following acid chlorides were prepared in an analogous manner to Intermediate 22A, from the appropriate carboxylic acids.

| Int. | Structure | Name | Analytics |
|---|---|---|---|
| 23A | 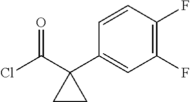 [SMILES: O=C(Cl)C1(c2ccc(F)c(F)c2)CC1] | 1-(3,4- Difluorophenyl) cyclopropane- carbonyl chloride | NMR of methyl ester: ¹H NMR (250 MHz, d6-DMSO) δ [ppm] 7.50- 7.24 (m, 2H), 7.19 (m, 1H), 3.55 (s, 3H), 1.53-1.43 (m, 2H), 1.27- 1.16 (m, 2H). |
| 24A | 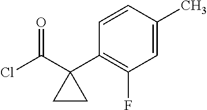 | 1-(2-Fluoro-4- methylphenyl)cyclo- propanecarbonyl chloride | ¹H NMR (250 MHz, Chloroform-d) δ [ppm] 1.31-1.43 (m, 2H), 1.89 (m, 2H), 2.27 (s, 3H), 6.83 (m, 2H), 6.97-7.22 (m, 1H). |
| 25A | 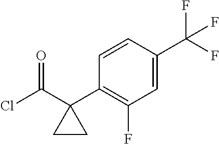 [SMILES: O=C(Cl)C1(c2ccc(C(F)(F)F)cc2F)CC1] | 1-[2-Fluoro-4- (trifluoromethyl) phenyl]cyclopropane- carbonyl chloride | NMR of methyl ester: ¹H NMR (500 MHz, DMSO-d6) δ [ppm] 7.71- 7.49 (m, 4H), 3.57 (s, 3H), 1.61- 1.54 (m, 2H), 1.35-1.28 (m, 2H). |
| 26A | 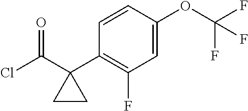 [SMILES: O=C(Cl)C1(c2ccc(OC(F)(F)F)cc2F)CC1] | 1-[2-Fluoro-4- (trifluoromethoxy) phenyl]cyclopropane- carbonyl chloride | ¹H NMR (500 MHz, Chloroform-d) δ [ppm] 7.37-7.28 (m, 1H), 7.07- 6.86 (m, 2H), 2.10-1.96 (m, 2H), 1.52-1.40 (m, 2H). |

### Intermediate 27A: 2-Bromo-3-fluoro-5-nitrobenzonitrile

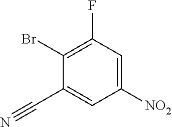 [SMILES: N#Cc1cc([N+](=O)[O-])cc(F)c1Br]

To a solution of 2-bromo-3-fluorobenzonitrile (9.94 g, 49.7 mmol) in sulfuric acid (20 mL) was added nitric acid (69%, 4.8 mL, 75 mmol) dropwise, maintaining the internal temperature between 5-10° C. The reaction mixture turned brown at the end of the addition. The reaction mixture was allowed to warm up to room temperature and was stirred overnight resulting in formation of a light orange precipitate. The reaction mixture was poured onto crushed ice. The products were extracted with EtOAc (2×100 mL). The combined organic layers were washed with brine (50 mL), dried (MgSO₄), filtered and concentrated under reduced pressure. The residue was absorbed onto silica and then passed through a silica plug (eluting with 7:3 heptane/EtOAc) to remove insoluble baseline impurities. The residue was then purified by Biotage Isolera™ chromatography (silica gel, eluting with 10-40% EtOAc in heptane) to give the title compound (3.69 g, 30% yield) as a pale yellow solid.

¹H NMR (500 MHz, DMSO-d6) δ[ppm] 8.77 (dd, J=2.5, 1.4 Hz, 1H), 8.65 (dd, J=8.3, 2.5 Hz, 1H).

LCMS (Analytical Method A): Rt=1.11 min, mass ion not observed.

The following Intermediate compound was synthesised in an analogous manner to Intermediate 27A, starting from the appropriate benzonitrile starting material:

| Int. | Structure | Name | Analytics |
|---|---|---|---|
| 28A | 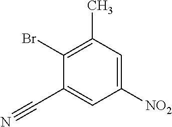 | 2-Bromo-3- methyl-5- nitrobenzonitrile | ¹H NMR (500 MHz, DMSO-d6) δ 8.69-8.63 (m, 1H), 8.57-8.52 (m, 1H), 2.54 (s, 3H) LCMS (Analytical Method A): Rt = 1.14 mins; the product did not ionize. |

### Intermediate 29A: 5-Amino-2-bromo-3-fluorobenzonitrile

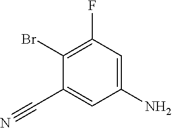 [SMILES: N#Cc1cc(N)cc(F)c1Br]

Iron powder (9.1 g 163 mmol) was added to a solution of 2-bromo-3-fluoro-5-nitrobenzonitrile (Int. 27A, 4.0 g, 16.3 mmol) in ethanol (30 mL) and acetic acid (5 mL) and the resulting mixture was heated at 90° C. for 1 hour. The mixture was filtered over a silica plug (washing with 50 mL EtOAc) then dried and concentrated at reduced pressure. The residue was purified by Biotage Isolera™ chromatography (silica gel, eluting with 98:2 to 4:6 heptane/EtOAc) to give the title compound (2.5 g, 72% yield) as an off white solid.

¹H NMR (500 MHz, DMSO-d6) δ[ppm] 6.86 (dd, J=2.5, 0.7 Hz, 1H), 6.77 (dd, J=11.5, 2.5 Hz, 1H), 6.10 (s, 2H).

LCMS (Analytical Method A): Rt=1.11 mins, mass ion not observed.

### Intermediate 30A: 5-amino-2-bromo-3-methylbenzonitrile

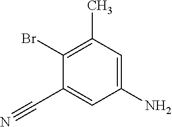

2-Bromo-3-methyl-5-nitrobenzonitrile (Int. 28A, 7.2 g, 25.4 mmol) was dissolved in ethanol (50 mL) and Iron powder (14.1 g, 253 mmol) and conc. hydrochloric acid (5 drops) were added. The resulting mixture was stirred at 90° C. for 2 days. The mixture was then cooled to room temperature and filtered over Celite (washing with methanol (200 mL)) then dried and concentrated at reduced pressure. The residue was purified via silica flash column chromatography (using a gradient of eluents; 9:1 to 1:1 heptane:EtOAc) giving the desired product (1.42 g, 27% yield) as a yellow solid. 1H NMR (500 MHz, DMSO-d6) δ 6.87-6.76 (m, 2H), 5.67 (s, 2H), 3.31 (s, 3H) LCMS (Analytical Method A): Rt=1.05 mins; MS (ESIPos): m/z=212 (M+H)⁺.

### Intermediate 31A: N-(4-bromo-3-cyano-5-fluorophenyl)-1-[2-fluoro-4-trifluoromethyl)-phenyl]cyclopropanecarboxamide

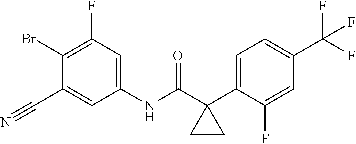 [SMILES: N#Cc1cc(NC(=O)C2(c3ccc(C(F)(F)F)cc3F)CC2)cc(F)c1Br]

5-Amino-2-bromo-3-fluorobenzonitrile (Int. 29A, 1.0 g, 4.65 mmol) was stirred in dichloromethane (30 mL) and added to 1-[2-fluoro-4-(trifluoromethyl)phenyl]cyclopropane-carbonyl chloride (1.36 g, 5.11 mmol) in pyridine (1.9 mL) at room temperature giving a dark brown solution. The mixture was stirred for 30 minutes at room temperature. The mixture was then diluted with ethyl acetate (30 mL) and washed with 1M aq. HCl (2×20 mL), brine (20 mL), dried (Na₂SO₄), filtered and concentrated at reduced pressure. The residue was purified by Biotage Isolera™ chromatography (silica gel, eluting with 95:5 to 1:1 heptane/EtOAc) to give the title compound (1.9 g, 91% yield) as a colourless oil.

¹H NMR (500 MHz, DMSO-d6) δ[ppm] 9.41 (s, 1H), 8.00-7.91 (m, 2H), 7.72-7.63 (m, 2H), 7.61 (d, J=8.1 Hz, 1H), 1.73-1.61 (m, 2H), 1.33-1.24 (m, 2H).

LCMS (Analytical Method A): Rt=1.40 mins; MS (ESIPos): m/z=445 (M+H)⁺.

The following compound was synthesised in an analogous manner to Intermediate 31A, starting from the appropriate aniline and acid chloride starting materials.

| Ex. | Structure | Name | Analytics |
|---|---|---|---|
| 32A | 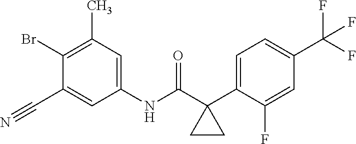 [SMILES: Cc1cc(NC(=O)C2(c3ccc(C(F)(F)F)cc3F)CC2)cc(C#N)c1Br] | N-(4-bromo-3- cyano-5- methylphenyl)-1- [2-fluoro-4- (trifluorometh- yl)phenyl]cyclo- propane- carboxamide | ¹H NMR (500 MHz, DMSO-d6) δ 9.22 (s, 1H), 7.92 (d, J = 2.5 Hz, 1H), 7.81 (d, J = 2.4 Hz, 1H), 7.72-7.63 (m, 2H), 7.60 (d, J = 8.1 Hz, 1H), 2.35 (s, 3H), 1.69- 1.60 (m, 2H), 1.31-1.22 (m, 2H). LCMS (Analytical Method A): Rt = 1.41 mins; MS (ESIPos): |
| | | | m/z = 443 (M + H)⁺. |

### Intermediate 33A: Ethyl 2-cyano-6-fluoro-4-[({1-[2-fluoro-4-(trifluoromethyl)phenyl]-cyclopropyl}carbonyl)amino]benzoate

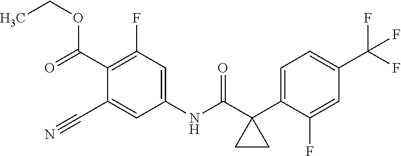 [SMILES: CCOC(=O)c1c(F)cc(NC(=O)C2(c3ccc(C(F)(F)F)cc3F)CC2)cc1C#N]

N-(4-bromo-3-cyano-5-fluorophenyl)-1-[2-fluoro-4-trifluoromethyl)phenyl]cyclcopropane-carboxamide (Int. 31A, 1.96 g, 4.4 mmol) was stirred in ethanol (30 mL) and triethylamine (3.7 mL, 26.4 mmol) and 1,1′-Bis(diphenylphosphino)ferrocenepalladium(II) chloride (161 mg, 0.22 mmol) were added. The resulting mixture was heated at 80° C. in a sealed vessel under 100 psi pressure of carbon monoxide for 18 hours. The reaction mixture was filtered through Celite®, concentrated at reduced pressure and purified by Biotage Isolera™ chromatography (silica gel, eluting with 98:2 to 1:1 heptane/EtOAc) to give the title compound (960 mg, 65% yield) as a white solid.

¹H NMR (500 MHz, DMSO-d6) δ[ppm] 9.57 (s, 1H), 8.03-7.90 (m, 2H), 7.75-7.64 (m, 2H), 7.62 (d, J=8.0 Hz, 1H), 4.36 (q, J=7.1 Hz, 2H), 1.73-1.64 (m, 2H), 1.36-1.27 (m, 5H).

LCMS (Analytical Method A): Rt=1.41 mins, MS (ESIPos): m/z=439 (M+H)+.

The following compound was synthesised in an analogous manner to Intermediate 33A, starting from the appropriate aryl bromide starting material.

| Ex. | Structure | Name | Analytics |
|---|---|---|---|
| 34A | 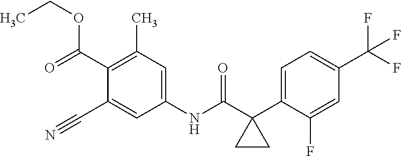 [SMILES: CCOC(=O)c1c(C)cc(NC(=O)C2(c3ccc(C(F)(F)F)cc3F)CC2)cc1C#N] | Ethyl 2-cyano-4- [({1-[2-fluoro-4- (trifluorometh- yl)phenyl]cyclo- propyl}carbonyl) amino]- 6-methylbenzoate | ¹H NMR (500 MHz, DMSO-d6) δ 9.33 (s, 1H), 7.97 (d, J = 2.1 Hz, 1H), 7.80 (d, J = 2.0 Hz, 1H), 7.73-7.65 (m, 2H), 7.61 (d, J = 8.1 Hz, 1H), 4.36 (q, J = 7.1 Hz, 2H), 2.37 (s, 3H), 1.69-1.62 (m, 2H), 1.33 (t, J = 7.1 Hz, 3H), 1.30- 1.24 (m, 2H). LCMS (Analytical Method A): Rt = 1.41 mins, MS (ESIPos): |
| | | | m/z = 435 (M + H)⁺ |

### Intermediate 35A: 4-({[1-(3-Chlorophenyl)cyclopropyl]carbonyl}amino)-2-(1H-tetrazol-5-yl)benzoic acid

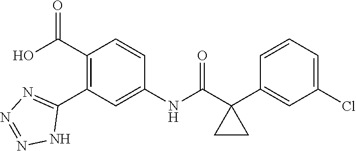 [SMILES: O=C(O)c1ccc(NC(=O)C2(c3cccc(Cl)c3)CC2)cc1-c1nnn[nH]1]

Ethyl 4-({[1-(3-chlorophenyl)cyclopropyl]carbonyl}amino)-2-(1H-tetrazol-5-yl)benzoate (Example 1, 55 mg, 0.13 mmol) was dissolved in tetrahydrofuran (2 mL) and to this was added lithium hydroxide monohydrate (33.6 mg, 0.8 mmol) in water (2 mL). This was rapidly stirred overnight. The mixture was acidified with 1M aq. HCl (3 mL) and then diluted with water (20 mL) and EtOAc (30 mL). The organic layer was then extracted and washed with brine (2×20 mL), dried (Na₂SO₄), filtered and dried to afford 40 mg (78% yield) of the title compound as a white solid.

¹H NMR (500 MHz, DMSO-d6) δ[ppm] 9.67 (s, 1H), 8.02-7.98 (m, 1H), 7.98-7.93 (m, 1H), 7.89-7.85 (m, 1H), 7.46-7.42 (m, 1H), 7.41-7.33 (m, 3H), 1.55-1.45 (m, 2H), 1.22-1.19 (m, 2H).

LCMS (Analytical Method F): Rt=2.82 mins; MS (ESIpos) m/z=384 (M+H)+.

### Intermediate 36A: 4-[({1-[2-fluoro-4-(trifluoromethyl)phenyl]cyclopropyl}carbonyl)amino]-2-(1H-tetrazol-5-yl)benzoic acid

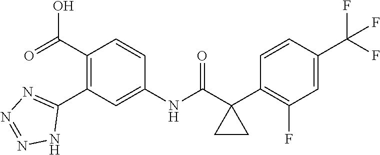 [SMILES: O=C(O)c1ccc(NC(=O)C2(c3ccc(C(F)(F)F)cc3F)CC2)cc1-c1nnn[nH]1]

sec-butyl 4-[({1-[2-fluoro-4-(trifluoromethyl)phenyl]cyclopropyl}carbonyl)amino]-2-(1H-tetrazol-5-yl)benzoate (Example 10, 2.14 g, 4.4 mmol) was dissolved in tetrahydrofuran (10 mL) and to this was added lithium hydroxide monohydrate (2.2 g, 52 mmol) in water (25 mL). This was stirred at 65° C. for 6 h. The mixture was acidified with 2M aq. HCl (35 mL) and the aqueous mixture extracted with ethyl acetate (75 mL). The organic layer was washed with brine (2×50 mL), dried (Na₂SO₄), filtered and concentrated at reduced pressure to give 1.79 g (94% yield) of the title compound as a pale orange solid.

¹H NMR (500 MHz, DMSO-d6) δ[ppm] 9.39 (s, 1H), 8.02-7.97 (m, 1H), 7.97-7.93 (m, 1H), 7.82-7.78 (m, 1H), 7.73-7.65 (m, 2H), 7.62-7.58 (m, 1H), 1.71-1.61 (m, 2H), 1.30-1.25 (m, 2H).

LCMS (Analytical Method F): Rt=3.06 mins; MS (ESIPos) m/z=436 (M+H)⁺.

The following Intermediate compound was synthesised in an analogous manner to Intermediate 36A, starting from the appropriate ester starting material.

| Int. | Structure | Name | Analytics |
|---|---|---|---|
| 37A | 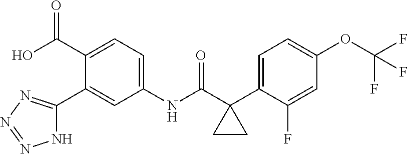 [SMILES: O=C(O)c1ccc(NC(=O)C2(c3ccc(OC(F)(F)F)cc3F)CC2)cc1-c1nnn[nH]1] | 4-[({1-[2-fluoro- 4- (trifluoromethoxy) phenyl]cyclopropyl} carbonyl)amino]- 2-(1H-tetrazol-5- yl)benzoic acid | ¹H NMR (500 MHz, DMSO-d6) δ 9.35 (s, 1H), 8.03-7.89 (m, 2H), 7.80 (d, J = 1.8 Hz, 1H), 7.66-7.55 (m, 1H), 7.41-7.33 (m, 1H), 7.28-7.19 (m, 1H), 1.67-1.56 (m, 2H), 1.27-1.18 (m, 2H). LCMS (Analytical Method F): Rt = 3.15 mins; MS (ESIPos): m/z = 452 (M + H)⁺ |

### Intermediate 38A: 2-fluoro-4-[({1-[2-fluoro-4-(trifluoromethyl)phenyl]cyclopropyl}-carbonyl)amino]-6-(1H-tetrazol-5-yl)benzoic acid

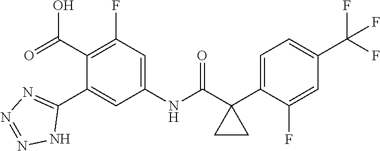 [SMILES: O=C(O)c1c(F)cc(NC(=O)C2(c3ccc(C(F)(F)F)cc3F)CC2)cc1-c1nnn[nH]1]

Ethyl 2-fluoro-4-[({1-[2-fluoro-4-(trifluoromethyl)phenyl]cyclopropyl}carbonyl)amino]-6-(1H-tetrazol-5-yl)benzoate (Example 59, 510 mg, 1.06 mmol) was dissolved in THF (15 mL) and lithium hydroxide monohydrate (800 mg, 19.0 mmol) in water (15 mL) was added. The mixture was heated at 90° C. for 12 hours. The reaction was then cooled to room temperature and acidified with 2M aq. HCl (20 mL) and then diluted with ethyl acetate (40 mL). The aqueous layer was extracted and the organics were then washed with brine (20 mL), dried (Na₂SO₄), filtered and concentrated at reduced pressure. The residue was purified via acidic prep HPLC (Method B) giving the title compound (156 mg, 33% yield) as an off white solid.

¹H NMR (500 MHz, DMSO-d6) δ[ppm] 9.47 (s, 1H), 7.91-7.76 (m, 2H), 7.75-7.66 (m, 2H), 7.65-7.59 (m, 1H), 1.72-1.63 (m, 2H), 1.33-1.24 (m, 2H).

LCMS (Analytical Method A): Rt=1.22 mins, MS (ESIPos): m/z=454 (M+H)⁺.

### Intermediate 39A: 4-[({1-[2-fluoro-4-(trifluoromethyl)phenyl]cyclopropyl}carbonyl)amino]-2-methyl-6-(1H-tetrazol-5-yl)benzoic acid

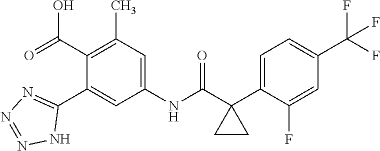 [SMILES: Cc1cc(NC(=O)C2(c3ccc(C(F)(F)F)cc3F)CC2)cc(-c2nnn[nH]2)c1C(=O)O]

Ethyl 4-[({1-[2-fluoro-4-(trifluoromethyl)phenyl]cyclopropyl}carbonyl)amino]-2-methyl-6-(1H-tetrazol-5-yl)benzoate (Example 60, 540 mg, 1.13 mmol) was dissolved in pyridine (10 mL) and Lithium iodide (1.2 g, 9.0 mmol) and water (163 uL, 9.0 mmol) were added. The mixture was heated in a sealed tube at 120° C. for 3 days. The mixture was then cooled to room temperature and concentrated at reduced pressure to remove excess pyridine. The residue was diluted with ethyl acetate (60 mL) and washed with 1M aq. HCl (2×30 mL) and brine (30 mL), dried (Na₂SO₄), filtered and concentrated at reduced pressure. The residue was purified via preparative LC (Method B) giving the desired product (211 mg, 41% yield) as an off white solid.

1H NMR (500 MHz, DMSO-d6) δ 9.21 (s, 1H), 7.90-7.78 (m, 1H), 7.75-7.64 (m, 2H), 7.64-7.52 (m, 2H), 2.34 (s, 3H), 1.68-1.57 (m, 2H), 1.28-1.15 (m, 2H).

LCMS (Analytical Method F): Rt=3.10 mins, MS (ESIPos): m/z=450 (M+H)⁺.

### Example 1: Ethyl 4-({[1-(3-chlorophenyl)cyclopropyl]carbonyl}amino)-2-(1H-tetrazol-5-yl)benzoate

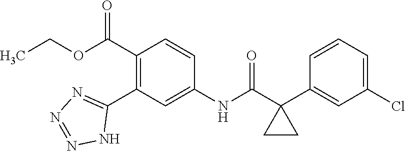 [SMILES: CCOC(=O)c1ccc(NC(=O)C2(c3cccc(Cl)c3)CC2)cc1-c1nnn[nH]1]

Ethyl 4-amino-2-(1H-tetrazol-5-yl)benzoate (Int. 5A, 360 mg, 1.54 mmol) and 1-(3-chlorophenyl)cyclopropane-1-carboxylic acid (364 mg, 1.85 mmol) were dissolved in DMF (3 mL) and N,N-diisopropylethylamine (0.81 mL, 4.6 mmol), 4-dimethylaminopyridine (68 mg, 0.56 mmol) and HATU (705 mg, 1.85 mmol) were added. This was stirred for 3 days at RT. The mixture was concentrated at reduced pressure to remove excess DMF then diluted with EtOAc (50 mL) and washed with 1M aq. HCl (20 mL), brine (2×20 mL), dried (Na₂SO₄), filtered and concentrated at reduced pressure. The residue was purified by Biotage Isolera™ chromatography (silica gel, eluting with DCM-MeOH, 49:1 to 4:1), then re-purified by preparative HPLC (Method B) to afford 60 mg (9% yield) of the title compound as a yellow solid.

¹H NMR (500 MHz, DMSO-d6) δ[ppm] 9.69 (s, 1H), 8.02-7.85 (m, 3H), 7.46-7.30 (m, 4H), 4.09 (q, J=7.1 Hz, 2H), 1.53-1.47 (m, 2H), 1.25-1.17 (m, 2H), 1.05 (t, J=7.1 Hz, 3H).

LCMS (Analytical Method F): Rt=3.29 mins; MS (ESIpos) m/z=412 (M+H)⁺.

### Example 2: Cyclopentyl 4-({[1-(3-chlorophenyl)cyclopropyl]carbonyl}amino)-2-(1H-tetrazol-5-yl)benzoate

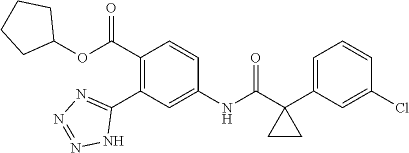 [SMILES: O=C(OC1CCCC1)c1ccc(NC(=O)C2(c3cccc(Cl)c3)CC2)cc1-c1nnn[nH]1]

4-({[1-(3-Chlorophenyl)cyclopropyl]carbonyl}amino)-2-(1H-tetrazol-5-yl)benzoic acid (Int. 35A, 30 mg, 0.08 mmol) was dissolved in cyclopentanol (1.5 mL) and concentrated sulfuric acid (1 drop) was added. The mixture was heated at 75° C. overnight then allowed to cool to room temperature before being diluted with ethyl acetate (10 mL) and washed with brine (3 mL), dried (Na₂SO₄), filtered and concentrated at reduced pressure. The residue was purified by preparative HPLC (Method B) to afford 15 mg (43% yield) of the title compound as a white solid.

¹H NMR (500 MHz, DMSO-d6) δ[ppm] 9.57 (s, 1H), 7.90-7.78 (m, 2H), 7.75-7.64 (m, 1H), 7.46-7.29 (m, 4H), 5.17-5.04 (m, 1H), 1.78-1.59 (m, 2H), 1.58-1.31 (m, 8H), 1.26-1.08 (m, 2H).

LCMS (Analytical Method F): Rt=3.72 mins; MS (ESIpos) m/z=452 (M+H)⁺.

Alternatively, Example 2 can be prepared from cyclopentyl 4-amino-2-(1H-tetrazol-5-yl)benzoate as detailed below.

### Example 2: Cyclopentyl 4-({[1-(3-chlorophenyl)cyclopropyl]carbonyl}amino)-2-(1H-tetrazol-5-yl)benzoate

Cyclopentyl 4-amino-2-(1H-tetrazol-5-yl)benzoate (Int. 17A, 40 mg, 0.15 mmol) was dissolved in dichloromethane (2 mL) and added to 1-(3-chlorophenyl)cyclopropanecarbonyl chloride (37 mg, 0.18 mmol) in pyridine (0.75 mL) at room temperature and stirred for 30 mins. The mixture was diluted with ethyl acetate (30 mL) and washed with 1M aq. HCl (2×20 mL), brine (20 mL), dried (Na₂SO₄), filtered and concentrated at reduced pressure. The residue was purified by preparative HPLC (Method B) to afford 22 mg (33% yield) of the title compound as a white solid.

¹H NMR (500 MHz, DMSO-d6) δ[ppm] 9.67 (s, 1H), 7.97-7.92 (m, 2H), 7.88-7.83 (m, 1H), 7.45-7.41 (m, 1H), 7.40-7.32 (m, 3H), 5.15-5.06 (m, 1H), 1.79-1.64 (m, 2H), 1.57-1.33 (m, 8H), 1.26-1.14 (m, 2H).

LCMS (Analytical Method F): Rt=3.71 mins; MS (ESIpos) m/z=452 (M+H)⁺.

### Example 3: sec-Butyl 4-({[1-(3-chlorophenyl)cyclopropyl]carbonyl}amino)-2-(1H-tetrazol-5-yl)benzoate, as a Mixture of Enantiomers

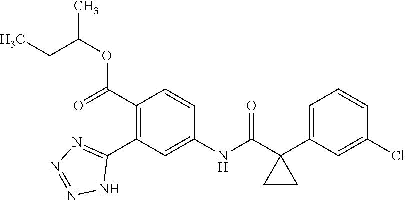 [SMILES: CCC(C)OC(=O)c1ccc(NC(=O)C2(c3cccc(Cl)c3)CC2)cc1-c1nnn[nH]1]

4-({[1-(3-Chlorophenyl)cyclopropyl]carbonyl}amino)-2-(1H-tetrazol-5-yl)benzoic acid (Int. 35A, 30 mg, 0.08 mmol) was dissolved in racemic butan-2-ol (1.5 mL) and concentrated sulfuric acid (1 drop) was added. The mixture was heated at 75° C. and stirring continued for 60 h. The reaction mixture was diluted with EtOAc (10 mL) and washed with brine (3 mL), dried (Na₂SO₄), filtered and concentrated at reduced pressure. The residue was purified by preparative HPLC (Method B) to afford 16 mg (35% yield) of the title compound as a white solid.

¹H NMR (500 MHz, DMSO-d6) δ[ppm] 9.50 (s, 1H), 7.89-7.84 (m, 1H), 7.78-7.72 (m, 1H), 7.55-7.47 (m, 1H), 7.46-7.32 (m, 4H), 4.86-4.69 (m, 1H), 1.52-1.47 (m, 2H), 1.46-1.30 (m, 2H), 1.20-1.14 (m, 2H), 1.06 (d, J=6.3 Hz, 3H), 0.74 (t, J=7.5 Hz, 3H).

LCMS (Analytical Method F): Rt=3.68 mins; MS (ESIpos) m/z=440 (M+H)⁺.

Alternatively, Example 3 can be prepared from sec-butyl 4-amino-2-(1H-tetrazol-5-yl)benzoate as detailed below.

### Example 3: sec-Butyl 4-({[1-(3-chlorophenyl)cyclopropyl]carbonyl}amino)-2-(1H-tetrazol-5-yl)benzoate, as a Mixture of Enantiomers

sec-Butyl 4-amino-2-(1H-tetrazol-5-yl)benzoate (Int. 18A, 40 mg, 0.15 mmol) was dissolved in dichloromethane (2 mL) and added to 1-(3-chlorophenyl)cyclopropanecarbonyl chloride (37 mg, 0.18 mmol) in pyridine (0.75 mL) at room temperature. The mixture was stirred for 30 minutes at RT, before being diluted with ethyl acetate (30 mL) and washed with 1M aq. HCl (2×20 mL), brine (20 mL), dried (Na₂SO₄), filtered and concentrated at reduced pressure. The residue was purified by preparative HPLC (Method B) to afford 30 mg (45% yield) of the title compound as a white solid.

¹H NMR (500 MHz, DMSO-d6) δ[ppm] 9.67 (s, 1H), 7.96 (s, 2H), 7.86 (s, 1H), 7.43 (s, 1H), 7.36 (dd, J=3.8, 2.1 Hz, 3H), 4.83-4.72 (m, 1H), 1.54-1.46 (m, 2H), 1.43-1.32 (m, 2H), 1.24-1.17 (m, 2H), 1.05 (d, J=6.3 Hz, 3H), 0.73 (t, J=7.4 Hz, 3H).

LCMS (Analytical Method F): Rt=3.67 mins; MS (ESIpos) m/z=440 (M+H)⁺.

### Example 4: Cyclopentyl 4-({[1-(3,4-difluorophenyl)cyclopropyl]carbonyl}amino)-2-(1H-tetrazol-5-yl)benzoate

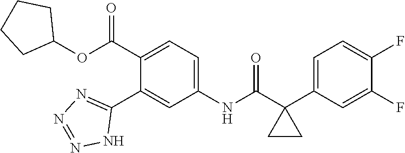 [SMILES: O=C(OC1CCCC1)c1ccc(NC(=O)C2(c3ccc(F)c(F)c3)CC2)cc1-c1nnn[nH]1]

Cyclopentyl 4-amino-2-(1H-tetrazol-5-yl)benzoate (Int. 17A, 40 mg, 0.15 mmol) was dissolved in dichloromethane (2 mL) and added to 1-(3,4-difluorophenyl)cyclopropanecarbonyl chloride (38 mg, 0.18 mmol) in pyridine (0.75 mL) and stirred at RT for 30 mins. The mixture was diluted with ethyl acetate (30 mL) and washed with 1M aq. HCl (2×20 mL), brine (20 mL), dried (Na₂SO₄), filtered and concentrated at reduced pressure. The residue was purified by preparative HPLC (Method B) to afford 19 mg (29% yield) of the title compound as a white solid.

¹H NMR (500 MHz, DMSO-d6) δ[ppm] 9.43 (s, 1H), 8.00-7.91 (m, 2H), 7.87-7.78 (m, 1H), 7.51-7.45 (m, 1H), 7.44-7.36 (m, 1H), 7.29-7.22 (m, 1H), 5.16-5.02 (m, 1H), 1.81-1.66 (m, 2H), 1.55-1.34 (m, 8H), 1.28-1.11 (m, 2H).

LCMS (Analytical Method F): Rt=3.59 mins; MS (ESIPos) m/z=454 (M+H)⁺.

The following compounds were synthesised in an analogous manner to Example 4, starting from the appropriate aniline and acid chloride starting materials.

| Ex. | Structure | Name | Analytics |
|---|---|---|---|
| 5 | 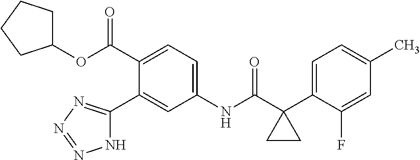 [SMILES: Cc1ccc(C2(C(=O)Nc3ccc(C(=O)OC4CCCC4)c(-c4nnn[nH]4)c3)CC2)c(F)c1] | Cyclopentyl 4- ({[1-(2-fluoro-4- methylphenyl)cyclo- propyl]carbonyl} amino)-2-(1H- tetrazol-5- yl)benzoate | ¹H NMR (500 MHz, DMSO-d6) δ [ppm] 9.24 (s, 1H), 8.01-7.87 (m, 2H), 7.85- 7.75 (m, 1H), 7.39-7.24 (m, 1H), 7.02 (d, J = 9.4 Hz, 2H), 5.14-5.05 (m, 1H), 2.32 (s, 3H), 1.79-1.64 (m, 2H), 1.62- 1.51 (m, 2H), 1.50-1.33 (m, 6H), 1.19- 1.08 (m, 2H). LCMS (Analytical Method F): Rt = 3.67 mins; MS (ESIPos) m/z = 450 (M + H)⁺. |
| 6 | 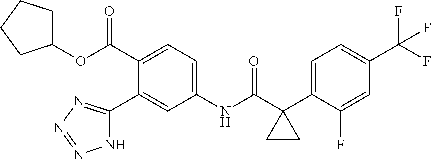 [SMILES: O=C(OC1CCCC1)c1ccc(NC(=O)C2(c3ccc(C(F)(F)F)cc3F)CC2)cc1-c1nnn[nH]1] | Cyclopentyl 4- [({1-[2-fluoro-4- (trifluoromethyl) phenyl]cyclopropyl} carbonyl)amino]- 2-(1H-tetrazol-5- yl)benzoate | ¹H NMR (500 MHz, DMSO-d6) δ [ppm] 9.40 (s, 1H), 8.00-7.90 (m, 2H), 7.84- 7.76 (m, 1H), 7.75-7.63 (m, 2H), 7.63- 7.54 (m, 1H), 5.15-5.06 (m, 1H), 1.77- 1.68 (m, 2H), 1.68-1.64 (m, 2H), 1.49- 1.34 (m, 6H), 1.29-1.24 (m, 2H). LCMS (Analytical Method F): Rt = 3.82 mins; MS (ESIpos) m/z = 504 (M + H)⁺. |
| 7 | 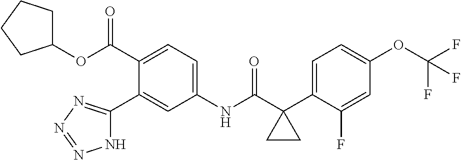 [SMILES: O=C(OC1CCCC1)c1ccc(NC(=O)C2(c3ccc(OC(F)(F)F)cc3F)CC2)cc1-c1nnn[nH]1] | Cyclopentyl 4- [({1-[2-fluoro-4- (trifluoromethoxy) phenyl]cyclopropyl} carbonyl)amino]- 2-(1H-tetrazol-5- yl)benzoate | ¹H NMR (500 MHz, DMSO-d6) δ [ppm] 9.37 (s, 1H), 7.97-7.91 (m, 2H), 7.82- 7.79 (m, 1H), 7.62-7.56 (m, 1H), 7.40- 7.34 (m, 1H), 7.26-7.21 (m, 1H), 5.14- 5.04 (m, 1H), 1.77-1.67 (m, 2H), 1.65- 1.60 (m, 2H), 1.49-1.36 (m, 6H), 1.25- 1.20 (m, 2H). LCMS (Analytical Method F): Rt = 3.91 mins; MS (ESIpos) m/z = 520 (M + H)⁺. |
| 8 | 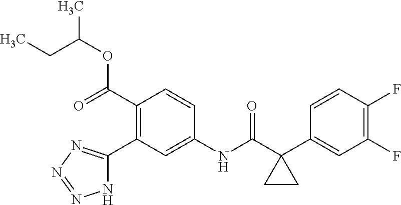 [SMILES: CCC(C)OC(=O)c1ccc(NC(=O)C2(c3ccc(F)c(F)c3)CC2)cc1-c1nnn[nH]1] | sec-Butyl 4-({[1- (3,4- difluorophenyl)cyclo- propyl]carbonyl} amino)-2-(1H- tetrazol-5- yl)benzoate, as a mixture of enantiomers | ¹H NMR (500 MHz, DMSO-d6) δ [ppm] 9.44 (s, 1H), 8.01-7.92 (m, 2H), 7.88- 7.80 (m, 1H), 7.54-7.45 (m, 1H), 7.45- 7.35 (m, 1H), 7.32-7.21 (m, 1H), 4.82- 4.73 (m, 1H), 1.55-1.46 (m, 2H), 1.42- 1.32 (m, 2H), 1.23-1.15 (m, 2H), 1.05 (d, J = 6.3 Hz, 3H), 0.73 (t, J = 7.4 Hz, 3H). LCMS (Analytical Method F): Rt = 3.55 mins; MS (ESIpos) m/z = 442 (M + H)⁺. |
| 9 | 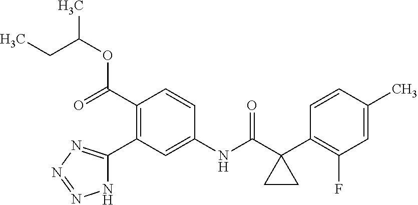 [SMILES: CCC(C)OC(=O)c1ccc(NC(=O)C2(c3ccc(C)cc3F)CC2)cc1-c1nnn[nH]1] | sec-Butyl 4-({[1- (2-fluoro-4- methylphenyl)cyclo- poropyl]carbonyl} amino)-2-(1H- tetrazol-5- yl)benzoate, as a mixture of enantiomers | ¹H NMR (500 MHz, DMSO-d6) δ [ppm] 9.25 (s, 1H), 8.04-7.88 (m, 2H), 7.88- 7.76 (m, 1H), 7.38-7.26 (m, 1H), 7.02 (m, 2H), 4.78 (m, 1H), 2.32 (s, 3H), 1.59- 1.53 (m, 2H), 1.41-1.32 (m, 2H), 1.17- 1.10 (m, 2H), 1.04 (d, J = 6.3 Hz, 3H), 0.73 (t, J = 7.4 Hz, 3H). LCMS (Analytical Method F): Rt = 3.63 mins; MS (ESIpos) m/z = 438 (M + H)⁺. |
| 10 | 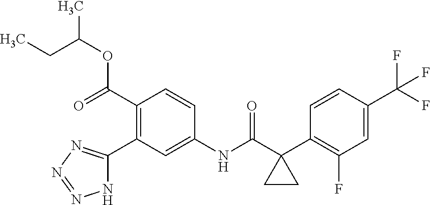 [SMILES: CCC(C)OC(=O)c1ccc(NC(=O)C2(c3ccc(C(F)(F)F)cc3F)CC2)cc1-c1nnn[nH]1] | sec-Butyl 4-[({1- [2-fluoro-4- (trifluoromethyl) phenyl]cyclopropyl} carbonyl)amino]- 2-(1H-tetrazol-5- yl)benzoate, as a mixture of enantiomers | ¹H NMR (500 MHz, DMSO-d6) δ [ppm] 9.41 (s, 1H), 7.98-7.92 (m, 2H), 7.84- 7.78 (m, 1H), 7.73-7.63 (m, 2H), 7.63- 7.58 (m, 1H), 4.82-4.74 (m, 1H), 1.71- 1.63 (m, 2H), 1.42-1.32 (m, 2H), 1.32- 1.24 (m, 2H), 1.05 (d, J = 6.3 Hz, 3H), 0.73 (t, J = 7.4 Hz, 3H). LCMS (Analytical Method F): Rt = 3.79 mins; MS (ESIpos) m/z = 492 (M + H)⁺. |
| 11 | 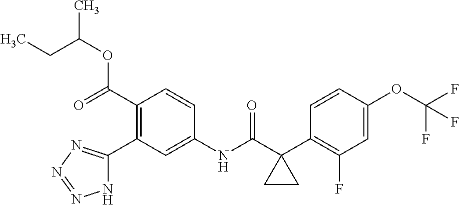 [SMILES: CCC(C)OC(=O)c1ccc(NC(=O)C2(c3ccc(OC(F)(F)F)cc3F)CC2)cc1-c1nnn[nH]1] | sec-Butyl 4-[({1- [2-fluoro-4- (trifluoromethoxy) phenyl]cyclopropyl} carbonyl)amino]- 2-(1H-tetrazol-5- yl)benzoate, as a mixture of enantiomers | ¹H NMR (500 MHz, DMSO-d6) δ [ppm] 9.37 (s, 1H), 8.00-7.93 (m, 2H), 7.84- 7.79 (m, 1H), 7.63-7.55 (m, 1H), 7.42- 7.33 (m, 1H), 7.29-7.21 (m, 1H), 4.83- 4.74 (m, 1H), 1.67-1.59 (m, 2H), 1.42- 1.32 (m, 2H), 1.27-1.20 (m, 2H), 1.05 (d, J = 6.3 Hz, 3H), 0.73 (t, J = 7.5 Hz, 3H). LCMS (Analytical Method F): Rt = 3.86 mins; MS (ESIpos) m/z = 508 (M + H)⁺. |
| 12 | 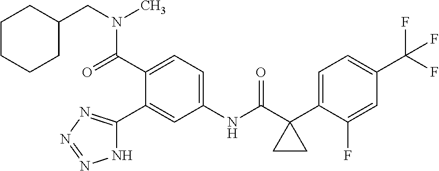 [SMILES: CN(CC1CCCCC1)C(=O)c1ccc(NC(=O)C2(c3ccc(C(F)(F)F)cc3F)CC2)cc1-c1nnn[nH]1] | N- (cyclohexylmethyl)- 4-[({1-[2-fluoro- 4- (trifluoromethyl) phenyl]cyclopropyl} carbonyl)amino]- N-methyl-2-(1H- tetrazol-5- yl)benzamide | ¹H NMR (500 MHz, DMSO-d6) δ 9.30- 9.22 (m, 1H), 8.11-7.98 (m, 1H), 7.80- 7.55 (m, 4H), 7.37-7.27 (m, 1H), 3.21- 2.64 (m, 5H), 1.77-1.37 (m, 8H), 1.33- 0.42 (m, 7H). LCMS (Analytical Method D): Rt = 4.67 mins; MS (ESIPos): m/z = 545 (M + H)⁺. |
| 13 | 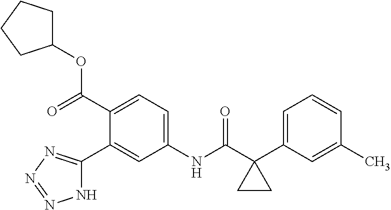 [SMILES: Cc1cccc(C2(C(=O)Nc3ccc(C(=O)OC4CCCC4)c(-c4nnn[nH]4)c3)CC2)c1] | Cyclopentyl-4- ({[2-(3- methylphenyl)cyclo- propyl]carbonyl} amino)-2-(1H- tetrazol-5- yl)benzoate, as a mixture of trans enantiomers | ¹H NMR (500 MHz, DMSO-d6) δ [ppm] 10.77 (s, 1H), 7.97 (d, J = 8.6 Hz, 1H), 7.91 (d, J = 2.0 Hz, 1H), 7.88-7.80 (m, 1H), 7.20-7.14 (m, 1H), 7.04-6.69 (m, 3H), 5.16-5.05 (m, 1H), 2.41-2.32 (m, 1H), 2.27 (s, 3H), 2.16-2.03 (m, 1H), 1.84-1.64 (m, 2H), 1.56-1.35 (m, 8H). LCMS (Analytical Method F): Rt = 3.65 mins; MS (ESIPos) m/z = 432 (M + H)⁺. |

### Example 14: 1-Methylcyclopentyl 4-[({1-[2-fluoro-4-(trifluoromethyl)phenyl]cyclopropyl}-carbonyl)amino]-2-(1H-tetrazol-5-yl)benzoate

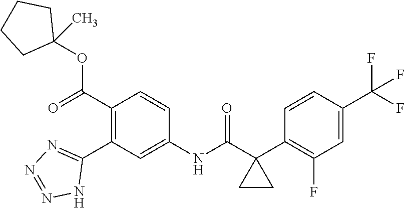 [SMILES: CC1(OC(=O)c2ccc(NC(=O)C3(c4ccc(C(F)(F)F)cc4F)CC3)cc2-c2nnn[nH]2)CCCC1]

4-[({1-[2-fluoro-4-(trifluoromethyl)phenyl]cyclopropyl}carbonyl)amino]-2-(1H-tetrazol-5-yl)benzoic acid (Int. 36A, 30 mg, 0.08 mmol) was dissolved in 1-methylcyclopentanol (1.5 mL) and concentrated sulfuric acid (1 drop) was added. The mixture was heated at 90° C. for 18 h. The reaction mixture was diluted with dichloromethane (10 mL) and washed with brine (2×3 mL). The separated organics were concentrated at reduced pressure and purified by preparative HPLC (Method B) to give 22 mg (62% yield) of the title compound as a white solid.

¹H NMR (500 MHz, DMSO-d6) δ[ppm] 9.33 (s, 1H), 7.88-7.81 (m, 2H), 7.79-7.73 (m, 1H), 7.73-7.63 (m, 2H), 7.63-7.57 (m, 1H), 2.57-2.52 (m, 2H), 2.06-1.94 (m, 2H), 1.86-1.74 (m, 2H), 1.72 (s, 3H), 1.69-1.62 (m, 4H), 1.28-1.22 (m, 2H).

LCMS (Analytical Method F): Rt=4.00 mins; MS (ESIPos) m/z=518 (M+H)⁺.

The following compounds were synthesised in an analogous manner to Example 14, starting from the appropriate carboxylic acid and an alcohol starting materials:

| Ex. | Structure | Name | Analytics |
|---|---|---|---|
| 15 | 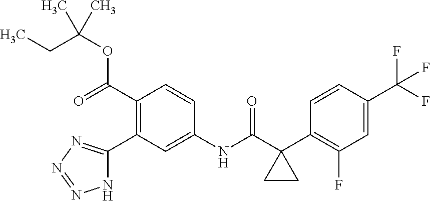 [SMILES: CCC(C)(C)OC(=O)c1ccc(NC(=O)C2(c3ccc(C(F)(F)F)cc3F)CC2)cc1-c1nnn[nH]1] | 2-Methylbutan-2- yl 4-[({1-[2- fluoro-4- (trifluoromethyl) phenyl]cyclopropyl} carbonyl)amino]- 2-(1H-tetrazol-5- yl)benzoate | 1H NMR (500 MHz, DMSO-d6) δ [ppm] 9.33 (s, 1H), 7.88-7.80 (m, 2H), 7.80-7.75 (m, 1H), 7.73-7.64 (m, 2H), 7.62-7.57 (m, 1H), 1.99 (q, J = 7.4 Hz, 2H), 1.69 (s, 6H), 1.68-1.63 (m, 2H), 1.29-1.21 (m, 2H), 0.67 (t, J = 7.4 Hz, 3H). LCMS (Analytical Method F): Rt = 3.91 mins; MS (ESIPos) m/z = 506 (M + H)⁺. |
| 16 | 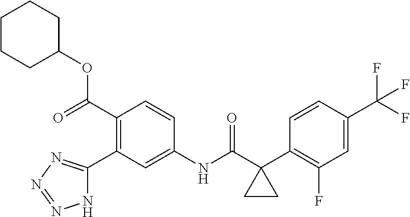 [SMILES: O=C(OC1CCCCC1)c1ccc(NC(=O)C2(c3ccc(C(F)(F)F)cc3F)CC2)cc1-c1nnn[nH]1] | Cyclohexyl 4- [({1-[2-fluoro-4- (trifluoromethyl) phenyl]cyclopropyl} carbonyl)amino]- 2-(1H-tetrazol-5- yl)benzoate | ¹H NMR (500 MHz, DMSO-d6) δ [ppm] 9.27 (s, 1H), 7.83-7.81 (m, 1H), 7.81-7.76 (m, 1H), 7.73-7.54 (m, 4H), 4.77-4.63 (m, 1H), 1.74-1.60 (m, 4H), 1.58-1.38 (m, 3H), 1.33-1.09 (m, 7H). LCMS (Analytical Method F): Rt = 4.03 mins; MS (ESIPos) m/z = 518 (M + H)⁺. |

### Example 17: 2-Methoxyethyl 4-[({1-[2-fluoro-4-(trifluoromethyl)phenyl]cyclopropyl}-carbonyl)amino]-2-(1H-tetrazol-5-yl)benzoate

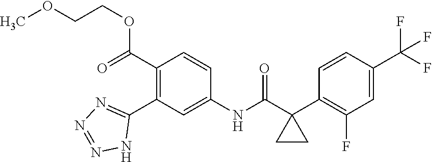 [SMILES: COCCOC(=O)c1ccc(NC(=O)C2(c3ccc(C(F)(F)F)cc3F)CC2)cc1-c1nnn[nH]1]

4-[({1-[2-fluoro-4-(trifluoromethyl)phenyl]cyclopropyl}carbonyl)amino]-2-(1H-tetrazol-5-yl)benzoic acid (Int. 36A, 40 mg, 0.09 mmol) and 2-methoxyethanol (8 mg, 0.11 mmol) were dissolved in DMF (1 mL) and N,N-diisopropylethylamine (48 μL, 0.28 mmol) and HATU (42 mg, 0.11 mmol) were added. The resulting mixture was stirred at room temperature for 18 hours. The mixture was then diluted with dichloromethane (20 mL) and washed with 1M aq. HCl (2×10 mL) and brine (10 mL), dried (Na₂SO₄), filtered and concentrated at reduced pressure. The residue was purified via preparative HPLC (Method A) giving 11 mg (24% yield) of the title compound as a white solid.

¹H NMR (500 MHz, DMSO-d6) δ[ppm] 9.43 (s, 1H), 7.97 (s, 2H), 7.87 (s, 1H), 7.74-7.64 (m, 2H), 7.63-7.58 (m, 1H), 4.25-4.13 (m, 2H), 3.46-3.40 (m, 2H), 3.20 (s, 3H), 1.72-1.62 (m, 2H), 1.32-1.22 (m, 2H).

LCMS (Analytical Method F): Rt=3.36 mins; MS (ESIPos): m/z=494 (M+H)⁺

The following compounds were synthesised in an analogous manner to Example 17, starting from the appropriate carboxylic acid and either an alcohol or an amine starting material.

| Ex. | Structure | Name | Analytical |
|---|---|---|---|
| 18 | 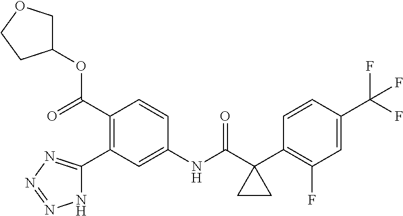 [SMILES: O=C(OC1CCOC1)c1ccc(NC(=O)C2(c3ccc(C(F)(F)F)cc3F)CC2)cc1-c1nnn[nH]1] | Tetrahydrofuran- 3-yl 4-[({1-[2- fluoro-4- (trifluoromethyl) phenyl]cyclo- propyl} carbonyl)amino]- 2-(1H-tetrazol-5- yl)benzoate, as a mixture of enantiomers | ¹H NMR (500 MHz, DMSO-d6) δ [ppm] 9.43 (s, 1H), 8.06-7.89 (m, 2H), 7.89-7.79 (m, 1H), 7.77-7.63 (m, 2H), 7.63-7.50 (m, 1H), 5.31-5.17 (m, 1H), 3.79-3.71 (m, 1H), 3.70-3.62 (m, 1H), 3.59-3.50 (m, 2H), 2.14-1.98 (m, 1H), 1.77-1.63 (m, 3H), 1.32-1.23 (m, 2H). LCMS (Analytical Method F): Rt = 3.32 mins; MS (ESIPos) m/z = 506 (M + H)⁺. |
| 19 | 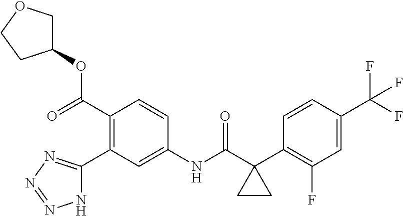 [SMILES: O=C(O[C@H]1CCOC1)c1ccc(NC(=O)C2(c3ccc(C(F)(F)F)cc3F)CC2)cc1-c1nnn[nH]1] | (3S)- Tetrahydrofuran- 3-yl 4-[({1-[2- fluoro-4- (trifluoromethyl) phenyl]cyclo- propyl} carbonyl)amino]- 2-(1H-tetrazol-5- yl)benzoate | ¹H NMR (250 MHz, DMSO-d6) δ [ppm] 1.24-1.32 (m, 2H), 1.68 (m, 2H), 1.74 (m, 1H), 1.98-2.16 (m, 1H), 3.51-3.57 (m, 1H), 3.59 (m, 1H), 3.67 (m, 1H), 3.76 (m, 1H), 5.27 (m, 1H), 7.61 (m, 1H), 7.64-7.76 (m, 2H), 7.86 (m, 1H), 7.98 (m, 2H), 9.43 (s, 1H). LCMS (Analytical Method D): Rt = 4.15 mins; MS (ESIPos): m/z = 506 (M + H)⁺. |
| 20 | 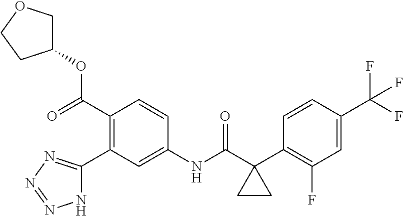 [SMILES: O=C(O[C@@H]1CCOC1)c1ccc(NC(=O)C2(c3ccc(C(F)(F)F)cc3F)CC2)cc1-c1nnn[nH]1] | (3R)- Tetrahydrofuran- 3-yl 4-[({1-[2- fluoro-4- (trifluoromethyl) phenyl]cyclo- propyl} carbonyl)amino]- 2-(1H-tetrazol-5- yl)benzoate | ¹H NMR (500 MHz, DMSO-d6) δ [ppm] 1.24-1.29 (m, 2H), 1.64-1.68 (m, 2H), 1.75 (m, 1H), 2.00-2.10 (m, 1H), 3.49-3.59 (m, 2H), 3.65 (m, 1H), 3.75 (dd, J = 10.4, 4.7 Hz, 1H), 5.27 (m, 1H), 7.60 (d, J = 8.0 Hz, 1H), 7.67 (d, J = 10.6 Hz, 1H), 7.71 (d, J = 7.7 Hz, 1H), 7.84-7.90 (m, 2H), 7.92 (dd, J = 8.7, 2.0 Hz, 1H), 9.39 (s, 1H). LCMS (Analytical Method D): Rt = 4.12 mins; MS (ESIPos): m/z = 506 (M + H)⁺. |
| 21 | 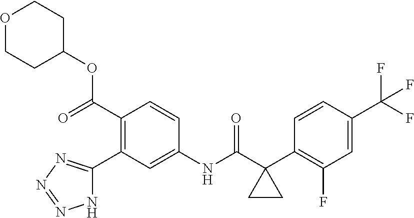 [SMILES: O=C(OC1CCOCC1)c1ccc(NC(=O)C2(c3ccc(C(F)(F)F)cc3F)CC2)cc1-c1nnn[nH]1] | Tetrahydro-2H- pyran-4-yl 4-[({1- [2-fluoro-4- (trifluoromethyl) phenyl]cyclo- propyl} carbonyl)amino]- 2-(1H-tetrazol-5- yl)benzoate | ¹H NMR (500 MHz, DMSO-d6) δ [ppm] 9.42 (s, 1H), 8.05-7.93 (m, 2H), 7.86-7.79 (m, 1H), 7.74-7.64 (m, 2H), 7.62-7.57 (m, 1H), 4.96-4.85 (m, 1H), 3.66-3.58 (m, 2H), 3.43- 3.36 (m,2H), 1.80-1.70 (m, 2H), 1.70- 1.63 (m, 2H), 1.38-1.23 (m, 4H). LCMS (Analytical Method F): Rt = 3.40 mins; MS (ESIPos) m/z = 520 (M + H)⁺. |
| 22 | 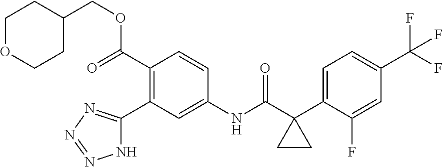 [SMILES: O=C(OCC1CCOCC1)c1ccc(NC(=O)C2(c3ccc(C(F)(F)F)cc3F)CC2)cc1-c1nnn[nH]1] | Tetrahydro-2H- pyran-4-ylmethyl 4-[({1-[2-fluoro- 4- (trifluoromethyl) phenyl]cyclo- propyl} carbonyl)amino]- 2-(1H-tetrazol-5- yl)benzoate | ¹H NMR (500 MHz, DMSO-d6) δ [ppm] 1.11 (m, 2H), 1.25-1.29 (m, 2H), 1.34-1.40 (m, 2H), 1.66 (m, 3H), 3.22 (m, 2H), 3.79 (m, 2H), 3.92 (m, 2H), 7.60 (d, J = 8.1 Hz, 1H), 7.64- 7.73 (m, 2H), 7.85 (m, 1H), 7.95 (m, 2H), 9.41 (s, 1H). LCMS (Analytical Method D): Rt = 4.70 mins; MS (ESIPos): m/z = 534 (M + H)⁺. |
| 23 | 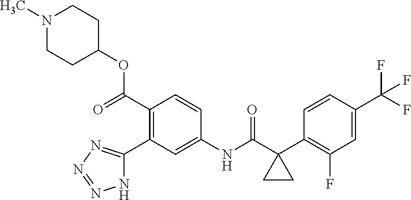 [SMILES: CN1CCC(OC(=O)c2ccc(NC(=O)C3(c4ccc(C(F)(F)F)cc4F)CC3)cc2-c2nnn[nH]2)CC1] | 1- Methylpiperidin- 4-yl 4-[({1-[2- fluoro-4- (trifluoromethyl) phenyl]cyclopropyl} carbonyl)amino]- 2-(1H-tetrazol-5- yl)benzoate | ¹H NMR (500 MHz, DMSO-d6) δ [ppm] 9.28-9.14 (m, 1H), 7.76-7.72 (m, 2H), 7.72-7.64 (m, 2H), 7.63- 7.57 (m, 2H), 4.93-4.84 (m, 1H), 3.02- 2.87 (m, 2H), 2.73-2.62 (m, 5H), 1.90-1.58 (m, 6H), 1.30-1.16 (m, 2H). LCMS (Analytical Method F): Rt = 2.43 mins; MS (ESIPos) m/z = 533 (M + H)⁺. |
| 24 | 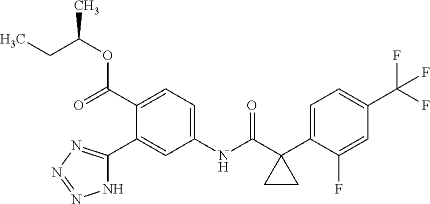 [SMILES: CC[C@@H](C)OC(=O)c1ccc(NC(=O)C2(c3ccc(C(F)(F)F)cc3F)CC2)cc1-c1nnn[nH]1] | (2R)-Butan-2-yl 4- [({1-[2-fluoro-4- (trifluoromethyl) phenyl]cycloproyl} carbonyl)amino]- 2-(1H-tetrazol-5- yl)benzoate | ¹H NMR (500 MHz, DMSO-d6) δ [ppm] 9.37 (s, 1H), 7.97-7.77 (m, 3H), 7.75-7.55 (m, 3H), 4.84-4.72 (m, 1H), 1.68-1.64 (m, 2H), 1.43-1.34 (m, 2H), 1.29-1.24 (m, 2H), 1.05 (d, J = 6.3 Hz, 3H), 0.73 (t, J = 7.5 Hz, 3H). LCMS (Analytical Method F): Rt = 3.79 mins; MS (ESIPos): m/z = 492 (M + H)⁺. |
| 25 | 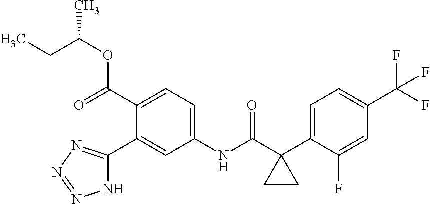 [SMILES: CC[C@H](C)OC(=O)c1ccc(NC(=O)C2(c3ccc(C(F)(F)F)cc3F)CC2)cc1-c1nnn[nH]1] | (2S)-Butan-2-yl 4- [({1-[2-fluoro-4- (trifluoromethyl) phenyl]cyclo- propyl} carbonyl)amino]- 2-(1H-tetrazol-5- yl)benzoate | ¹H NMR (500 MHz, DMSO-d6) δ [ppm] 9.36 (s, 1H), 7.94-7.79 (m, 3H), 7.73-7.64 (m, 2H), 7.62-7.56 (m, 1H), 4.83-4.74 (m, 1H), 1.70-1.63 (m, 2H), 1.42-1.33 (m, 2H), 1.28- 1.23 (m, 2H), 1.05 (d, J = 6.3 Hz, 3H), 0.73 (t, J = 7.5 Hz, 3H). LCMS (Analytical Method F): Rt = 3.80 mins; MS (ESIPos): m/z = 492 (M + H)⁺. |
| 26 | 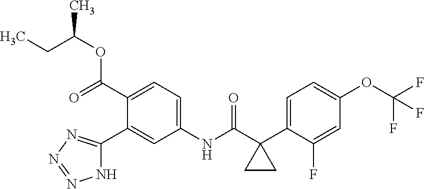 [SMILES: CC[C@@H](C)OC(=O)c1ccc(NC(=O)C2(c3ccc(OC(F)(F)F)cc3F)CC2)cc1-c1nnn[nH]1] | (2R)-Butan-2-yl 4- [({1-[2-fluoro-4- (trifluoromethoxy) phenyl]cyclo- propyl} carbonyl)amino]- 2-(1H-tetrazol-5- yl)benzoate | ¹H NMR (500 MHz, DMSO-d6) δ [ppm] 9.34 (s, 1H), 7.98-7.77 (m, 3H), 7.60 (m, 1H), 7.37 (m, 1H), 7.24 (m, 1H), 4.85-4.71 (m, 1H), 1.67-1.59 (m, 2H), 1.42-1.33 (m, 2H), 1.27- 1.19 (m, 2H), 1.05 (d, J = 6.3 Hz, 3H), 0.73 (t, J = 7.5 Hz, 3H). LCMS (Analytical Method F): Rt = 3.87 mins; MS MS (ESIPos): m/z = 508 (M + H)⁺. |
| 27 | 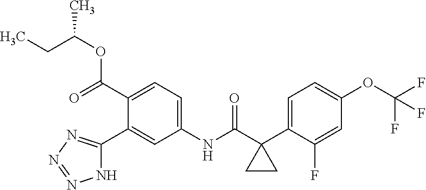 [SMILES: CC[C@H](C)OC(=O)c1ccc(NC(=O)C2(c3ccc(OC(F)(F)F)cc3F)CC2)cc1-c1nnn[nH]1] | (2S)-Butan-2-yl 4- [({1-[2-fluoro-4- (trifluoromethoxy) phenyl]cyclo- propyl} carbonyl)amino]- 2-(1H-tetrazol-5- yl)benzoate | ¹H NMR (500 MHz, DMSO-d6) δ [ppm] 9.35 (s, 1H), 7.97-7.85 (m, 2H), 7.85-7.78 (m, 1H), 7.65-7.55 (m, 1H), 7.43-7.32 (m, 1H), 7.30-7.19 (m, 1H), 4.86-4.72 (m, 1H), 1.66-1.60 (m, 2H), 1.41-1.34 (m, 2H), 1.25-1.19 (m, 2H), 1.05 (d, J = 6.3 Hz, 3H), 0.73 (t, J = 7.5 Hz, 3H). LCMS (Analytical Method F): Rt = |
| | | | 3.87 mins; MS (ESIPos): m/z = 508 |
| | | | (M + H)⁺. |
| 28 | 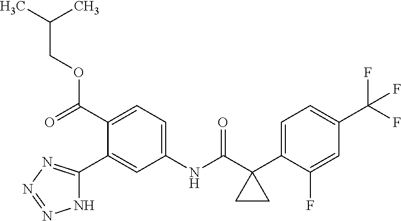 [SMILES: CC(C)COC(=O)c1ccc(NC(=O)C2(c3ccc(C(F)(F)F)cc3F)CC2)cc1-c1nnn[nH]1] | Isobutyl 4-[({1-[2- fluoro-4- (trifluoromethyl) phenyl]cyclo- propyl} carbonyl)amino]- 2-(1H-tetrazol-5- yl)benzoate | ¹H NMR (500 MHz, DMSO-d6) δ [ppm] 9.42 (s, 1H), 7.97 (s, 2H), 7.85 (s, 1H), 7.76-7.63 (m, 2H), 7.63-7.55 (m, 1H), 3.85 (d, J = 6.5 Hz, 2H), 1.76-1.64 (m, 3H), 1.30-1.23 (m, 2H), 0.76 (d, J = 6.7 Hz, 6H). LCMS (Analytical Method F): Rt = 3.85 mins; MS (ESIPos): m/z = 492 (M + H)⁺. |
| 29 | 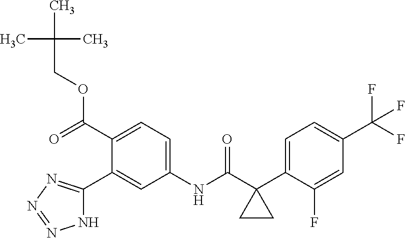 [SMILES: CC(C)(C)COC(=O)c1ccc(NC(=O)C2(c3ccc(C(F)(F)F)cc3F)CC2)cc1-c1nnn[nH]1] | 2,2- Dimethylpropyl 4- [({1-[2-fluoro-4- (trifluoromethyl) phenyl]cyclo- propyl} carbonyl)amino]- 2-(1H-tetrazol-5- yl)benzoate | ¹H NMR (400 MHz, DMSO-d6) δ [ppm] 0.77 (s, 9H), 1.23-1.29 (m, 2H), 1.65-1.68 (m, 2H), 3.78 (s, 2H), 7.61 (dd, 1H), 7.67-7.72 (m, 2H), 7.85 (s, br, 1H), 7.98 (s, br, 2H), 9.44 (s, 1H). LCMS (method 1): Rt = 1.37 min; MS (ESIPos): m/z = 506 (M + H)⁺. |
| 30 | 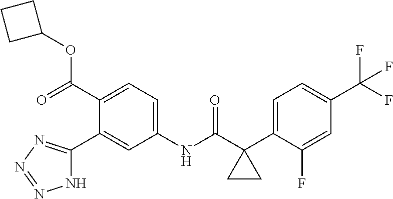 [SMILES: O=C(OC1CCC1)c1ccc(NC(=O)C2(c3ccc(C(F)(F)F)cc3F)CC2)cc1-c1nnn[nH]1] | Cyclobutyl 4-[({1- [2-fluoro-4- (trifluoromethyl) phenyl]cyclo- propyl} carbonyl)amino]- 2-(1H-tetrazol-5- yl)benzoate | ¹H NMR (500 MHz, DMSO-d6) δ [ppm] 9.41 (s, 1H), 7.98-7.93 (m, 2H), 7.85-7.82 (m, 1H), 7.74-7.65 (m, 2H), 7.60 (d, J = 8.0 Hz, 1H), 4.99- 4.88 (m, 1H), 2.25-2.14 (m, 2H), 1.89-1.73 (m, 2H), 1.73-1.62 (m, 3H), 1.62-1.47 (m, 1H), 1.31-1.21 (m, 2H) LCMS (Analytical Method F): Rt = 3.73 mins; MS (ESIPos): m/z = 490 (M + H)⁺. |
| 31 | 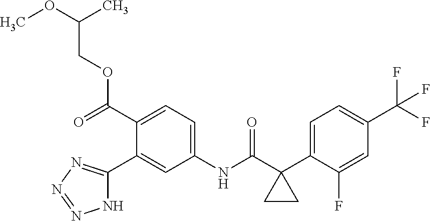 [SMILES: COC(C)COC(=O)c1ccc(NC(=O)C2(c3ccc(C(F)(F)F)cc3F)CC2)cc1-c1nnn[nH]1] | 2-Methoxypropyl 4-[({1-[2-fluoro- 4- (trifluoromethyl) phenyl]cyclo- propyl} carbonyl)amino]- 2-(1H-tetrazol-5- yl)benzoate, as a mixture of enantiomers | ¹H NMR (500 MHz, DMSO-d6) δ [ppm] 9.43 (s, 1H), 8.01-7.95 (m, 2H), 7.89-7.84 (m, 1H), 7.74-7.63 (m, 2H), 7.63-7.57 (m, 1H), 4.07 (m, 1H), 4.00 (m, 1H), 3.40-3.35 (m, 1H), 3.18 (s, 3H), 1.71-1.63 (m, 2H), 1.31-1.24 (m, 2H), 1.01-0.94 (m, 3H). LCMS (Analytical Method F): Rt = 3.49 mins; MS (ESIPos): m/z = 508 (M + H)⁺. |
| 32 | 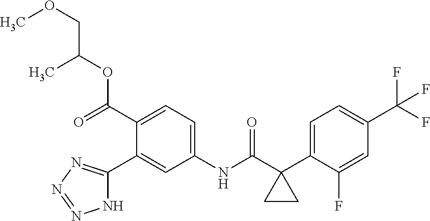 [SMILES: COCC(C)OC(=O)c1ccc(NC(=O)C2(c3ccc(C(F)(F)F)cc3F)CC2)cc1-c1nnn[nH]1] | 1-Methoxypropan- 2-yl 4-[({1-[2- fluoro-4- (trifluoromethyl) phenyl]cyclo- propyl} carbonyl)amino]- 2-(1H-tetrazol-5- yl)benzoate, as a mixture of enantiomers | ¹H NMR (500 MHz, DMSO-d6) δ [ppm] 9.42 (s, 1H), 7.96 (s, 2H), 7.83 (s, 1H), 7.76-7.62 (m, 2H), 7.60 (d, J = 8.1 Hz, 1H), 5.03-4.93 (m, 1H), 3.26- 3.20 (m, 2H), 3.19 (s, 3H), 1.71-1.60 (m, 2H), 1.30-1.22 (m, 2H), 1.05 (d, J = 6.5 Hz, 3H). LCMS (Analytical Method F): Rt = 3.52 mins; MS (ESIPos): m/z = 508 (M + H)⁺. |
| 33 | 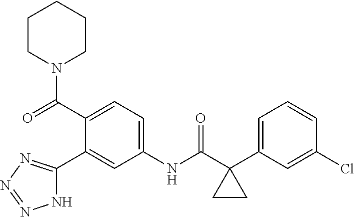 [SMILES: O=C(c1ccc(NC(=O)C2(c3cccc(Cl)c3)CC2)cc1-c1nnn[nH]1)N1CCCCC1] | 1-(3- Chlorophenyl)-N- [4-(piperidin-1- ylcarbonyl)-3- (1H-tetrazol-5- yl)phenyl]cyclo- propane- carboxamide | ¹H NMR (500 MHz, DMSO-d6) δ [ppm] 9.43 (s, 1H), 8.09-7.99 (m, 1H), 7.68 (m, 1H), 7.48-7.40 (m, 1H), 7.40-7.32 (m, 3H), 7.22-7.05 (m, 1H), 3.15-2.77 (m, 4H), 1.60-1.32 (m, 6H), 1.25-1.09 (m, 4H). LCMS (Analytical Method F): Rt = 3.32 mins, MS (ESIPos): m/z = 451 (M + H)⁺. |
| 34 | 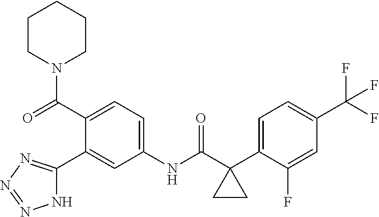 [SMILES: O=C(c1ccc(NC(=O)C2(c3ccc(C(F)(F)F)cc3F)CC2)cc1-c1nnn[nH]1)N1CCCCC1] | 1-[2-Fluoro-4- (trifluoromethyl) phenyl]-N-[4- (piperidin-1- ylcarbonyl)-3- (1H-tetrazol-5- yl)phenyl]cyclo- propane- carboxamide | ¹H NMR (500 MHz, DMSO-d6) δ [ppm] 9.27 (s, 1H), 8.08-8.02 (m, 1H), 7.77-7.73 (m, 1H), 7.73-7.65 (m, 2H), 7.64-7.58 (m, 1H), 7.33 (d, J = 8.4 Hz, 1H), 3.55-3.45 (m ,2H), 3.10- 2.99 (m, 2H), 1.70-1.61 (m, 2H), 1.60-1.42 (m, 4H), 1.34-1.19 (m, 4H). LCMS (Analytical Method F): Rt = 3.49 mins; MS (ESIPos): m/z = 503 (M + H)⁺. |
| 35 | 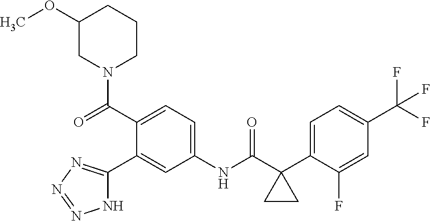 [SMILES: COC1CCCN(C(=O)c2ccc(NC(=O)C3(c4ccc(C(F)(F)F)cc4F)CC3)cc2-c2nnn[nH]2)C1] | 1-[2-Fluoro-4- (trifluoromethyl) phenyl]-N-{4-[(3- methoxypiperidin- 1-yl)carbonyl]-3- (1H-tetrazol-5- yl)phenyl}cyclo- propane- carboxamide, as a mixture of enantiomers | ¹H NMR (500 MHz, DMSO-d6) δ [ppm] 1.18-1.28 (m, 3H), 1.43 (s, 1H), 1.60 (s, 1H), 1.65 (m, 2H), 1.82 (s, 1H), 2.95 (m, 1H), 3.17 (s, 6H), 3.85 (s, 1H), 7.34 (d, J = 8.7 Hz, 1H), 7.61 (d, J = 7.9 Hz, 1H), 7.65-7.78 (m, 3H), 8.06 (m, 1H), 9.27 (s, 1H). LCMS (Analytical Method D): Rt = 4.07 mins; MS (ESIPos): m/z = 533 (M + H)⁺. |
| 36 | 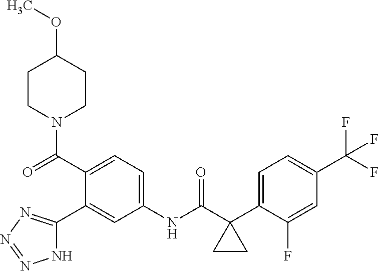 [SMILES: COC1CCN(C(=O)c2ccc(NC(=O)C3(c4ccc(C(F)(F)F)cc4F)CC3)cc2-c2nnn[nH]2)CC1] | 1-[2-Fluoro-4- (trifluoromethyl) phenyl]-N-{4-[(4- methoxypiperidin- 1-yl)carbonyl]-3- (1H-tetrazol-5- yl)phenyl}cyclo- propane- carboxamide | ¹H NMR (500 MHz, DMSO-d6) δ [ppm] 9.29 (s, 1H), 8.14-8.01 (m, 1H), 7.79-7.74 (m, 1H), 7.74-7.67 (m, 2H), 7.64-7.60 (m, 1H), 7.40-7.33 (m, 1H), 3.94-3.83 (m, 1H), 3.23 (s, 6H), 3.02-2.91 (m, 1H), 1.90-1.77 (m, 1H), 1.70-1.56 (m, 3H), 1.51- 1.38 (m, 1H), 1.31-1.17 (m, 3H). LCMS (Analytical Method F): Rt = 3.26 mins; MS (ESIPos): m/z = 533 (M + H)⁺. |
| 37 | 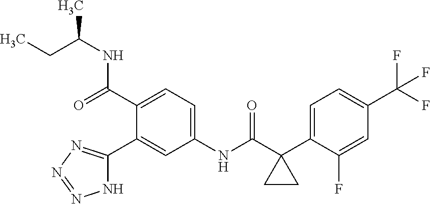 [SMILES: CC[C@@H](C)NC(=O)c1ccc(NC(=O)C2(c3ccc(C(F)(F)F)cc3F)CC2)cc1-c1nnn[nH]1] | N-[(2R)-butan-2- yl]-4-[({1-[2- fluoro-4- (trifluoromethyl) phenyl]cyclo- propyl} carbonyl)amino]- 2-(1H-tetrazol-5- yl)benzamide | ¹H NMR (500 MHz, DMSO-d6) δ [ppm] 9.19 (s, 1H), 8.77 (s, 1H), 7.80-7.55 (m, 6H), 3.78-3.69 (m, 1H), 1.68- 1.60 (m, 2H), 1.47-1.28 (m, 2H), 1.28-1.17 (m, 2H), 1.01 (d, J = 6.6 Hz, 3H), 0.77 (t, J = 7.4 Hz, 3H). LCMS (Analytical Method F): Rt = 3.40 min; MS (ESIPos): m/z = 491 (M + H)⁺. |
| 38 | 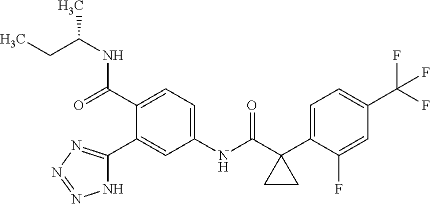 [SMILES: CC[C@H](C)NC(=O)c1ccc(NC(=O)C2(c3ccc(C(F)(F)F)cc3F)CC2)cc1-c1nnn[nH]1] | N-[(2S)-butan-2- yl]-4-[({1-[2- fluoro-4- (trifluoromethyl) phenyl]cyclo- propyl} carbonyl)amino]- 2-(1H-tetrazol-5- y)benzamide | ¹H NMR (500 MHz, DMSO-d6) δ [ppm] 9.27 (s, 1H), 8.09 (d, J = 7.6 Hz, 1H), 7.81 (d, J = 8.4 Hz, 2H), 7.73-7.64 (m, 2H), 7.63-7.52 (m, 2H), 3.77- 3.66 (m, 1H), 1.69-1.60 (m, 2H), 1.49-1.31 (m, 2H), 1.30-1.22 (m, 2H), 1.03 (d, J = 6.6 Hz, 3H), 0.80 (t, J = 7.4 Hz, 3H). LCMS (Analytical Method F): Rt = 3.40 mins; MS (ESIPos): m/z = 491 (M + H)⁺. |
| 39 | 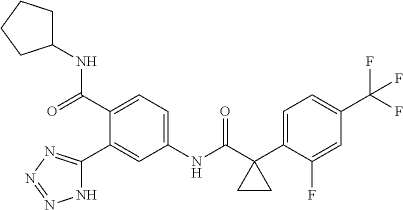 [SMILES: O=C(NC1CCCC1)c1ccc(NC(=O)C2(c3ccc(C(F)(F)F)cc3F)CC2)cc1-c1nnn[nH]1] | N-cyclopentyl-4- [({1-[2-fluoro-4- (trifluoromethyl) phenyl]cyclo- propyl} carbonyl)amino]- 2-(1H-tetrazol-5- yl)benzamide | ¹H NMR (500 MHz, DMSO-d6) δ [ppm] 9.22 (s, 1H), 8.73 (s, 1H), 7.82-7.78 (m, 1H), 7.78-7.73 (m, 1H), 7.73- 7.64 (m, 2H), 7.64-7.55 (m, 2H), 4.13-3.99 (m, 1H), 1.80-1.70 (m, 2H), 1.67-1.62 (m, 2H), 1.62-1.53 (m, 2H), 1.51-1.38 (m, 4H), 1.25- 1.20 (m, 2H). LCMS (Analytical Method F): Rt = 3.47 mins; MS (ESIPos): m/z = 503 (M + H)⁺. |
| 40 | 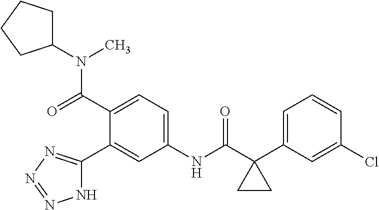 [SMILES: CN(C(=O)c1ccc(NC(=O)C2(c3cccc(Cl)c3)CC2)cc1-c1nnn[nH]1)C1CCCC1] | 4-({[1-(3- Chloro- phenyl)cyclo- propyl]carbonyl} amino)-N- cylcopentyl-N- methyl-2-(1H- tetrazol-5- yl)benzamide | ¹H NMR (500 MHz, DMSO-d6) δ [ppm] 9.52 (s, 1H), 8.18-8.04 (m, 1H), 7.82-7.73 (m, 1H), 7.48-7.42 (m, 1H), 7.41-7.27 (m, 4H), 4.85-3.67 (m, 1H), 2.79-2.52 (m, 3H), 1.78-1.07 (m, 12H). LCMS (Analytical Method F): Rt = 3.54 mins, MS (ESIPos): m/z = 4 65 (M + H)⁺. |
| 41 | 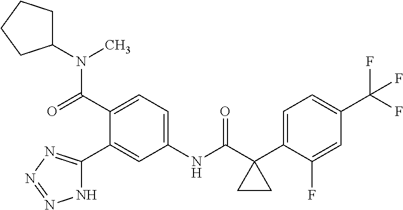 [SMILES: CN(C(=O)c1ccc(NC(=O)C2(c3ccc(C(F)(F)F)cc3F)CC2)cc1-c1nnn[nH]1)C1CCCC1] | N-cyclopentyl-4- [({1-[2-fluoro-4- (trifluoromethyl) phenyl]cyclo- propyl} carbonyl)amino]- N-methyl-2-(1H- tetrazol-5- yl)benzamide | ¹H NMR (500 MHz, DMSO-d6) δ [ppm] 9.22 (s, 1H), 8.07-8.01 (m, 1H), 7.74-7.64 (m, 3H), 7.63-7.57 (m, 1H), 7.27-7.21 (m, 1H), 4.85-3.67 (m, 1H), 2.77 (s, 2H), 1.78-1.59 (m, 4H), 1.59-1.08 (m, 9H). LCMS (Analytical Method F): Rt = 3.67 mins; MS (ESIPos): m/z = 517 (M + H)⁺. |
| 42 | 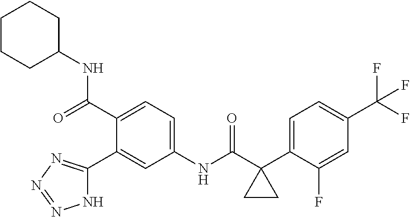 [SMILES: O=C(NC1CCCCC1)c1ccc(NC(=O)C2(c3ccc(C(F)(F)F)cc3F)CC2)cc1-c1nnn[nH]1] | N-cyclohexyl-4- [({1-[2-fluoro-4- (trifluoromethyl) phenyl]cyclo- propyl} carbonyl)amino]- 2-(1H-tetrazol-5- yl)benzamide | ¹H NMR (500 MHz, DMSO-d6) δ [ppm] 9.28 (s, 1H), 8.21-8.08 (m, 1H), 7.88-7.76 (m, 2H), 7.76-7.51 (m, 4H), 3.67-3.49 (m, 1H), 1.81-1.46 (m, 7H), 1.35-0.99 (m, 7H). LCMS (Analytical Method F): Rt = 3.64 mins; MS (ESIPos): m/z = 517 (M + H)⁺. |
| 43 | 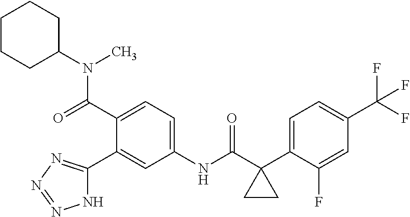 [SMILES: CN(C(=O)c1ccc(NC(=O)C2(c3ccc(C(F)(F)F)cc3F)CC2)cc1-c1nnn[nH]1)C1CCCCC1] | N-cyclohexyl-4- [({1-[2-fluoro-4- (trifluoromethyl) phenyl]cyclo- propyl} carbonyl)amino]- N-methyl-2-(1H- tetrazol-5- yl)benzamide | ¹H NMR (500 MHz, DMSO-d6) δ [ppm] 9.29-9.18 (m, 1H), 8.12-7.94 (m, 1H), 7.80-7.63 (m, 3H), 7.63-7.55 (m, 1H), 7.40-7.22 (m, 1H), 4.26-3.15 (s, 1H), 2.78 (s, 3H), 1.86-1.17 (m, 12H), 1.16-0.77 (m, 2H). LCMS (Analytical Method F): Rt = 3.83 mins; MS (ESIPos): m/z = 531 (M + H)⁺. |
| 44 | 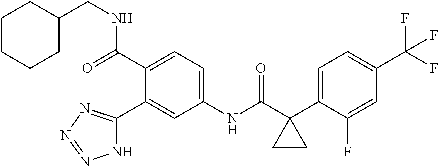 [SMILES: O=C(NCC1CCCCC1)c1ccc(NC(=O)C2(c3ccc(C(F)(F)F)cc3F)CC2)cc1-c1nnn[nH]1] | N-(cyclo- hexylmethyl)- 4-[({1-[2-fluoro- 4- (trifluoromethyl) phenyl]cyclo- propyl} carbonyl)amino]- 2-(1H-tetrazol-5- yl)benzamide | ¹H NMR (500 MHz, DMSO-d6) δ [ppm] 9.27 (s, 1H), 8.43-8.32 (m, 1H), 7.84-7.75 (m, 2H), 7.73-7.64 (m, 2H), 7.63-7.54 (m, 2H), 2.99-2.91 (m, 2H), 1.69-1.55 (m, 7H), 1.48- 1.35 (m, 1H), 1.28-1.21 (m, 2H), 1.21-1.07 (m, 3H), 0.92-0.78 (m, 2H). LCMS (Analytical Method F): Rt = 3.86 mins; MS (ESIPos): m/z = 531 (M + H)⁺. |
| 45 | 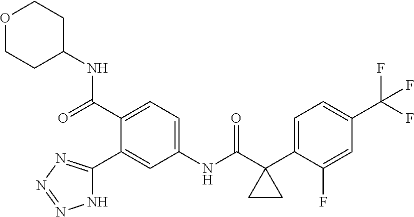 [SMILES: O=C(NC1CCOCC1)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-(tetrahydro-2H- pyran-4-yl)-2- (1H-tetrazol-5- yl)benzamide | ¹H NMR (500 MHz, Chloroform-d) δ [ppm] 8.16 (m, 1H), 7.75 (dm, 1H), 7.71-7.61 (m, 1H), 7.59-7.42 (m, 3H), 7.30 (s, 1H), 6.54 (d, J = 7.7 Hz, 1H), 4.25-4.10 (m, 1H), 4.06-3.97 (m, 2H), 3.62-3.44 (m, 2H), 2.06- 1.91 (m, 2H), 1.89-1.78 (m, 2H), 1.73-1.57 (m, 2H), 1.31-1.20 (m, 2H). LCMS (Analytical Method F): Rt = 3.00 mins; MS (ESIPos): m/z = 519 (M + H)⁺. |
| 46 | 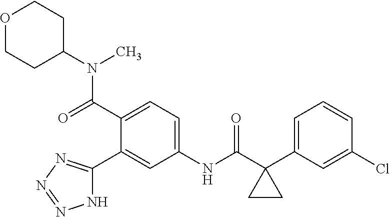 [SMILES: CN(C(=O)c1ccc(NC(=O)C2(c3cccc(Cl)c3)CC2)cc1-c1nnn[nH]1)C1CCOCC1] | 4-({[1-(3- Chloro- phenyl)cyclo- propyl]carbonyl} amino)-N-methyl- N-(tetrahydro-2H- pyran-4-yl)-2- (1H-tetrazol-5- yl)benzamide | ¹H NMR (500 MHz, Methanol-d4) δ [ppm] 8.13-8.07 (m, 1H), 7.74-7.68 (m, 1H), 7.54-7.49 (m, 1H), 7.47-7.33 (m, 4H), 4.73-4.66 (m, 1H), 4.10-3.97 (m, 1H), 3.93-3.77 (m, 1H), 3.58-3.49 (m, 1H), 3.14-3.00 (m, 1H), 2.98-2.64 (m, 3H), 1.94-1.77 (m, 2H), 1.77-1.69 (m, 1H), 1.67-1.60 (m, 2H), 1.58-1.37 (m, 1H), 1.26-1.20 (m, 2H) LCMS (Analytical Method F): Rt = 2.93 mins, MS (ESIPos): m/z = 481 |
| | | | (M + H)⁺. |
| 47 | 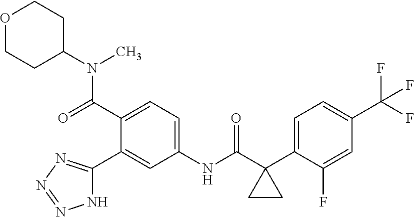 [SMILES: CN(C(=O)c1ccc(NC(=O)C2(c3ccc(C(F)(F)F)cc3F)CC2)cc1-c1nnn[nH]1)C1CCOCC1] | 4-[({1-[2-Fluoro- 4- (trifluoromethyl) phenyl]cyclo- propyl} carbonyl)amino]- N-methyl-N- (tetrahydro-2H- pyran-4-yl)-2- (1H-tetrazol-5- yl)benzamide | ¹H NMR (500 MHz, DMSO-d6) δ [ppm] 9.33-9.22 (m, 1H), 8.15-7.98 (m, 1H), 7.80-7.52 (m, 4H), 7.41-7.27 (m, 1H), 4.61-3.74 (m, 3H), 3.50-3.44 (m, 1H), 3.04-2.90 (m, 1H), 2.84-2.56 (m, 3H), 1.84-1.32 (m, 6H), 1.32-1.16 (m, 2H). LCMS (Analytical Method F): Rt = 3.13 mins; MS (ESIPos): m/z = 533 (M + H)⁺. |
| 48 | 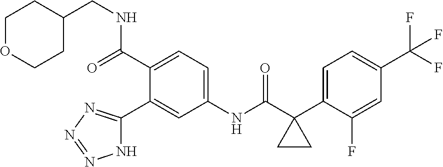 [SMILES: O=C(NCC1CCOCC1)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-(tetrahydro-2H- pyran-4- ylmethyl)-2-(1H- tetrazol-5- yl)benzamide | ¹H NMR (500 MHz, DMSO-d6) δ [ppm] 9.26 (s, 1H), 8.70-8.38 (m, 1H), 7.85-7.74 (m, 2H), 7.74-7.63 (m, 2H), 7.63-7.53 (m, 2H), 3.85-3.74 (m, 2H), 3.27-3.19 (m, 2H), 3.05-2.96 (m, 2H), 1.74-1.60 (m, 3H), 1.55-1.47 (m, 2H), 1.28-1.19 (m, 2H), 1.18-1.04 (m, 2H). LCMS (Analytical Method F): Rt = 3.08 mins; MS (ESIPos): m/z = 533 (M + H)⁺. |
| 49 | 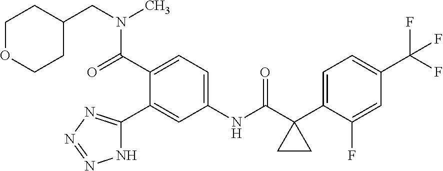 [SMILES: CN(CC1CCOCC1)C(=O)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-methyl-N- (tetrahydro-2H- pyran-4- ylmethyl)-2-(1H- tetrazol-5- | ¹H NMR (500 MHz, Methanol-d₄) δ [ppm] 8.18-8.03 (m, 1H), 7.78-7.63 (m, 2H), 7.62-7.49 (m, 2H), 7.42- 7.32 (m, 1H), 4.02-3.77 (m, 2H), 3.52-3.35 (m, 2H), 3.29-3.25 (m, 1H), 3.09-2.82 (m, 4H), 2.15-1.79 (m, 1H), 1.79-1.73 (m,2H), 1.72-1.66 (m, 1H), 1.53-1.20 (m, 4H), 1.04- 0.89 (m, 1H). LCMS (Analytical Method D): Rt = |
| | | yl)benzamide | 4.05 mins; MS (ESIPos): m/z = 547 |
| | | | (M + H)⁺ |
| 50 | 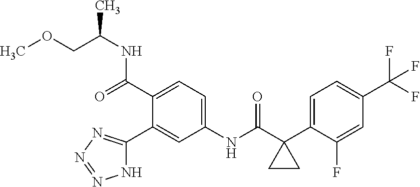 [SMILES: COC[C@@H](C)NC(=O)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-[(2R)-1- methoxypropan-2- yl]-2-(1H-tetrazol- 5-yl)benzamide | ¹H NMR (500 MHz, DMSO-d₆) δ [ppm] 1.04 (d, J = 6.7 Hz, 3H), 1.25 (m, 2H), 1.65 (m, 2H), 3.15 (m, 2H), 3.23 (s, 3H), 4.01 (m, 1H), 7.52-7.63 (m, 2H), 7.64-7.72 (m, 2H), 7.79 (m, 1H), 7.84 (m, 1H), 8.20 (d, J = 7.5 Hz, 1H), 9.27 (s, 1H). LCMS (Analytical Method F): Rt = 4.00 mins; MS (ESIPos): m/z = 507 (M + H)⁺. |
| 51 | 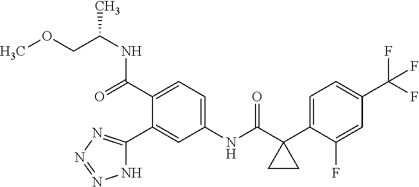 [SMILES: COC[C@H](C)NC(=O)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-[(2S)-1- methoxypropan-2- yl]-2-(1H-tetrazol- 5-yl)benzamide | ¹H NMR (250 MHz, DMSO-d6) δ [ppm] 1.04 (d, J = 6.8 Hz, 3H), 1.24 (m, 2H), 1.65 (m, 2H), 3.10-3.21 (m, 2H), 3.23 (s, 3H), 3.99 (m, 1H), 7.54-7.62 (m, 2H), 7.70 (m, 2H), 7.81 (m, 2H), 9.27 (s, 1H). LCMS (Analytical Method F): Rt = 4.01 mins; MS (ESIPos): m/z = 507 (M + H)⁺. |
| 52 | 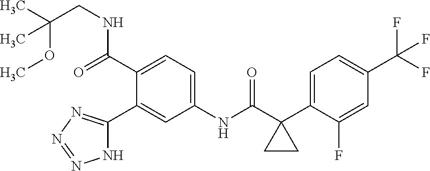 [SMILES: COC(C)(C)CNC(=O)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-(2-methoxy-2- methylpropyl)-2- (1H-tetrazol-5- yl)benzamide | ¹H NMR (500 MHz, DMSO-d6) δ [ppm] 9.28 (s, 1H), 8.29-8.20 (m, 1H), 7.86-7.78 (m, 2H), 7.73-7.64 (m, 2H), 7.63-7.54 (m, 2H), 3.18 (d, J = 6.2 Hz, 2H), 3.09 (s, 3H), 1.71-1.60 (m, 2H), 1.33-1.20 (m, 2H), 1.50 (s, 6H). LCMS (Analytical Method F): Rt = 3.25 mins; MS (ESIPos): m/s = 521 (M + H)⁺. |
| 53 | 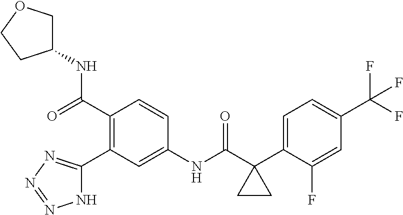 [SMILES: O=C(N[C@@H]1CCOC1)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-[(3R)- tetrahydrofuran-3- yl]-2-(1H-tetrazol- 5-yl)benzamide | ¹H NMR (500 MHz, DMSO-d6) δ [ppm] 1.23-1.27 (m, 2H), 1.63-1.67 (m, 2H), 1.79-1.85 (m, 1H), 2.05 (m, 1H), 3.53 (dd, J = 8.9, 3.9 Hz, 1H), 3.65 (m, 1H), 3.70-3.77 (m, 2H), 4.29 (m, 1H), 7.59 (m, 2H), 7.66-7.73 (m, 2H), 7.80 (dd, J = 8.5, 2.1 Hz, 1H), 7.86 (m, 1H), 8.55 (d, J = 6.2 Hz, 1H), 9.28 (s, 1H). LCMS (Analytical Method F): Rt = 2.94 mins; MS (ESIPos): m/z = 505 (M + H)⁺. |
| 54 | 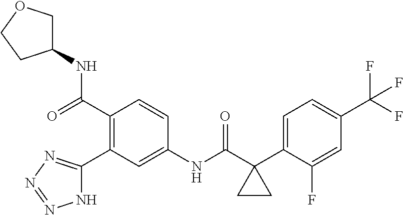 [SMILES: O=C(N[C@H]1CCOC1)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-[(3S)- tetrahydrofuran-3- yl]-2-(1H-tetrazol- 5-yl)benzamide | ¹H NMR (500 MHz, DMSO-d6) δ [ppm] 1.23-1.27 (m, 2H), 1.63-1.68 (m, 2H), 1.78-1.86 (m, 1H), 2.05 (m, 1H), 3.53 (dd, J = 8.9, 3.9 Hz, 1H), 3.65 (m, 1H), 3.70-3.78 (m, 2H), 4.29 (m, 1H), 7.59 (m, 2H), 7.65-7.73 (m, 2H), 7.80 (dd, J = 8.5, 2.0 Hz, 1H), 7.86 (m, 1H), 8.53 (d, J = 6.3 Hz, 1H), 9.28 (s, 1H). LCMS (Analytical Method D): Rt = 4.31 mins; MS (ESIPos): m/z = 505 (M + H)⁺. |
| 55 | 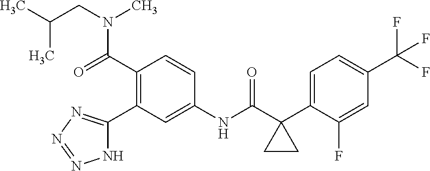 [SMILES: CC(C)CN(C)C(=O)c1ccc(NC(=O)C2(c3ccc(C(F)(F)F)cc3F)CC2)cc1-c1nnn[nH]1] | 4-[({1-[2-Fluoro- 4- (trifluoromethyl) phenyl]cyclo- propyl} carbonyl)amino]- N-isobutyl-N- methyl-2-(1H- tetrazol-5- yl)benzamide | ¹H NMR (500 MHz, DMSO-d6) δ [ppm] 0.77 (m, 6H), 1.24-1.29 (m, 2H), 1.65-1.67 (m, 2H), 1.89 (m, 1H), 2.81 (m, 3H), 3.20 (m, 2H), 7.33 (m, 1H), 7.62 (d, J = 7.9 Hz, 1H), 7.67- 7.80 (m, 3H), 8.05-8.09 (m, 1H), 9.27 (d, J = 7.0 Hz, 1H). LCMS (Analytical Method D): Rt = 4.77 mins; MS (ESIPos): m/z = 505 (M + H)⁺. |
| 56 | 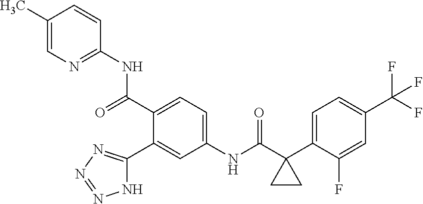 [SMILES: Cc1ccc(NC(=O)c2ccc(NC(=O)C3(c4ccc(C(F)(F)F)cc4F)CC3)cc2-c2nnn[nH]2)nc1] | 4-[({1-[2-Fluoro- 4- (trifluoromethyl) phenyl]cyclo- propyl} carbonyl)amino]- N-(5- methylpyridin-2- yl)-2-(1H-tetrazol- 5-yl)benzamide | ¹H NMR (500 MHz, DMSO-d6) δ [ppm] 10.90 (s, 1H), 9.34 (s, 1H), 8.16 (s, 1H), 8.02-7.87 (m, 2H), 7.87-7.82 (m, 1H), 7.80-7.57 (m, 5H), 2.26 (s, 3H), 1.73-1.62 (m, 2H), 1.32-1.23 (m, 2H). LCMS (Analytical Method F): Rt = 3.16 mins; MS (ESIPos): m/z = 526 (M + H)⁺. |
| 57 | 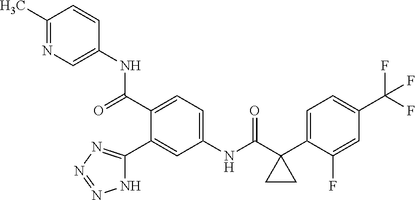 [SMILES: Cc1ccc(NC(=O)c2ccc(NC(=O)C3(c4ccc(C(F)(F)F)cc4F)CC3)cc2-c2nnn[nH]2)cn1] | 4-[({1-[2-Fluoro- 4- (trifluoromethyl) phenyl]cyclo- propyl} carbonyl)amino]- N-(6- methylpyridin-3- yl)-2-(1H-tetrazol- 5-yl)benzamide | ¹H NMR (500 MHz, DMSO-d6) δ [ppm] 10.81 (s, 1H), 9.39 (s, 1H), 8.81 (s, 1H), 8.16-8.08 (m, 1H), 8.04-7.99 (m, 1H), 7.94-7.88 (m, 1H), 7.81-7.66 (m, 3H), 7.64-7.59 (m, 1H), 7.54-7.46 (m, 1H), 1.72-1.65 (m, 2H), 1.32-1.21 (m, 5H). LCMS (Analytical Method F): Rt = 2.46 mins; MS (ESIPos): m/z = 525 (M + H)⁺. |

### Example 58: N-sec-butyl-4-[({1-[2-fluoro-4-(trifluoromethyl)phenyl]cyclopropyl}carbonyl)-amino]-N-methyl-2-(1H-tetrazol-5-yl)benzamide—as single enantiomer 1 (eutomer)

Racemic mixture containing Example 58: N-sec-butyl-4-[({1-[2-fluoro-4-(trifluoromethyl)phenyl]cyclopropyl}-carbonyl)-amino]-N-methyl-2-(1H-tetrazol-5-yl)benzamide, as a mixture of enantiomers

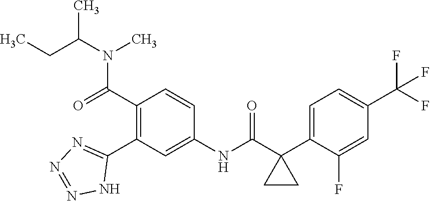 [SMILES: CCC(C)N(C)C(=O)c1ccc(NC(=O)C2(c3ccc(C(F)(F)F)cc3F)CC2)cc1-c1nnn[nH]1]

4-[({1-[2-fluoro-4-(trifluoromethyl)phenyl]cyclopropyl}carbonyl)amino]-2-(1H-tetrazol-5-yl)benzoic acid (80 mg, 0.18 mmol) and racemic N-methylbutan-2-amine (Int. 36A, 20 mg, 0.22 mmol) were dissolved in DMF (2 mL) and N,N-diisopropylethylamine (0.12 mL, 0.64 mmol) and HATU (84 mg, 0.22 mmol) were added. The resulting mixture was stirred at room temperature for 18 hours. The mixture was then diluted with dichloromethane (20 mL) and washed with 1M aq. HCl (2×10 mL) and brine (10 mL), dried (Na₂SO₄), filtered and concentrated at reduced pressure. The residue was purified via preparative HPLC (Method A) giving 69 mg (74% yield) of the title compound as a white solid as a mixture of enantiomers.

¹H NMR (500 MHz, DMSO-d6) δ 9.31-9.11 (m, 1H), 8.06-7.95 (m, 1H), 7.78-7.53 (m, 4H), 7.27-7.13 (m, 1H), 4.59-4.31 (m, 1H), 2.77-2.40 (m, 3H), 1.75-1.17 (m, 6H), 1.13-0.20 (m, 6H).

LCMS (Analytical Method F): Rt=3.58 mins; MS (ESIPos): m/z=505 (M+H)⁺.

### Example 58 (Enantiomer 1 (Eutomer) of N-sec-butyl-4-[({1-[2-fluoro-4-(trifluoromethyl)phenyl]-cyclopropyl}carbonyl)amino]-N-methyl-2-(1H-tetrazol-5-yl)benzamide)

Chiral Purification (Method 1) on 69 mg of the racematic mixture containing Example 58, supra, gave 25 mg of Example 58.

Chiral Analysis (Method 1): >90% e.e. Rt=5.98 mins.

The material obtained was subsequently purified via preparative LC (Method B) giving Example 58 (14 mg).

¹H NMR (500 MHz, DMSO-d6) δ 9.31-9.11 (m, 1H), 8.06-7.95 (m, 1H), 7.78-7.53 (m, 4H), 7.27-7.13 (m, 1H), 4.59-4.31 (m, 1H), 2.77-2.40 (m, 3H), 1.75-1.17 (m, 6H), 1.13-0.20 (m, 6H).

LCMS (Analytical Method F): Rt=3.58 mins; MS (ESIPos): m/z=505 (M+H)+.

### Enantiomer 2 (distomer) of N-sec-butyl-4-[({1-[2-fluoro-4-(trifluoromethyl)phenyl]-cyclopropyl}carbonyl)amino]-N-methyl-2-(1H-tetrazol-5-yl)benzamide2

Chiral Purification (Method 1) on 69 mg of Racemic mixture containing Example 58 gave 26 mg of Distomer (Enantiomer 2).

Chiral Analysis (Method 1): >90% e.e. Rt=6.50 min.

The material obtained was subsequently purified via preparative LC (Method B) giving Enantiomer 2 (9 mg).

¹H NMR (500 MHz, DMSO-d6) δ 9.31-9.11 (m, 1H), 8.06-7.95 (m, 1H), 7.78-7.53 (m, 4H), 7.27-7.13 (m, 1H), 4.59-4.31 (m, 1H), 2.77-2.40 (m, 3H), 1.75-1.17 (m, 6H), 1.13-0.20 (m, 6H).

LCMS (Analytical Method F): Rt=3.57 mins; MS (ESIPos): m/z=505 (M+H)⁺.

### Example 59: Ethyl 2-fluoro-4-[({1-[2-fluoro-4-(trifluoromethyl)phenyl]cyclopropyl}-carbonyl)amino]-6-(1H-tetrazol-5-yl)benzoate

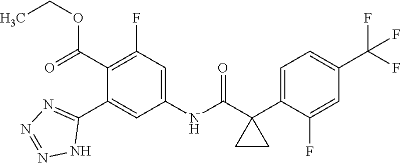 [SMILES: CCOC(=O)c1c(F)cc(NC(=O)C2(c3ccc(C(F)(F)F)cc3F)CC2)cc1-c1nnn[nH]1]

Ethyl 2-cyano-6-fluoro-4-[({1-[2-fluoro-4-(trifluoromethyl)phenyl]cyclopropyl}carbonyl)-amino]benzoate (Int. 33A, 0.79 g, 2.2 mmol) was dissolved in p-xylene (10 mL) and azidotrimethylsilane (0.36 mL, 2.7 mmol) and di-n-butyltin oxide (449 mg, 1.8 mmol) were added. The resulting mixture was heated at 130° C. in a sealed tube for 3 hours giving a yellow solution. The mixture was then cooled to room temperature and methanol (50 mL) was added and the mixture was stirred for 15 minutes. The mixture was then concentrated at reduced pressure and the residue was purified via silica flash column chromatography (eluting with a gradient of eluents; 98:2 to 7:3 DCM/MeOH) giving the title compound (559 mg, 64% yield) as an off white solid.

¹H NMR (500 MHz, DMSO-d6) δ[ppm] 9.50 (s, 1H), 7.95 (d, J=1.4 Hz, 1H), 7.84 (dd, J=12.5, 1.8 Hz, 1H), 7.76-7.64 (m, 2H), 7.61 (d, J=8.1 Hz, 1H), 4.19 (q, J=7.1 Hz, 2H), 1.71-1.62 (m, 2H), 1.36-1.24 (m, 2H), 1.10 (t, J=7.1 Hz, 3H).

LCMS (Analytical Method F): Rt=3.71 mins, MS (ESIPos): m/z=482 (M+H)⁺.

The following compound was synthesised in an analogous manner to Example 59, starting from the appropriate benzonitrile starting material:

| Ex. | Structure | Name | Analytical Data |
|---|---|---|---|
| 60 | 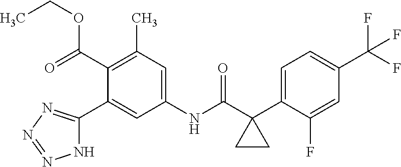 [SMILES: CCOC(=O)c1c(C)cc(NC(=O)C2(c3ccc(C(F)(F)F)cc3F)CC2)cc1-c1nnn[nH]1] | Ethyl 4-[({1-[2- fluoro-4- (trifluoromethyl) phenyl]cyclopropyl} carbonyl)amino]- 2-methyl-6-(1H- tetrazol-5- yl)benzoate | ¹H NMR (500 MHz, DMSO-d6) δ 9.25 (s, 1H), 7.92 (s, 1H), 7.75- 7.52 (m, 4H), 4.12 (q, J = 7.1 Hz, 2H), 1.73-1.57 (m, 2H), 1.32-1.21 (m, 2H), 1.04 (t, J = 7.1 Hz, 3H). LCMS (Analytical Method F): Rt = 3.64 mins, MS (ESIPos): m/z = 478 (M + H)⁺. |

### Example 61: Cyclopentyl 2-fluoro-4-[({1-[2-fluoro-4-(trifluoromethyl)phenyl]cyclopropyl}-carbonyl)amino]-6-(1H-tetrazol-5-yl)benzoate

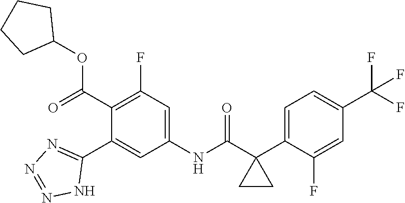 [SMILES: O=C(OC1CCCC1)c1c(F)cc(NC(=O)C2(c3ccc(C(F)(F)F)cc3F)CC2)cc1-c1nnn[nH]1]

2-Fluoro-4-[({1-[2-fluoro-4-(trifluoromethyl)phenyl]cyclopropyl}carbonyl)amino]-6-(1H-tetrazol-5-yl)benzoic acid (Int. 38A, 40 mg, 0.09 mmol) and cyclopentanol (30 mg, 0.35 mmol) were dissolved in DMF (1 mL) and N,N-diisopropylethylamine (46 uL, 0.28 mmol) and HATU (42 mg, 0.11 mmol) were added giving a yellow solution. The mixture was stirred for 3 days at room temperature. The mixture was then diluted with dichloromethane (20 mL) and washed with HCl (1M, 2×10 mL) and brine (10 mL), dried (Na₂SO₄), filtered and concentrated at reduced pressure. The residue was purified via preparative HPLC (Method B) giving the title compound (11 mg, 22% yield) as a white solid. ¹H NMR (500 MHz, DMSO-d6) δ[ppm] 9.36 (s, 1H), 7.88-7.85 (m, 1H), 7.72-7.57 (m, 4H), 5.30-5.20 (m, 1H), 1.82-1.72 (m, 4H), 1.69-1.63 (m, 2H), 1.52-1.43 (m, 4H), 1.26-1.21 (m, 2H).

LCMS (Analytical Method F): Rt=4.06 mins; m/z (ESIPos)=522 (M+H)⁺.

The following compounds were synthesised in an analogous manner to Example 61, starting from the appropriate carboxylic acid and either an alcohol or an amine starting material:

| Ex. | Structure | Name | Analytical Data |
|---|---|---|---|
| 62 | 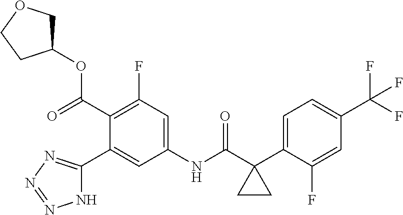 [SMILES: O=C(O[C@H]1CCOC1)c1c(F)cc(NC(=O)C2(c3ccc(C(F)(F)F)cc3F)CC2)cc1-c1nnn[nH]1] | (3S)- tetrahydrofuran-3- yl 2-fluoro-4-[({1- [2-fluoro-4- (trifluoromethyl) phenyl]cyclopropyl} carbonyl)amino]- 6-(1H-tetrazol-5- yl)benzoate | ¹H NMR (500 MHz, DMSO-d6) δ 9.52 (s, 1H), 7.99-7.91 (m, 1H), 7.86 (dd, J = 12.5, 1.8 Hz, 1H), 7.75-7.65 (m, 2H), 7.64- 7.58 (m, 1H), 5.43-5.35 (m, 1H), 3.82-3.73 (m, 1H), 3.71- 3.63 (m, 2H), 3.58- 3.49 (m, 1H), 2.20-2.06 (m, 1H), 1.93-1.81 (m, 1H), 1.73- 1.65 (m, 2H), 1.34-1.24 (m, 2H). LCMS (Analytical Method F): Rt = 3.56 mins, MS (ESIPos): m/z = 524 (M + H)⁺. |
| 63 | 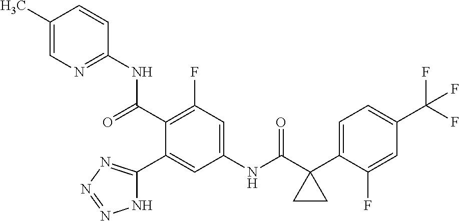 [SMILES: Cc1ccc(NC(=O)c2c(F)cc(NC(=O)C3(c4ccc(C(F)(F)F)cc4F)CC3)cc2-c2nnn[nH]2)nc1] | 2-Fluoro-4-[({1- [2-fluoro-4- (trifluoromethyl) phenyl]cyclopropyl} carbonyl)amino]- N-(5- methylpyridin-2- yl)-6-(1H-tetrazol- 5-yl)benzamide | ¹H NMR (500 MHz, DMSO-d6) δ 11.07 (s, 1H), 9.46 (s, 1H), 8.20- 7.85 (m, 3H), 7.85-7.56 (m, 5H), 2.27 (s, 3H), 1.71-1.63 (m, 2H), 1.34-1.26 (m, 2H). LCMS (Analytical Method F): Rt = 3.41 mins, MS (ESIPos): m/z = 544 (M + H)⁺. |
| 64 | 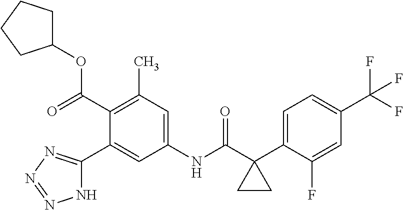 [SMILES: Cc1cc(NC(=O)C2(c3ccc(C(F)(F)F)cc3F)CC2)cc(-c2nnn[nH]2)c1C(=O)OC1CCCC1] | Cyclopentyl 4- [({1-[2-fluoro-4- (trifluoromethyl) phenyl]cyclopropyl} carbonyl)amino]- 2-methyl-6-(1H- tetrazol-5- yl)benzoate | ¹H NMR (500 MHz, DMSO-d6) δ 9.24 (s, 1H), 7.91-7.81 (m, 1H), 7.73-7.56 (m, 4H), 5.22- 5.10 (m, 1H), 2.31 (s, 3H), 1.79- 1.67 (m, 2H), 1.67-1.61 (m, 2H), 1.57-1.34 (m, 6H), 1.28- 1.22 (m, 2H). LCMS (Analytical Method F): Rt = 4.00 mins, MS (ESIPos): m/z = 518 (M + H)⁺. |
| 65 | 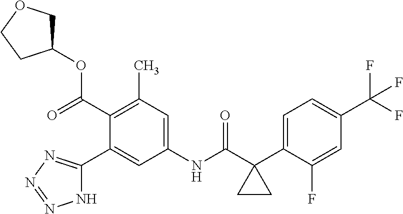 [SMILES: Cc1cc(NC(=O)C2(c3ccc(C(F)(F)F)cc3F)CC2)cc(-c2nnn[nH]2)c1C(=O)O[C@H]1CCOC1] | (3S)- tetrahydrofuran-3- yl 4-[({1-[2- fluoro-4- (trifluoromethyl) phenyl]cyclopropyl} carbonyl)amino]- 2-methyl-6-(1H- tetrazol-5- yl)benzoate | ¹H NMR (500 MHz, DMSO-d6) δ 9.26 (s, 1H), 7.98-7.86 (m, 1H), 7.76-7.54 (m, 4H), 5.39- 5.27 (m, 1H), 3.79-3.70 (m, 1H), 3.70-3.57 (m, 2H), 3.55- 3.46 (m, 1H), 2.32 (s, 3H), 2.13- 2.02 (m, 1H), 1.87-1.77 (m, 1H), 1.70-1.61 (m, 2H), 1.30- 1.22 (m, 2H). LCMS (Analytical Method F): Rt = 3.48 mins, MS (ESIPos): m/z = 520 (M + H)⁺. |
| 66 | 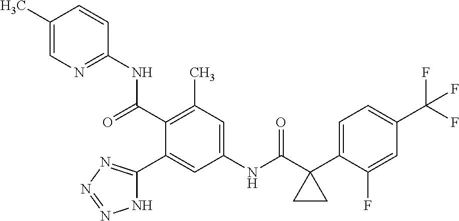 [SMILES: Cc1ccc(NC(=O)c2c(C)cc(NC(=O)C3(c4ccc(C(F)(F)F)cc4F)CC3)cc2-c2nnn[nH]2)nc1] | 4-[({1-[2-fluroo- 4- (trifluoromethyl) phenyl]cyclopropyl} carbonyl)amino]- 2-methyl-N-(5- methylpyridin-2- yl)-6-(1H-tetrazol- 5-yl)benzamide | ¹H NMR (500 MHz, DMSO-d6) δ 10.85 (s, 1H), 9.22 (s, 1H), 8.18- 8.03 (m, 1H), 8.03-7.90 (m, 2H), 7.79-7.64 (m, 3H), 7.64- 7.56 (m, 2H), 2.31 (s, 3H), 2.26 (s, 3H), 1.69-1.62 (m, 2H), 1.28- 1.22 (m, 2H). LCMS (Analytical Method F): Rt = 3.28 mins, MS (ESIPos): m/z = 540 (M + H)⁺. |

### Biological Assays

Compound examples were tested in selected biological assays one or more times. When tested more than once, data are reported as either average values or as median values, wherein
The average value, also referred to as the arithmetic mean value, represents the sum of the values obtained divided by the number of times tested, and
The median value represents the middle number of the group of values when ranked in ascending or descending order. If the number of values in the data set is odd, the median is the middle value. If the number of values in the data set is even, the median is the arithmetic mean of the two middle values.

Compound examples were synthesised one or more times. When synthesised more than once, data from biological assays represent average values or median values calculated utilising data sets obtained from testing of one or more synthetic batch.

The potency to inhibit the Bradykinin B1 receptor was determined for the compound examples of this invention in a cell-based fluorescent calcium-mobilisation assay. The assay measures the ability of compound examples to inhibit Bradykinin B1 receptor agonist-induced increase of intracellular free Ca²⁺ in the cell line expressing Bradykinin B1 receptor. Specifically, calcium indicator-loaded cells are pre-incubated in the absence or presence of different concentrations of compound examples followed by the stimulation with a selective Bradykinin B1 receptor agonist peptide. The change of the intracellular Ca²⁺ concentration is monitored with a fluorescent plate reader FLIPR^{TETRA}®

### (Molecular Devices).

### 1. Calcium Flux Assays

Calcium flux Assays (FLIPR) with cells expressing human Bradykinin B1 receptor (hB1) Calcium flux Assay (FLIPR) with recombinant cells for Bradykinin B1 receptor antagonist, either in the presence (hB1 IC₅₀) or absence (hB1 free IC₅₀) of 0.1% Bovine Serum Albumin (BSA) in assay buffer.

CHO—K1 cell line expressing human B1 receptor was purchased from Euroscreen (Gosselies, Belgium, with reference name hB1-D1). The cells were grown in Nutrient Mixture Ham's F12 (Sigma) containing 10% Foetal bovine serum (Sigma) and 400 μg/mL G418 (Sigma), 5 μg/mL puromycin (Sigma).

Notably, example compounds were tested in the FLIPR assays either in the presence (hB1 IC₅₀) or absence (hB1 free IC₅₀) of 0.1% BSA in assay buffer, in order to assess the potency shifts due to serum protein binding of compounds.

For the calcium flux assay, 80% confluent cells were detached from the culture vessels with Versene (Gibco), and seeded into 384-well plates (Cell binding Surface; Corning, N.Y.; #3683) at a density of 15,000 cells per well. Cells were seeded in a volume of 50 μL in medium without antibiotics and incubated overnight in a humidified atmosphere with 5% CO₂ at 37° C. The following day, the medium was replaced with 20 μL of 5 μM Fluo-4AM dye (Molecular Probes) in assay buffer (2.5 mM probenicid, 1 mg/mL pluronic acid, 135 mM NaCl, 5 mM KCl, 1.8 mM CaCl, 1 mM MgCl₂, 10 mM HEPES, 5.6 mM glucose, and 0.05% gelatine, pH 7.4), which contains or lacks 0.1% BSA for determination of compound potency units as IC₅₀ or free IC₅₀, respectively. The calcium indicator loaded cells were incubated at 37° C. for 2 hrs. Extracellular dye was then removed and each well was filled with 45 μL of assay buffer. Cell plates were kept in dark until used. Example compounds were assayed at 8 concentrations in triplicate. Serial 10-fold dilutions in 100% DMSO were made at a 100-times higher concentration than the final concentration, and then diluted 1:10 in assay buffer. 5 μL of each diluted compound was added to the well of cell plates (yielding final concentration with 1% DMSO), and incubated for 30 min at 28° C. before the addition of B1 receptor agonist on the FLIPR instrument.

Agonist plates contained the agonist Lys-(Des-Arg)-Bradykinin (Bachem, Brackley) at 3.5×EC₉₀ in assay buffer with 1% DMSO. The addition of agonist 20 μl per well to the assay plate was carried out on the FLIPR instrument while continuously monitoring Ca²⁺-dependent fluorescence at 538 nm. A peptide antagonist Lys-(Des-Arg-Leu)-Bradykinin (Bachem, Brackley) at 20 μM was used to determine the full inhibition as control.

Peak fluorescence was used to determine the response to agonist obtained at each concentration of example compound by the following equation:

% Response=100*(RFU_{(example compound)}−RFU_{(control)})/(RFU_{(DMSO)}−RFU_{(control)})
RFU means relative fluorescence units.
Control means full inhibition by the peptide antagonist Lys-(Des-Arg-Leu)-Bradykinin at 20

The response values were plotted against the logarithm of the example compound concentrations. The example compounds were tested in triplicates per plate and mean values were plotted in Excel XLfit to determine IC₅₀ values, percentage of maximal inhibition and the Hill slopes.

IC₅₀ values of example compounds in Calcium flux Assay (FLIPR) as shown in table 1 were determined according to the protocol described above in the absence of 0.1% BSA in assay buffer.

TABLE 1
IC₅₀ values of compound examples in Calcium flux Assay (FLIPR)

| Example No | hB1 free IC₅₀ [nM] |
|---|---|
| 1 | 136 |
| 2 | 21 |
| 3 | 10 |
| 4 | 59 |
| 5 | 25 |
| 6 | 9 |
| 7 | 5 |
| 8 | 21 |
| 9 | 17 |
| 10 | 4 |
| 11 | 3 |
| 12 | 412 |
| 13 | 47 |
| 14 | 999 |
| 15 | 5220 |
| 16 | 5 |
| 17 | 17 |
| 18 | 7 |
| 19 | 13 |
| 20 | 14 |
| 21 | 22 |
| 22 | 18 |
| 23 | 319 |
| 24 | 2 |
| 25 | 3 |
| 26 | 3 |
| 27 | 2 |
| 28 | 3 |
| 29 | 6 |
| 30 | 2 |
| 31 | 9 |
| 32 | 13 |
| 33 | 633 |
| 34 | 70 |
| 35 | 341 |
| 36 | 523 |
| 37 | 121 |
| 38 | 80 |
| 39 | 34 |
| 40 | 1240 |
| 41 | 205 |
| 42 | 34 |
| 43 | 50 |
| 44 | 13 |
| 45 | 133 |
| 46 | 3300 |
| 47 | 445 |
| 48 | 60 |
| 49 | 1800 |
| 50 | 89 |
| 51 | 409 |
| 52 | 143 |
| 53 | 3610 |
| 54 | 1530 |
| 55 | 1770 |
| 56 | 50 |
| 57 | 29 |
| 58 | 1850 |
| 59 | 5 |
| 60 | 26 |
| 61 | 2 |
| 62 | 2 |
| 63 | 51 |
| 64 | 25 |
| 65 | 62 |
| 66 | 1450 |

1. A compound of general formula (I): 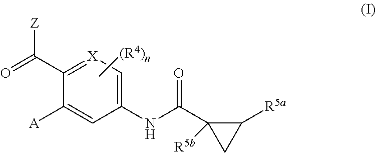
in which
A represents tetrazolyl which is attached to the rest of the molecule via the carbon atom;
X represents CH, CR⁶ or N;
Z represents OR¹ or NR²R³;
R¹ represents C₁-C₇-alkyl, C₃-C₇-cycloalkyl, 4- to 7-membered heterocycloalkyl, (4- to 7-membered heterocycloalkyl)-C₁-C₄-alkyl, phenyl or 5- or 6-membered heteroaryl, wherein said C₁-C₇-alkyl is optionally substituted, one or more times, independently from each other, with C₁-C₄-alkoxy and said C₃-C₇-cycloalkyl, 4- to 7-membered heterocycloalkyl, (4- to 7-membered heterocycloalkyl)-C₁-C₄-alkyl, phenyl or 5- or 6-membered heteroaryl groups are optionally substituted, one or more times, independently from each other, with C₁-C₄-alkyl or C₁-C₄-alkoxy;
R², R³ represent, independently from each other, hydrogen, C₁-C₇-alkyl, C₃-C₇-cycloalkyl, 4- to 7-membered heterocycloalkyl, C₃-C₇-cycloalkyl-C₁-C₄-alkyl, (4- to 7-membered heterocycloalkyl)-C₁-C₄-alkyl, phenyl or 5- or 6-membered heteroaryl,
wherein said C₁-C₇-alkyl is optionally substituted, one or more times, independently from each other, with C₁-C₄-alkoxy and said C₃-C₇-cycloalkyl, 4- to 7-membered heterocycloalkyl, C₃-C₇-cycloalkyl-C₁-C₄-alkyl, (4- to 7-membered heterocycloalkyl)-C₁-C₄-alkyl, phenyl or 5- or 6-membered heteroaryl groups are optionally substituted, one or more times, independently from each other, with C₁-C₄-alkyl or C₁-C₄-alkoxy, or
R² and R³ together with the nitrogen atom to which they are attached form a 5- to 6-membered heterocyclic ring, optionally containing one additional heteroatom selected from O and N and optionally substituted with C₁-C₄-alkyl or C₁-C₄-alkoxy;
R⁴ represents C₁-C₄-alkyl or halogen;
R^{5a} represents hydrogen, phenyl or heteroaryl;
wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy;
R^{5b} represents hydrogen, phenyl or heteroaryl;
wherein said phenyl or heteroaryl groups are optionally substituted, one or more times, independently from each other, with halogen, C₁-C₄-alkyl, C₁-C₄-alkoxy, C₁-C₄-haloalkyl or C₁-C₄-haloalkoxy and
wherein one of R^{5a} and R^{5b} represents hydrogen and the other represents phenyl or heteroaryl;
R⁶ represents C₁-C₃-alkyl or halogen;
n represents 0, 1 or 2;
and stereoisomers, tautomers, N-oxides, hydrates, solvates, and salts thereof, and mixtures of same.

2.-17. (canceled)
